(19) United States
(12) Patent Application Publication
(10) Pub. No.: US 2024/0277715 A1
(43) Pub. Date: Aug. 22, 2024

(54) COMBINATION THERAPIES COMPRISING A METTL3 INHIBITOR AND A FURTHER ANTICANCER AGENT

(71) Applicant: Storm Therapeutics Limited, Cambridge (GB)

(72) Inventors: Oliver Lars Rausch, Cambridge (GB); Mark Robert Albertella, Cambridge (GB); Yaara Ofir-Rosenfeld, Cambridge (GB); Lina Vasiliauskaite, Cambridge (GB)

(21) Appl. No.: 18/565,336

(22) PCT Filed: Jun. 1, 2022
(86) PCT No.: PCT/GB2022/051400

§ 371 Date: Nov. 29, 2023

(30) Foreign Application Priority Data
Jun. 2, 2021 (GB) 2107907.4

(51) Int. Cl. A61K 31/519; A61K 31/44; A61K 31/4545; A61K 31/4709; A61K 31/497; A61K 31/553; A61K 31/635; A61K 31/704; A61K 31/706; A61K 31/7068; A61K 35/76; A61K 39/00; A61K 39/395; A61K 45/06; A61P 35/00; A61P 35/02

(52) U.S. Cl. CPC A61K 31/519; A61K 31/44; A61K 31/4545; A61K 31/4709; A61K 31/497; A61K 31/553; A61K 31/635; A61K 31/704; A61K 31/706; A61K 31/7068; A61K 35/76; A61K 39/0011; A61K 39/3955; A61K 39/4611; A61K 39/4631; A61K 39/4644; A61K 45/06; A61P 35/00; A61P 35/02

(57) ABSTRACT

The present invention relates to novel combination therapies for the treatment of proliferative disorders, such as, for example, cancer. The combination therapies comprises the administration of a METTL3 inhibitor in combination with
(i) an immune oncology agent or therapy;
(ii) a BCL2 inhibitor, or a pharmaceutically acceptable salt thereof;
(iii) an anthracycline topoisomerase 2 inhibitor, or a pharmaceutically acceptable salt thereof;
(iv) cytarabine, or a pharmaceutically acceptable salt thereof;
(v) a hypomethylating agent, or a pharmaceutically acceptable salt thereof; or
(vi) a FLT3 inhibitor, or a pharmaceutically acceptable salt thereof.

### FIELD OF THE INVENTION

The present invention relates to novel combination therapies for the treatment of cancer.

### BACKGROUND OF THE INVENTION

Cancer is caused by uncontrolled and unregulated cellular proliferation. Precisely what causes a cell to become malignant and proliferate in an uncontrolled and unregulated manner has been the focus of intense research over recent decades. This research has led to the identification of a number of molecular targets and key metabolic pathways that are known to be associated with malignancy.

Despite numerous advances in the treatment of cancer, there remains a need for new therapies that provide improved therapeutic outcomes.

One particular target that is attracting interest is METTL3. N⁶-methyladenosine (m⁶A) is an abundant internal RNA modification that is catalysed predominantly by the METTL3-METTL14 methyltransferase complex. The m⁶A methyltransferase METTL3 has been linked to the initiation and maintenance of acute myeloid leukaemia (AML). Yankova et al. (Nature: volume 593, pages 597-601 (2021)) describe the identification and characterisation of STM2457, a highly potent and selective first-in-class catalytic inhibitor of METTL3. Treatment with STM2457 resulted in a reduction in AML growth and an increase in differentiation and apoptosis. These cellular effects are accompanied by selective reduction of m⁶A levels on known leukaemogenic mRNAs and a decrease in their expression consistent with a translational defect. The pharmacological inhibition of METTL3 in vivo led to impaired engraftment and prolonged survival in various mouse models of AML, specifically targeting key stem cell subpopulations of AML. METTL3 is therefore a potential therapeutic strategy against AML, and the targeting of RNA-modifying enzymes represents a promising avenue for anticancer therapy more generally.

International Patent Publication No. WO2020/050898 describes further small molecule METTL3 inhibitors that are suitable for the treatment of cancer.

METTL3 inhibitors clearly show promise for the treatment of AML and other cancers. However, there is always a need to identify new therapeutic strategies that can be used to more improve therapeutic outcomes.

The present invention was devised with the foregoing in mind.

### SUMMARY OF THE INVENTION

1. Combinations of METTL3 Inhibitors with Immuno-Oncology Agents (e.g. Immune Checkpoint Inhibitors)

Data presented in the example section herein shows that the administration of the METTL3 inhibitor compound STM3480 in combination with an immune checkpoint inhibitor (anti-PD1 and anti-PD-L1) resulted in a significant increase in the observed anti-tumour immune response (when compared to vehicle controls and the treatment with either the METTL3 inhibitor or the immune checkpoint inhibitor alone). Data is presented for tumour killing co-culture cell assays (see Example 1) as well as in vivo studies using the A20 B-cell lymphoma model, EMT6 breast cancer model and CT26 colorectal cancer model (see Example 2). Collectively, these data suggest that the administration of a METTL3 inhibitor together with an immune checkpoint inhibitor significantly enhances the anti-tumour immune response. METTL3 inhibition may therefore play a role in sensitising the cancer cells to immune checkpoint inhibitor therapy.

Thus, the present invention relates, in one aspect, to a METTL3 inhibitor as defined herein, or a pharmaceutically acceptable salt thereof, for use as an immune-sensitiser.

The present invention also relates to the use of a METTL3 inhibitor as defined herein, or a pharmaceutically acceptable salt thereof, in the manufacture of a medicament for use as an immune-sensitiser.

The present invention also relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is administered in combination with an immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BITE), oncolytic viruses, cancer vaccines, and/or CAR-T cell therapy).

The present invention also relates to the use of a METTL3 inhibitor as defined herein, or a pharmaceutically acceptable salt thereof, in the manufacture of a medicament for use in the treatment of cancer, wherein the METTL3 inhibitor as defined herein, or a pharmaceutically acceptable salt thereof, is administered in combination with an immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BiTE), oncolytic viruses, cancer vaccines, and/or CAR-T cell therapy).

The present invention also relates to a method of treating cancer, the method comprising administering a therapeutically effective amount of a METTL3 inhibitor as defined herein, or a pharmaceutically acceptable salt thereof, to a patient in combination with an immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BiTE), oncolytic viruses, cancer vaccines, and/or CAR-T cell therapy).

Suitably, the METTL3 inhibitor as defined herein, or a pharmaceutically acceptable salt thereof, is administered simultaneously, sequentially or separately with the immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BiTE), oncolytic viruses, cancer vaccines, and/or CAR-T cell therapy.

In one aspect the present invention relates to a combination comprising a METTL3 inhibitor as defined herein, or a pharmaceutically acceptable salt thereof, and an immune checkpoint inhibitor, or a pharmaceutically acceptable salt thereof.

In another aspect the present invention relates to a pharmaceutical product comprising a combination as defined herein.

In another aspect, the present invention relates to a pharmaceutical composition comprising a combination as defined herein, and one or more pharmaceutically acceptable excipients.

In another aspect, the present invention relates to a combination as defined herein, or a pharmaceutical product as defined herein, or a pharmaceutical composition as defined herein for use in therapy.

In another aspect, the present invention relates to a combination as defined herein, or a pharmaceutical product as defined herein, or a pharmaceutical composition as defined herein for use in the treatment of cancer.

In another aspect, the present invention relates to a use of a combination as defined herein in the manufacture of a medicament for the treatment of cancer.

In another aspect, the present invention relates to a method of treating of cancer in a subject in need thereof comprising administering to said subject a therapeutically effective amount of a combination as defined herein.

In another aspect, the present invention relates to a method of potentiating the immune response to a tumour, the method comprising administering to a patient in need of such treatment a therapeutically effective amount of a combination as defined herein.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with an immune checkpoint inhibitor, or a pharmaceutically acceptable salt thereof.

In another aspect, the present invention relates to an immune checkpoint inhibitor, or a pharmaceutically acceptable salt thereof, for use in the treatment of cancer, wherein the immune checkpoint inhibitor is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with an immune checkpoint inhibitor, or a pharmaceutically acceptable salt thereof.

In another aspect, the present invention relates to a use of an immune checkpoint inhibitor, or a pharmaceutically acceptable salt thereof in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and an immune checkpoint inhibitor, or a pharmaceutically acceptable salt thereof, wherein the METTL3 inhibitor as defined herein, or a pharmaceutically acceptable salt thereof, and the immune checkpoint inhibitor, or a pharmaceutically acceptable salt thereof are administered sequentially, separately or simultaneously with one another.

In another aspect, the present invention relates to a method of treating cancer or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with an immune checkpoint inhibitor, or a pharmaceutically acceptable salt thereof.

In another aspect, the present invention relates to a method of potentiating the immune response to a tumour, the method comprising administering to a patient in need of such treatment a therapeutically effective amount of a METTL3 inhibitor as defined herein, or a pharmaceutically acceptable salt thereof, and an immune checkpoint inhibitor, or a pharmaceutically acceptable salt thereof, wherein the METTL3 inhibitor as defined herein, or a pharmaceutically acceptable salt thereof, and the immune checkpoint inhibitor, or a pharmaceutically acceptable salt thereof are administered sequentially, separately or simultaneously with one another.

Suitably, the cancer is a solid tumour.

2. Combinations of METTL3 Inhibitors with BCL2 Inhibitors (e.g. Venetoclax)

Data presented in the example section shows that the METTL3 inhibitor compounds STM3480, STM3006 and STM3675, when administered in combination with a BCL2 inhibitor (venetoclax), produced a synergistic increase in the observed anticancer effect. Data is presented for Kasumi1 and MOLM13 AML cell lines (see Example 3). Collectively, these data suggest that METTL3 inhibition synergistically enhances the antitumour effect of BCL2 inhibitor (e.g. venetoclax) therapy. The combination of a METTL3 inhibitor with a BCL2 inhibitor (e.g. venetoclax) therefore offers a promising therapy for diseases or conditions in which BCL2 inhibitor therapy is beneficial (e.g. the treatment of cancers such as acute myeloid leukaemia (AML), chronic lymphocytic leukemia (CLL), small lymphocytic lymphoma (SLL) and myelodysplastic syndromes (MDS)).

Thus, the present invention also relates to a METTL3 inhibitor as defined herein, or a pharmaceutically acceptable salt thereof, for use in the treatment of diseases or conditions in which BCL2 inhibitor therapy is beneficial (e.g. the treatment of cancer), wherein the METTL3 inhibitor as defined herein, or a pharmaceutically acceptable salt thereof, is administered in combination with a BCL2 inhibitor (e.g. venetoclax).

The present invention also relates to the use of a METTL3 inhibitor as defined herein, or a pharmaceutically acceptable salt thereof, in the manufacture of a medicament for use in the treatment of diseases or conditions in which BCL2 inhibitor therapy is beneficial (e.g. the treatment of cancer), wherein the METTL3 inhibitor as defined herein, or a pharmaceutically acceptable salt thereof, is administered in combination with a BCL2 inhibitor (e.g. venetoclax).

The present invention also relates to a method of treating diseases or conditions in which BCL2 inhibitor therapy is beneficial (e.g. the treatment of cancer), the method comprising administering a therapeutically effective amount of a METTL3 inhibitor as defined herein, or a pharmaceutically acceptable salt thereof, to a patient in combination with a BCL2 inhibitor (e.g. venetoclax).

Suitably, the METTL3 inhibitor as defined herein, or a pharmaceutically acceptable salt thereof, is administered simultaneously, sequentially or separately with the BCL2 inhibitor (e.g. venetoclax) therapy.

In one aspect the present invention relates to a combination comprising a METTL3 inhibitor as defined herein, or a pharmaceutically acceptable salt thereof, and a BCL2 inhibitor (e.g. venetoclax), or a pharmaceutically acceptable salt thereof.

In another aspect the present invention relates to a pharmaceutical product comprising a combination as defined above.

In another aspect, the present invention relates to a pharmaceutical composition comprising a combination as defined above, and one or more pharmaceutically acceptable excipients.

In another aspect, the present invention relates to a combination as defined above, or a pharmaceutical product as defined above, or a pharmaceutical composition as defined above for use in therapy.

In another aspect, the present invention relates to a combination as defined herein, or a pharmaceutical product as defined above, or a pharmaceutical composition as defined above for use in the treatment of diseases or conditions in which BCL2 inhibitor therapy is beneficial (e.g. the treatment of cancer).

In another aspect, the present invention relates to the use of a combination as defined above in the manufacture of a medicament for the treatment of diseases or conditions in which BCL2 inhibitor therapy is beneficial (e.g. the treatment of cancer).

In another aspect, the present invention relates to a method of treating of diseases or conditions in which BCL2 inhibitor therapy is beneficial (e.g. the treatment of cancer) in a subject in need thereof comprising administering to said subject a therapeutically effective amount of a combination as defined above.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with a BCL2 inhibitor (e.g. venetoclax), or a pharmaceutically acceptable salt thereof.

In another aspect, the present invention relates to a BCL2 inhibitor (e.g. venetoclax), or a pharmaceutically acceptable salt thereof, for use in the treatment of cancer, wherein the BCL2 inhibitor (e.g. venetoclax), or a pharmaceutically acceptable salt thereof, is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with a BCL2 inhibitor (e.g. venetoclax), or a pharmaceutically acceptable salt thereof.

In another aspect, the present invention relates to a use of a BCL2 inhibitor (e.g. venetoclax), or a pharmaceutically acceptable salt thereof, in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and a BCL2 inhibitor (e.g. venetoclax), or a pharmaceutically acceptable salt thereof, wherein the METTL3 inhibitor as defined herein, or a pharmaceutically acceptable salt thereof, and the BCL2 inhibitor (e.g. venetoclax), or a pharmaceutically acceptable salt thereof, are administered sequentially, separately or simultaneously with one another.

In another aspect, the present invention relates to a method of treating cancer, or potentiating the effect of a BCL2 inhibitor (e.g. venetoclax), or a pharmaceutically acceptable salt thereof, the method comprising administering to a patient in need of such treatment a therapeutically effective amount the BCL2 inhibitor (e.g. venetoclax), or a pharmaceutically acceptable salt thereof, separately, sequentially or simultaneously with a METTL3 inhibitor as defined herein, or a pharmaceutically acceptable salt thereof.

In another aspect, the present invention relates to a method of treating cancer or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with a BCL2 inhibitor (e.g. venetoclax), or a pharmaceutically acceptable salt thereof.

Suitably, the cancer is selected from acute myeloid leukaemia (AML), chronic lymphocytic leukaemia (CLL), small lymphocytic lymphoma (SLL) and myelodysplastic syndromes (MDS).

3. Combinations of METTL3 Inhibitors with Anthracycline Topoisomerase 2 Inhibitors (e.g. Daunorubicin), Cytarabine, Hypomethylating Agents (e.g. 5-Azacitidine or Decitabine) or FLT3 Inhibitors (e.g. Quizartinib)

Data presented in the example section herein shows that the METTL3 inhibitor compounds STM3480 and STM3006, when administered in combination with various standard of care drugs for the treatment of AML, such as daunorubicin, cytarabine, 5-azacitidine and quizartinib, produced an enhanced therapeutic effect in Kasumi1 or MOLM-14 AML cell lines (see Example 4). Collectively, these data suggest that the administration of a METTL3 inhibitor enhances the antitumour effects of AML standard of care agents, including anthracycline topoisomerase 2 inhibitors (e.g. daunorubicin), cytarabine, hypomethylating agents (e.g. 5-azacitidine or decitabine), and/or FLT3 inhibitors (e.g. quizartinib).

The combination of a METTL3 inhibitor with either an anthracycline topoisomerase 2 inhibitor (e.g. daunorubicin), cytarabine, a hypomethylating agent (e.g. 5-azacitidine or decitabine), and/or a FLT3 inhibitors (e.g. quizartinib) therefore offers a promising therapeutic strategies for the treatment of cancer (e.g. acute myeloid leukaemia (AML), chronic syndromes (MDS)).

Thus, the present invention also relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is administered in combination with one or more additional agents selected from:
(i) an anthracycline topoisomerase 2 inhibitors (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

The present invention also relates to the use of a METTL3 inhibitor as defined herein, or a pharmaceutically acceptable salt thereof, in the manufacture of a medicament for use in the treatment of cancer, wherein the METTL3 inhibitor as defined herein, or a pharmaceutically acceptable salt thereof, is administered in combination with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

The present invention also relates to a method of treating cancer, the method comprising administering a therapeutically effective amount of a METTL3 inhibitor as defined herein, or a pharmaceutically acceptable salt thereof, to a patient in combination with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

Suitably, the METTL3 inhibitor as defined herein, or a pharmaceutically acceptable salt thereof, is administered simultaneously, sequentially or separately with:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
or a pharmaceutically-acceptable salt thereof.

In one aspect the present invention relates to a combination comprising a METTL3 inhibitor as defined herein, or a pharmaceutically acceptable salt thereof, and one or more additional agents selected from:
(i) an anthracycline topoisomerase 2 inhibitors (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect the present invention relates to a pharmaceutical product comprising a combination as defined above.

In another aspect, the present invention relates to a pharmaceutical composition comprising a combination as defined above, and one or more pharmaceutically acceptable excipients.

In another aspect, the present invention relates to a combination as defined above, or a pharmaceutical product as defined above, or a pharmaceutical composition as defined above for use in therapy.

In another aspect, the present invention relates to a combination as defined above, or a pharmaceutical product as defined above, or a pharmaceutical composition as defined above for use in the treatment of cancer.

In another aspect, the present invention relates to the use of a combination as defined above in the manufacture of a medicament for the treatment of cancer.

In another aspect, the present invention relates to a method of treating of cancer in a subject in need thereof comprising administering to said subject a therapeutically effective amount of a combination as defined above.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect, the present invention relates to an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, for use in the treatment of cancer, wherein the agent, or a pharmaceutically acceptable salt thereof, is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect, the present invention relates to a use of an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof,
   wherein the METTL3 inhibitor as defined herein, or a pharmaceutically acceptable salt thereof, and the agent, or a pharmaceutically acceptable salt thereof, are administered sequentially, separately or simultaneously with one another.

In another aspect, the present invention relates to a method of treating cancer, or potentiating the effect of an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, the method comprising administering to a patient in need of such treatment a therapeutically effective amount the agent, or a pharmaceutically acceptable salt thereof, separately, sequentially or simultaneously with a METTL3 inhibitor as defined herein, or a pharmaceutically acceptable salt thereof.

In another aspect, the present invention relates to a method of treating cancer, or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

Suitably, the cancer is selected from acute myeloid leukaemia (AML), chronic syndromes (MDS), especially AML.

Preferred, suitable, and optional features of any one particular aspect of the present invention described herein are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

An “inhibitor” may be a polypeptide, nucleic acid, carbohydrate, lipid, small molecular weight compound, an oligonucleotide, an oligopeptide, siRNA, antisense, a recombinant protein, an antibody, a peptibody, or conjugates or fusion proteins thereof. For a review of siRNA see Milhavet O, Gary D S, Mattson M P. (Pharmacol Rev. 2003 December; 55(4):629-48. For a review of antisense see Opalinska J B, Gewirtz A M. Sci STKE. 2003 Oct. 28; 2003 (206): p47. A small molecular weight compound refers to a compound with a molecular weight of less than 2000 Daltons, less than 1000 Daltons, less than 700 Daltons or less than 500 Daltons.

References to “a pharmaceutically acceptable salt” of an inhibitor defined herein is refers to any salt form suitable for pharmaceutical use. Examples of pharmaceutically acceptable salts include an acid-addition salt of an inhibitor of the invention which is sufficiently basic, for example, an acid-addition salt with, for example, an inorganic or organic acid, for example hydrochloric, hydrobromic, sulfuric, phosphoric, trifluoracetic, formic, citric methane sulfonate or maleic acid. In addition, a suitable pharmaceutically acceptable salt of an inhibitor of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

References herein to a METTL3 inhibitor as defined herein, or a pharmaceutically acceptable salt thereof, and an immune checkpoint inhibitor, or a pharmaceutically acceptable salt thereof, include, where appropriate, any isomeric, tautomeric, polymorphic, amorphous and solvate (e.g. hydrate) forms of the inhibitors. An inhibitor may also be administered in the form of a prodrug which is broken down in the human or animal body to release the active inhibitor. Examples of pro-drugs include in vivo cleavable ester derivatives of the inhibitors that may be formed at a carboxy group or a hydroxy group in an inhibitor compound and in-vivo cleavable amide derivatives that may be formed at a carboxy group or an amino group in an inhibitor compound. Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

References herein to a METTL3 inhibitor as defined herein, or a pharmaceutically acceptable salt thereof, being administered “in combination with” another agent, or a pharmaceutically acceptable salt thereof, or vice versa, unless otherwise stated otherwise, include the inhibitors being administered sequentially, separately or simultaneously with one another.

As used herein “simultaneous administration” refers to therapy in which the both agents (e.g. a METTL3 inhibitor as defined herein, or a pharmaceutically acceptable salt thereof, and immune checkpoint inhibitor) are administered at the same time, suitably as a mono-therapy.

As used herein “sequential administration” means that one agent is administered after the other, however, the time period between the administration of each agent is such that both agents are capable of acting therapeutically concurrently. Thus, administration “sequentially” may permit one agent to be administered within seconds, minutes, or a matter of hours after the other provided the circulatory half-life of the first administered agent is such that they are both concurrently present in therapeutically effective amounts. The time delay between the administration of the agents may vary depending on the exact nature of the agents, the interaction there between, and their respective half-lives.

As used herein, “separate administration” means that one agent is administered after the other, however, the time period between administration is such that the first administered agent is no longer present a therapeutically effective amount when the second agent is administered. Accordingly, the two agents exert their therapeutic effects separately. Nevertheless, the overall therapeutic effect observed when the two agents separately act therapeutically may be greater than either agent used alone.

As used herein the, “subject(s)” and/or “patient(s)”, suitably refer to mammals (e.g. humans and non-human mammals such as livestock (cows, sheep, goats) or companion animals (cats, dogs, horses, rabbits). Suitably, the subject(s) and/or patient(s) are human(s).

As used herein, a “pharmaceutical product” refers to a product comprising a pharmaceutical. For instance, examples of a pharmaceutical product include a medical device, a pharmaceutical composition and a kit of parts suitably comprising one or more devices, containers and/or pharmaceuticals.

### Combination Therapies of the Present Invention

METTL3 inhibitors

The present invention resides in the recognition that METTL3 inhibitors are viable agents for use in combination with
(i) immuno-oncology agents (e.g. immune checkpoint inhibitors);
(ii) BCL2 inhibitors (e.g. venetoclax);
(iii) anthracycline topoisomerase 2 inhibitors (e.g. daunorubicin);
(iv) cytarabine;
(v) hypomethylating agents (e.g. 5-azacitidine or decitabine); and/or
(vi) FLT3 inhibitors (e.g. quizartinib).

Any suitable METTL3 inhibitor may be used in the combination therapies defined herein.

Examples of suitable METTL3 inhibitors include:
(i) compounds defined in International Patent Publication No. WO2020/050898, which have the generic formula I set out below;
(ii) the specific compounds of formula I set out in list 1 below;
(iii) compounds of the formulae (II), (VI) or (VII) defined below;
(iv) the specific compounds of formula II set out in List 2 below; or
(v) the specific compounds STM3006, STM3480 and STM3675 defined further below.
   (i) Compounds Defined in International Patent Publication No. WO2020/050898

The entire contents of International Patent Publication No. WO2020/050898 are incorporated herein by reference.

The compounds defined therein have the following formula (I):

X—Y—Z (I)
wherein:
X is selected from:

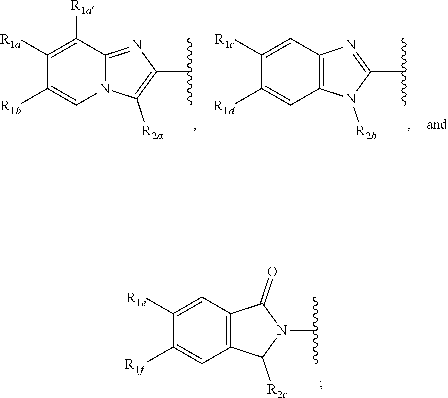

wherein:
R₁ₐ, R_{1b}, R_{1c}, R_{1d}, R₁ₑ and R_{1f} are independently selected from hydrogen, cyano, halo or a group of the formula:

-L₁ₐ-L_{1b}-Q₁
wherein
L₁ₐ is absent or selected from C₁₋₃alkylene and C₃₋₅cycloalkylene, wherein C₁₋₃alkylene and C₃₋₅cycloalkylene are optionally substituted by one or more substituents selected from aryl, aryl-(1-2C)alkyl, heteroaryl, aryl-(1-2C)alkyl, C₁₋₃alkyl, cyano, C₁₋₃alkoxy, halo, hydroxy, C₁₋₃haloalkoxy, —O—C₃₋₄cycloalkyl, NH₂ or oxo; wherein any —O—C₃₋₆cycloalkyl aryl, aryl-(1-2C)alkyl, heteroaryl, aryl-(1-2C)alkyl or C₁₋₃alkyl is optionally further substituted by one or more substituents selected from cyano, hydroxy, C₁₋₃alkoxy, halo, C₁₋₃haloalkoxy, —O—C₃₋₄cycloalkyl, or NH₂; wherein —O—C₃₋₆cycloalkyl is optionally further substituted with halo, cyano or hydroxy; or C₁₋₃ alkylene is optionally spiro-fused to a 3- to 5-membered cycloalkyl or heterocyclic ring, or a spirocyclic ring system, each of which is optionally substituted by one or more substituents selected from C₁₋₂alkyl, C₁₋₂haloalkyl, cyano, hydroxy, C₁₋₂alkoxy, halo or C₁₋₂haloalkoxy;
L_{1b} is absent or selected from O, S, SO, SO₂, N(Rᵣ), C(O), C(O)O, OC(O), C(O)N(Rᵣ), N(Rᵣ)C(O), N(Rᵣ)C(O)N(Rₛ), S(O)₂N(Rᵣ), or N(Rᵣ)SO₂, wherein Rᵣ and Rₛ are each independently selected from hydrogen or C₁₋₃alkyl, wherein C₁₋₃alkyl is optionally further substituted by cyano, hydroxy, C₁₋₂alkoxy, halo, C₁₋₂haloalkoxy, NH₂, C₃₋₆-cycloalkyl or a 3 to 6 membered heterocyclyl, wherein the C₃₋₆cycloalkyl or a 3 to 6 membered heterocyclyl in turn are optionally further substituted by halo, hydroxy, C₁₋₂alkoxy or C₁₋₂haloalkoxy; and
Q₁ is hydrogen, cyano, C₁₋₆alkyl, C₃₋₈cycloalkyl (including a spirocyclic carbocyclic and a bridged C₃₋₈cycloalkyl), C₂₋₃alkenyl, C₂₋₃alkynyl, aryl, heterocyclyl (including a mono- or bicyclic-heterocyclic ring system, a spirocyclic heterocyclic ring system, or a bridged heterocyclic ring system) or heteroaryl; and wherein Q₁ is optionally substituted by one or more substituents selected from C₁₋₄alkyl, halo, trifluoromethyl, trifluoromethoxy, amino, oxo, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, —S(O)₀₋₂RₜRᵤ, S(O)_{y}Rₜ (where y is 0, 1 or 2), SO₂N(Rₜ)Rᵤ, N(Rₜ)SO₂Rᵤ or (CH₂)₂NRₜRᵤ (where z is 1, 2 or 3), wherein C₁₋₄alkyl is in turn optionally substituted by one more substituents selected from cyano, hydroxy, C₁₋₂alkoxy, halo, C₁₋₂haloalkoxy, —O—C₃cycloalkyl, wherein —O—C₃cycloalkyl is optionally substituted with halo, cyano or hydroxy; and wherein Rₜ and Rᵤ are each independently selected from hydrogen or C₁₋₄alkyl; or
Q₁ is optionally substituted by one or more groups of the formula:

-L_{1c}-L_{1d}-Z₁
wherein
L_{1c} is absent or C₁₋₃alkylene optionally substituted by C₁₋₂alkyl or oxo;
L_{1d} is absent or selected from C(O), O, C(O)O, OC(O), C(O)N(Rᵥ), N(Rᵥ)C(O), N(Rᵥ)C(O)N(R_{w}), S(O)₂N(Rᵥ), or N(Rᵥ)SO₂, wherein Rᵥ and R_{w} are each independently selected from hydrogen or C₁₋₂alkyl; and
Z₁ is C₃₋₈cycloalkyl (including a spirocyclic carbocyclic and a bridged C₃₋₈cycloalkyl), heterocyclyl (including a mono- or bicyclic-heterocyclic ring system, a spirocyclic heterocyclic ring system, or a bridged heterocyclic ring system), aryl or heteroaryl, wherein Z₁ is optionally substituted by one or more substituents selected from C₁₋₄alkyl, C₃₋₆cycloalkyl, heterocyclyl, halo, C₁₋₄haloalkyl, C₁₋₄haloalkoxy, C₁₋₄alkoxy, cyano, hydroxyl, NRₜ₁Rᵤ₁, ORₜ₁, C(O)Rₜ₁, C(O)ORₜ₁, OC(O)Rₜ₁, C(O)N(Rₜ₁)Rᵤ₁, N(Rₜ₁)C(O)Rᵤ₁, —S(O)₀₋₂Rₜ₁Rᵤ₁, S(O)_{y}Rₜ₁ (where y is 0, 1 or 2), SO₂N(Rₜ₁)Rᵤ₁, N(Rₜ₁)SO₂Rᵤ₁ or (CH₂)₂NRₜ₁Rᵤ₁ (where z is 1, 2 or 3), wherein Rₜ₁ and Rᵤ₁ are each independently selected from hydrogen or C₁₋₄alkyl; and when Z₁ is C₃₋₈cycloalkyl or heterocyclyl, Z₁ is optionally spiro-fused to a C₃₋₆cycloalkyl or heterocyclyl ring;
R_{1a′} is selected from hydrogen, halo and methyl;
R₂ₐ, R_{2b} and R_{2c} are selected from hydrogen, halo or a group of the formula:

-L₂ₐ-L_{2b}-Q₂

wherein
L₂ₐ is absent or C₁₋₃alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂alkyl; and
Q₂ is hydrogen, cyano, C₁₋₆alkyl, C₃₋₆cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)₂NRₚR_{q} (where z is 1, 2 or 3), wherein Rₚ and R_{q} are each independently selected from hydrogen or C₁₋₄alkyl;
Y is selected from:

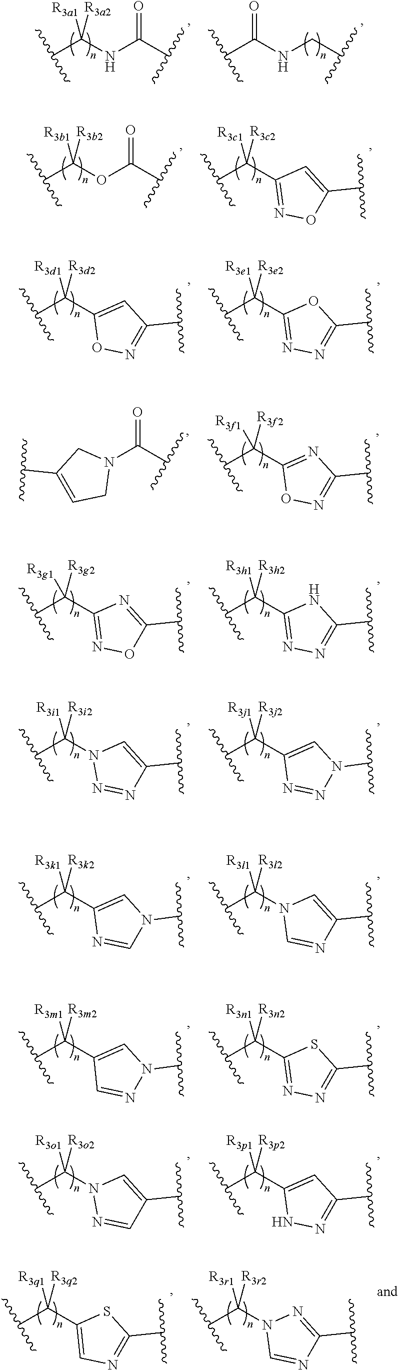

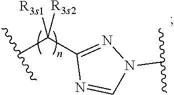

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆alkyl, C₃₋₄ cycloalkyl, hydroxy, and halo; and wherein C₁₋₆alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, Or R₃ᵣ₁ and R₃ᵣ₂ or R₃ₛ₁ and R₃ₛ₂ may be linked such that, together with the carbon atom to which they are attached, they form a spiro-fused C₃₋₄cycloalkyl which is optionally substituted with one or more substituents selected from halo, methyl, amino, cyano, and hydroxy;
   Z is selected from:

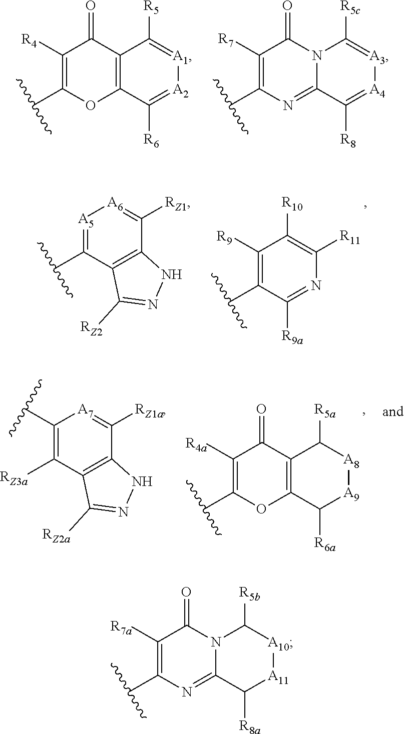

wherein:
R₄, R₇, R₄ₐ and R₇ₐ are independently selected from hydrogen, halo, cyano and methyl;
R₅, R₅ₐ, R_{5b} and R_{5c} are independently selected from hydrogen, halo, cyano and methyl;
R₆, R₈, R₆ₐ and R₈ₐ are independently selected from hydrogen, halo, cyano and methyl;
R₉, R₉ₐ, R₁₀ and R₁₁ are independently selected from hydrogen, NH₂, halo, cyano, and C₁₋₆ alkyl; or
R₉ and R₁₀ may be linked together to form a fused 5- or 6-membered saturated or unsaturated ring system or R₁₀ and R₁₁ may be linked together to form a fused 5- or 6-membered saturated or unsaturated ring system; wherein either of the fused 5- or 6-membered saturated or unsaturated ring system may be optionally substituted by one or more substituents selected from C₁₋₂alkyl, cyano, C₁₋₂haloalkyl, hydroxy, C₁₋₂alkoxy, halo, C₁₋₂haloalkoxy, NR₁ᵢₐR₁ⱼₐ or —S(O)₀₋₂R₁ᵢₐR₁ⱼₐ, wherein R₁ᵢₐ and R₁ⱼₐ are H or C₁₋₂alky;
R_{Z1} and R_{Z1a} are selected from hydrogen, C₁₋₄alkyl, cyano, halo, C₁₋₄haloalkyl, C₁₋₄haloalkoxy, C₁₋₄alkoxy, C₃₋₆cycloalkyl and —O—C₃₋₆cycloalkyl, wherein C₃₋₆cycloalkyl and —O—C₃₋₆cycloalkyl are optionally substituted by one or more of halo, methyl or methoxy;
R_{Z2} and R_{Z2a} are selected from hydrogen, C₁₋₄alkyl, cyano, halo, NH₂ and C₁₋₄alkoxy;
R_{Z3a} is selected from hydrogen, C₁₋₄alkyl, cyano, halo, NH₂ and C₁₋₄alkoxy
A₁ is selected from CR₁₂ and N;
A₂ is selected from CR₁₃ and N;
A₃ is selected from CR₁₄ and N;
A₄ is selected from CR₁₅ and N;
A₅ is selected from CR₁₆ and N;
A₆ is selected from CR₁₇ and N;
A₇ is selected from CR₁₈ and N;
A₈ is selected from CR₁₉R₂₀ and NR₂₁;
A₉ is selected from CR₂₂R₂₃ and NR₂₄;
A₁₀ is selected from CR₂₅R₂₆ and NR₂₇;
A₁₁ is selected from CR₂₈R₂₉ and NR₃₀;
R₁₂ and R₁₄ are independently selected from hydrogen, halo, cyano and C₁₋₄ alkyl;
R₁₃ is selected from hydrogen, halo, cyano, methoxy and methyl;
R₁₅ is selected from hydrogen, halo, cyano methoxy and methyl;
R₁₆ is selected from hydrogen, halo, cyano, C₁₋₄ alkyl, C₁₋₄ alkoxy, C₁₋₄haloalkyl, C₁₋₄haloalkoxy, C₃₋₄cycloalkyl, a 3- to 4-membered heterocyclyl and C₃₋₄cycloalkoxy;
R₁₇ is selected from hydrogen, hydroxy, halo, cyano, C₁₋₅ alkyl, C₁₋₄ haloalkyl, C₁₋₄alkoxy, C₁₋₄ haloalkoxy, C₂₋₄ alkenyl, C₂₋₄ alkynyl, phenyl, a 5- or 6-membered or heteroaryl, C₃₋₆cycloalkyl, —O—C₃₋₆cycloalkyl, heterocyclyl, —O-heterocyclyl (carbon-linked), —(OCH₂CH₂)ₘ—NR_{q}Rᵣ, —(OCH₂CH₂)ₘ—OCH₃ wherein m is an integer from 1 to 6, NR_{q}Rᵣ, —C(O)—NR_{q}Rᵣ, —C(O)OR_{q};
wherein R_{q} and Rᵣ are each independently hydrogen, C₁₋₅ alkyl, C₃₋₆cycloalkyl, a 3- to 6-membered carbon-linked heterocyclyl, wherein C₁₋₅ alkyl, C₃₋₆cycloalkyl, a 3- to 6-membered carbon-linked heterocyclyl may be optionally substituted by one or more substituents selected from C₁₋₂alkyl, cyano, C₁₋₂haloalkyl, hydroxy, C₁₋₂alkoxy, halo, C₁₋₂haloalkoxy, NR₁ₑₐR_{1fa} or —S(O)₀₋₂R₁ₑₐR_{1fa}, wherein R₁ₑₐ and R_{1fa} are H or C₁₋₂alkyl;
or R_{q} and Rᵣ are linked together such that, together with the nitrogen atom to which they are attached, they form a 3- to 6-membered heterocyclic ring, which may be optionally substituted by one or more substituents selected from C₁₋₂alkyl, cyano, C₁₋₂haloalkyl, hydroxy, C₁₋₂alkoxy, halo, C₁₋₂haloalkoxy;
wherein any C₁₋₅alkyl, C₁₋₄ alkoxy, C₂₋₄alkenyl, C₂₋₄alkynyl, phenyl, 5- or 6-membered or heteroaryl, C₃₋₆cycloalkyl, —O—C₃₋₆cycloalkyl, heterocyclyl or —O-heterocyclyl (carbon-linked) is optionally further substituted by one or more substituents selected from C₁₋₂alkyl, cyano, C₁₋₂haloalkyl, hydroxy, C₁₋₂alkoxy, halo, C₁₋₂haloalkoxy, NR₁ₑₐR_{1fa} or —S(O)₀₋₂R₁ₑₐR_{1fa}, wherein R₁ₑₐ and R_{1fa} are H or C₁₋₂alkyl;
R₁₈ is selected from hydrogen, halo, cyano, C₁₋₄ alkyl, C₂₋₄ alkenyl, C₂₋₄ alkynyl, a 5- or 6-membered heteroaryl, C₁₋₄ alkoxy, C₁₋₄haloalkyl, C₁₋₄haloalkoxy, C₃₋₄cycloalkyl, a 3- to 4-membered heterocyclyl and C₃₋₄cycloalkoxy;
R₁₉, R₂₀, R₂₅ and R₂₆ are selected from hydrogen, halo, cyano and C₁₋₄ alkyl;
R₂₂ and R₂₃ are selected from hydrogen, halo, cyano and methyl;
R₂₈ and R₂₉ are selected from hydrogen, methoxy and methyl;
R₂₁, R₂₄, R₂₇ and R₃₀ are hydrogen; and
n is 0, 1 or 2;
with the proviso that the compound is not:
2-((1H-benzo[d]imidazol-2-yl)methyl)-5-(pyridin-3-yl)-1,3,4-oxadiazole;
2-((6-chloro-1-phenyl-1H-benzo[d]imidazol-2-yl)methyl)-5-(pyridin-3-yl)-1,3,4-oxadiazole;
N-({6-methylimidazo[1,2-a]pyridin-2-yl}methyl)-1H-indazole-5-carboxamide.

### (ii) Specific Compounds of Formula I—List 1

Particular compounds of formula I disclosed in International Patent Publication No. WO2020/050898 include any of the following in List 1 below, or a pharmaceutically acceptable salt thereof:

### List 1

N-({6-methylimidazo[1,2-a]pyridin-2-yl}methyl)-1H-indazole-4-carboxamide;
N-({7-bromoimidazo[1,2-a]pyridin-2-yl}methyl)-1H-indazole-4-carboxamide;
N-({6-bromoimidazo[1,2-a]pyridin-2-yl}methyl)-1H-indazole-4-carboxamide;
N-({6-chloroimidazo[1,2-a]pyridin-2-yl}methyl)-1H-indazole-4-carboxamide;
N-({7-fluoroimidazo[1,2-a]pyridin-2-yl}methyl)-1H-indazole-4-carboxamide;
N-({6-fluoroimidazo[1,2-a]pyridin-2-yl}methyl)-1H-indazole-4-carboxamide;
N-({7-methoxyimidazo[1,2-a]pyridin-2-yl}methyl)-1H-indazole-4-carboxamide;
N-({6-methylimidazo[1,2-a]pyridin-2-yl}methyl)-6-(1H-pyrazol-5-yl)-1H-indazole-4-carboxamide;
N-({7-methylimidazo[1,2-a]pyridin-2-yl}methyl)-1H-indazole-4-carboxamide;
6-bromo-N-({6-methylimidazo[1,2-a]pyridin-2-yl}methyl)-1H-indazole-4-carboxamide;
6-bromo-N-({7-methylimidazo[1,2-a]pyridin-2-yl}methyl)-1H-indazole-4-carboxamide;
N-({6-methylimidazo[1,2-a]pyridin-2-yl}methyl)-4-oxo-4H-chromene-2-carboxamide;
N-({imidazo[1,2-a]pyridin-2-yl}methyl)-4-oxo-4H-chromene-2-carboxamide;
6-ethynyl-N-({6-methylimidazo[1,2-a]pyridin-2-yl}methyl)-1H-indazole-4-carboxamide;
N-({6-methylimidazo[1,2-a]pyridin-2-yl}methyl)-4-oxo-4H-pyrido[1,2-a]pyrimidine-2-carboxamide;
N-({7-methylimidazo[1,2-a]pyridin-2-yl}methyl)-4-oxo-4H-chromene-2-carboxamide;
N-({7-methylimidazo[1,2-a]pyridin-2-yl}methyl)-4-oxo-4H-pyrido[1,2-a]pyrimidine-2-carboxamide;
N-({6-cyanoimidazo[1,2-a]pyridin-2-yl}methyl)-4-oxo-4H-pyrido[1,2-a]pyrimidine-2-carboxamide;
8-methoxy-N-({6-methylimidazo[1,2-a]pyridin-2-yl}methyl)-4-oxo-4H-pyrido[1,2-a]pyrimidine-2-carboxamide;
N-({6-methylimidazo[1,2-a]pyridin-2-yl}methyl)-1H-indazole-5-carboxamide;
7-chloro-N-({6-methylimidazo[1,2-a]pyridin-2-yl}methyl)-4-oxo-4H-pyrido[1,2-a]pyrimidine-2-carboxamide;
N-({6-fluoroimidazo[1,2-a]pyridin-2-yl}methyl)-4-oxo-4H-pyrido[1,2-a]pyrimidine-2-carboxamide;
N-({6-bromoimidazo[1,2-a]pyridin-2-yl}methyl)-4-oxo-4H-pyrido[1,2-a]pyrimidine-2-carboxamide;
N-({6-methoxyimidazo[1,2-a]pyridin-2-yl}methyl)-4-oxo-4H-pyrido[1,2-a]pyrimidine-2-carboxamide;
N-({7-methoxyimidazo[1,2-a]pyridin-2-yl}methyl)-4-oxo-4H-pyrido[1,2-a]pyrimidine-2-carboxamide;
N-({imidazo[1,2-a]pyridin-2-yl}methyl)-4-oxo-4H-pyrido[1,2-a]pyrimidine-2-carboxamide;
N-({8-methylimidazo[1,2-a]pyridin-2-yl}methyl)-4-oxo-4H-pyrido[1,2-a]pyrimidine-2-carboxamide;
N-({6-methylimidazo[1,2-a]pyridin-2-yl}methyl)-1H-pyrazolo[4,3-c]pyridine-4-carboxamide;
N-({7-bromoimidazo[1,2-a]pyridin-2-yl}methyl)-4-oxo-4H-pyrido[1,2-a]pyrimidine-2-carboxamide;
N-(2-hydroxy-1-{6-methylimidazo[1,2-a]pyridin-2-yl}ethyl)-1H-indazole-4-carboxamide;
4-(3-{imidazo[1,2-a]pyridin-2-yl}-2,5-dihydro-1H-pyrrole-1-carbonyl)-1H-indazole;
N-[(6-{[(pyridin-3-yl)methyl]amino}imidazo[1,2-a]pyridin-2-yl)methyl]-1H-indazole-4-carboxamide;
N-[(6-{[(1-methyl-1H-imidazol-4-yl)methyl]amino}imidazo[1,2a]pyridin-2-yl)methyl]-1H-indazole-4-carboxamide;
N-{[6-(3-methoxyphenyl)imidazo[1,2-a]pyridin-2-yl]methyl}-1H-indazole-4-carboxamide;
N-{[6-(1H-pyrazol-5-yl)imidazo[1,2-a]pyridin-2-yl]methyl}-1H-indazole-4-carboxamide;
N-[[6-(3-chlorophenyl)imidazo[1,2-a]pyridin-2-yl]methyl]-1H-indazole-4-carboxamide;
N-({6-[(pyridin-3-yl)amino]imidazo[1,2-a]pyridin-2-yl}methyl)-1H-indazole-4-carboxamide;
N-({6-[(piperazin-1-yl)methyl]imidazo[1,2-a]pyridin-2-yl}methyl)-1H-indazole-4-carboxamide;
N-{[6-(aminomethyl)imidazo[1,2-a]pyridin-2-yl]methyl}-1H-indazole-4-carboxamide;
N-{[6-(acetamidomethyl)imidazo[1,2-a]pyridin-2-yl]methyl}-1H-indazole-4-carboxamide;
{6-methylimidazo[1,2-a]pyridin-2-yl}methyl 1H-indazole-4-carboxylate;
{6-methylimidazo[1,2-a]pyridin-2-yl}methyl 1H-indazole-4-carboxylate hydrochloride;
N-{[6-(hydroxymethyl)imidazo[1,2-a]pyridin-2-yl]methyl}-1H-indazole-4-carboxamide;
N-({6-hydroxyimidazo[1,2-a]pyridin-2-yl}methyl)-1H-indazole-4-carboxamide;
N-({6-[(methylamino)methyl]imidazo[1,2-a]pyridin-2-yl}methyl)-1H-indazole-4-carboxamide;
N-({6-[(methylamino)methyl]imidazo[1,2-a]pyridin-2-yl}methyl)-1H-indazole-4-carboxamide dihydrochloride;
N-[(6-{[(2-hydroxyethyl)amino]methyl}imidazo[1,2-a]pyridin-2-yl)methyl]-1H-indazole-4-carboxamide;
N-[(6-{[(2,2,2-trifluoroethyl)amino]methyl}imidazo[1,2-a]pyridin-2-yl)methyl]-1H-indazole-4-carboxamide;
N-{[6-(1-hydroxyethyl)imidazo[1,2-a]pyridin-2-yl]methyl}-1H-indazole-4-carboxamide;
N-({6-[hydroxy(phenyl)methyl]imidazo[1,2-a]pyridin-2-yl}methyl)-1H-indazole-4-carboxamide;
N-({6-formylimidazo[1,2-a]pyridin-2-yl}methyl)-4-oxo-4H-pyrido[1,2-a]pyrimidine-2-carboxamide;
N-{[6-(aminomethyl)imidazo[1,2-a]pyridin-2-yl]methyl}-4-oxo-4H-pyrido[1,2-a]pyrimidine-2-carboxamide;
4-oxo-N-[(6-{[(2,2,2-trifluoroethyl)amino]methyl}imidazo[1,2-a]pyridin-2-yl)methyl]-4H-pyrido[1,2-a]pyrimidine-2-carboxamide;
N-[(6-{[(2-hydroxyethyl)amino]methyl}imidazo[1,2-a]pyridin-2-yl)methyl]-4-oxo-4H-pyrido[1,2-a]pyrimidine-2-carboxamide;
4-oxo-N-[(6-{[(2-phenylethyl)amino]methyl}imidazo[1,2-a]pyridin-2-yl)methyl]-4H-pyrido[1,2-a]pyrimidine-2-carboxamide;
N-({6-[(benzylamino)methyl]imidazo[1,2-a]pyridin-2-yl}methyl)-4-oxo-4H-pyrido[1,2-a]pyrimidine-2-carboxamide;
4-oxo-N-[(6-{[(3-phenylpropyl)amino]methyl}imidazo[1,2-a]pyridin-2-yl)methyl]-4H-pyrido[1,2-a]pyrimidine-2-carboxamide;
N-({6-[hydroxy(phenyl)methyl]imidazo[1,2-a]pyridin-2-yl}methyl)-4-oxo-4H-pyrido[1,2-a]pyrimidine-2-carboxamide;
N-{[6-(1-hydroxyethyl)imidazo[1,2-a]pyridin-2-yl]methyl}-4-oxo-4H-pyrido[1,2-a]pyrimidine-2-carboxamide;
N-({6-ethenylimidazo[1,2-a]pyridin-2-yl}methyl)-4-oxo-4H-pyrido[1,2-a]pyrimidine-2-carboxamide;
N-({6-cyclopropylimidazo[1,2-a]pyridin-2-yl}methyl)-4-oxo-4H-pyrido[1,2-a]pyrimidine-2-carboxamide;
N-({7-ethenylimidazo[1,2-a]pyridin-2-yl}methyl)-4-oxo-4H-pyrido[1,2-a]pyrimidine-2-carboxamide;
N-{[6-(hydroxymethyl)imidazo[1,2-a]pyridin-2-yl]methyl}-4-oxo-4H-pyrido[1,2-a]pyrimidine-2-carboxamide;
2-(1H-indazol-4-yl)-5-[(6-methylimidazo[1,2-a]pyridin-2-yl)methyl]-1,3,4-oxadiazole;
N-{[6-(2-aminoethyl)imidazo[1,2-a]pyridin-2-yl]methyl}-4-oxo-4H-pyrido[1,2-a]pyrimidine-2-carboxamide;
N-({6-methylimidazo[1,2-a]pyridin-2-yl}methyl)-4-oxo-4H, 6H, 7H,8H,9H-pyrido[1,2-a]pyrimidine-2-carboxamide;
4-oxo-N-{[6-(trifluoromethyl)imidazo[1,2-a]pyridin-2-yl]methyl}-4H-pyrido[1,2-a]pyrimidine-2-carboxamide;
tert-butyl N-(2-{2-[({4-oxo-4H-pyrido[1,2-a]pyrimidin-2-yl}formamido)methyl]imidazo[1,2-a]pyridin-6-yl}ethyl)carbamate;
N-benzyl-2-[({4-oxo-4H-pyrido[1,2-a]pyrimidin-2-yl}formamido)methyl]imidazo[1,2-a]pyridine-6-carboxamide;
N-[(6-{[(cyclohexylmethyl)amino]methyl}imidazo[1,2-a]pyridin-2-yl)methyl]-4-oxo-4H-chromene-2-carboxamide;
7-chloro-N-[(6-{[(cyclohexylmethyl)amino]methyl}imidazo[1,2-a]pyridin-2-yl)methyl]-4-oxo-4H-chromene-2-carboxamide;
6-chloro-N-[(6-{[(cyclohexylmethyl)amino]methyl}imidazo[1,2-a]pyridin-2-yl)methyl]-4-oxo-4H-chromene-2-carboxamide;
N-[(6-{[(cyclohexylmethyl)amino]methyl}imidazo[1,2-a]pyridin-2-yl)methyl]-4-oxo-4H,6H,7H,8H,9H-pyrido[1,2-a]pyrimidine-2-carboxamide;
6-amino-N-[(6-{[(cyclohexylmethyl)amino]methyl}imidazo[1,2-a]pyridin-2-yl)methyl]pyridine-3-carboxamide;
N-({6-[(benzyloxy)methyl]imidazo[1,2-a]pyridin-2-yl}methyl)-4-oxo-4H-pyrido[1,2-a]pyrimidine-2-carboxamide;
N-({6-[(benzylamino)methyl]imidazo[1,2-a]pyridin-2-yl}methyl)-1H-indazole-4-carboxamide;
N-[(6-{[(cyclohexylmethyl)amino]methyl}imidazo[1,2-a]pyridin-2-yl)methyl]-7-fluoro-4-oxo-4H-chromene-2-carboxamide;
N-[(6-{[(cyclohexylmethyl)amino]methyl}imidazo[1,2-a]pyridin-2-yl)methyl]-7-methyl-4-oxo-4H-chromene-2-carboxamide;
N-[(6-{[(cyclohexylmethyl)amino]methyl}imidazo[1,2-a]pyridin-2-yl)methyl]-1H-indazole-4-carboxamide;
8-chloro-N-[(6-{[(cyclohexylmethyl)amino]methyl}imidazo[1,2-a]pyridin-2-yl)methyl]-4-oxo-4H-chromene-2-carboxamide;
6-bromo-N-[(6-{[(cyclohexylmethyl)amino]methyl}imidazo[1,2-a]pyridin-2-yl)methyl]-1H-indazole-4-carboxamide;
N-[(6-{[(cyclohexylmethyl)amino]methyl}imidazo[1,2-a]pyridin-2-yl)methyl]quinoline-3-carboxamide;
N-[(6-{1-[(cyclohexylmethyl)amino]ethyl}imidazo[1,2-a]pyridin-2-yl)methyl]-4-oxo-4H-pyrido[1,2-a]pyrimidine-2-carboxamide;
6-chloro-N-[(6-{[(cyclohexylmethyl)amino]methyl}imidazo[1,2-a]pyridin-2-yl)methyl]-1H-indazole-4-carboxamide;
4-[5-({6-methylimidazo[1,2-a]pyridin-2-yl}methyl)-1,3,4-thiadiazol-2-yl]-1H-indazole; 4-[1-({6-methylimidazo[1,2-a]pyridin-2-yl}methyl)-1H-1,2,3-triazol-4-yl]-1H-indazole;
N-[(6-{[(3-chlorophenyl)amino]methyl}imidazo[1,2-a]pyridin-2-yl)methyl]-4-oxo-4H-pyrido[1,2-a]pyrimidine-2-carboxamide;
N-[(6-{[(1-cyclohexyl-2-hydroxyethyl)amino]methyl}imidazo[1,2-a]pyridin-2-yl)methyl]-4-oxo-4H-pyrido[1,2-a]pyrimidine-2-carboxamide;
4-oxo-N-{[6-({[(pyridin-3-yl)methyl]amino}methyl)imidazo[1,2-a]pyridin-2-yl]methyl}-4H-pyrido[1,2-a]pyrimidine-2-carboxamide;
N-{[6-({[(4-methoxyphenyl)methyl]amino}methyl)imidazo[1,2-a]pyridin-2-yl]methyl}-4-oxo-4H-pyrido[1,2-a]pyrimidine-2-carboxamide;
N-[(6-{[(cyclohexylmethyl)amino]methyl}imidazo[1,2-a]pyridin-2-yl)methyl]-4-oxo-4H-pyrido[1,2-a]pyrimidine-2-carboxamide;
N-{[6-({[(4-chlorophenyl)methyl]amino}methyl)imidazo[1,2-a]pyridin-2-yl]methyl}-4-oxo-4H-pyrido[1,2-a]pyrimidine-2-carboxamide;
N-[(6-{[benzyl(methyl)amino]methyl}imidazo[1,2-a]pyridin-2-yl)methyl]-4-oxo-4H-pyrido[1,2-a]pyrimidine-2-carboxamide;
4-oxo-N-{[6-({[(1R)-1-phenylethyl]amino}methyl)imidazo[1,2-a]pyridin-2-yl]methyl}-4H-pyrido[1,2-a]pyrimidine-2-carboxamide;
4-oxo-N-{[6-({[(1S)-1-phenylethyl]amino}methyl)imidazo[1,2-a]pyridin-2-yl]methyl}-4H-pyrido[1,2-a]pyrimidine-2-carboxamide;
N-{[6-({[(2-fluorophenyl)methyl]amino}methyl)imidazo[1,2-a]pyridin-2-yl]methyl}-4-oxo-4H-pyrido[1,2-a]pyrimidine-2-carboxamide;
4-oxo-N-[(6-{[(2-phenylpropan-2-yl)amino]methyl}imidazo[1,2-a]pyridin-2-yl)methyl]-4H-pyrido[1,2-a]pyrimidine-2-carboxamide;
N-{[6-({[(3-fluorophenyl)methyl]amino}methyl)imidazo[1,2-a]pyridin-2-yl]methyl}-4-oxo-4H-pyrido[1,2-a]pyrimidine-2-carboxamide;
N-{[6-({[(4-fluorophenyl)methyl]amino}methyl)imidazo[1,2-a]pyridin-2-yl]methyl}-4-oxo-4H-pyrido[1,2-a]pyrimidine-2-carboxamide;
4-oxo-N-[(6-{[(4,4,4-trifluorobutyl)amino]methyl}imidazo[1,2-a]pyridin-2-yl)methyl]-4H-pyrido[1,2-a]pyrimidine-2-carboxamide;
N-{[6-({[(oxan-4-yl)methyl]amino}methyl)imidazo[1,2-a]pyridin-2-yl]methyl}-4-oxo-4H-pyrido[1,2-a]pyrimidine-2-carboxamide;
N-{[6-({[(3,3-difluorocyclobutyl)methyl]amino}methyl)imidazo[1,2-a]pyridin-2-yl]methyl}-4-oxo-4H-pyrido[1,2-a]pyrimidine-2-carboxamide;
N-[(6-{[(cyclopropylmethyl)amino]methyl}imidazo[1,2-a]pyridin-2-yl)methyl]-4-oxo-4H-pyrido[1,2-a]pyrimidine-2-carboxamide;
4-oxo-N-[(6-{[(3,3,3-trifluoropropyl)amino]methyl}imidazo[1,2-a]pyridin-2-yl)methyl]-4H-pyrido[1,2-a]pyrimidine-2-carboxamide;
N-({6-[(cyclohexylamino)methyl]imidazo[1,2-a]pyridin-2-yl}methyl)-4-oxo-4H-pyrido[1,2-a]pyrimidine-2-carboxamide;
4-oxo-N-({6-[({[3-(trifluoromethyl)bicyclo[1.1.1]pentan-1-yl]methyl}amino)methyl]imidazo[1,2-a]pyridin-2-yl}methyl)-4H-pyrido[1,2-a]pyrimidine-2-carboxamide;
N-{[6-({[(oxan-2-yl)methyl]amino}methyl)imidazo[1,2-a]pyridin-2-yl]methyl}-4-oxo-4H-pyrido[1,2-a]pyrimidine-2-carboxamide;
4-oxo-N-[(6-{[(3-phenyloxetan-3-yl)amino]methyl}imidazo[1,2-a]pyridin-2-yl)methyl]-4H-pyrido[1,2-a]pyrimidine-2-carboxamide;
N-{[6-({[(oxan-3-yl)methyl]amino}methyl)imidazo[1,2-a]pyridin-2-yl]methyl}-4-oxo-4H-pyrido[1,2-a]pyrimidine-2-carboxamide;
N-{[6-({[(1-fluorocyclohexyl)methyl]amino}methyl)imidazo[1,2-a]pyridin-2-yl]methyl}-4-oxo-4H-pyrido[1,2-a]pyrimidine-2-carboxamide;
N-{[6-({[(3-cyclopropylphenyl)methyl]amino}methyl)imidazo[1,2-a]pyridin-2-yl]methyl}-4-oxo-4H-pyrido[1,2-a]pyrimidine-2-carboxamide;
N-[(6-{[(3,3-dimethylbutyl)amino]methyl}imidazo[1,2-a]pyridin-2-yl)methyl]-4-oxo-4H-pyrido[1,2-a]pyrimidine-2-carboxamide;
4-oxo-N-{[6-({[2-(trifluoromethoxy)ethyl]amino}methyl)imidazo[1,2-a]pyridin-2-yl]methyl}-4H-pyrido[1,2-a]pyrimidine-2-carboxamide;
4-oxo-N-{[6-({[(oxolan-2-yl)methyl]amino}methyl)imidazo[1,2-a]pyridin-2-yl]methyl}-4H-pyrido[1,2-a]pyrimidine-2-carboxamide;
N-[(6-{[(2-methanesulfonylethyl)amino]methyl}imidazo[1,2-a]pyridin-2-yl)methyl]-4-oxo-4H-pyrido[1,2-a]pyrimidine-2-carboxamide;
4-oxo-N-({6-[(3-phenylpyrrolidin-1-yl)methyl]imidazo[1,2-a]pyridin-2-yl}methyl)-4H-pyrido[1,2-a]pyrimidine-2-carboxamide;
N-[(6-{[(1-cyclohexylcyclopropyl)amino]methyl}imidazo[1,2-a]pyridin-2-yl)methyl]-4-oxo-4H-pyrido[1,2-a]pyrimidine-2-carboxamide;
4-oxo-N-{[6-({[(1,3-thiazol-5-yl)methyl]amino}methyl)imidazo[1,2-a]pyridin-2-yl]methyl}-4H-pyrido[1,2-a]pyrimidine-2-carboxamide;
tert-butyl 3-{[({2-[({4-oxo-4H-pyrido[1,2-a]pyrimidin-2-yl}formamido)methyl]imidazo[1,2-a]pyridin-6-yl}methyl)amino]methyl}piperidine-1-carboxylate;
N-{[6-({[(4,4-difluorocyclohexyl)methyl]amino}methyl)imidazo[1,2-a]pyridin-2-yl]methyl}-4-oxo-4H-pyrido[1,2-a]pyrimidine-2-carboxamide;
tert-butyl 2-{[({2-[({4-oxo-4H-pyrido[1,2-a]pyrimidin-2-yl}formamido)methyl]imidazo[1,2-a]pyridin-6-yl}methyl)amino]methyl}piperidine-1-carboxylate;
N-[(6-{[(2-cyclopropylethyl)amino]methyl}imidazo[1,2-a]pyridin-2-yl)methyl]-4-oxo-4H-pyrido[1,2-a]pyrimidine-2-carboxamide;
4-oxo-N-({6-[(piperidin-1-yl)methyl]imidazo[1,2-a]pyridin-2-yl}methyl)-4H-pyrido[1,2-a]pyrimidine-2-carboxamide;
4-oxo-N-({6-[({[1-(2,2,2-trifluoroethyl)-1H-pyrazol-3-yl]methyl}amino)methyl]imidazo[1,2-a]pyridin-2-yl}methyl)-4H-pyrido[1,2-a]pyrimidine-2-carboxamide;
N-{[6-({[(1-methylcyclohexyl)methyl]amino}methyl)imidazo[1,2-a]pyridin-2-yl]methyl}-4-oxo-4H-pyrido[1,2-a]pyrimidine-2-carboxamide;
4-oxo-N-{[6-({[2-(pyridin-3-yl)ethyl]amino}methyl)imidazo[1,2-a]pyridin-2-yl]methyl}-4H-pyrido[1,2-a]pyrimidine-2-carboxamide;
N-{[6-({[(1,4-dioxan-2-yl)methyl]amino}methyl)imidazo[1,2-a]pyridin-2-yl]methyl}-4-oxo-4H-pyrido[1,2-a]pyrimidine-2-carboxamide;
N-({6-[(cyclopropylamino)methyl]imidazo[1,2-a]pyridin-2-yl}methyl)-4-oxo-4H-pyrido[1,2-a]pyrimidine-2-carboxamide;
N-[(6-{[(3,3-difluorocyclobutyl)amino]methyl}imidazo[1,2-a]pyridin-2-yl)methyl]-4-oxo-4H-pyrido[1,2-a]pyrimidine-2-carboxamide;
N-{[6-({[(oxetan-3-yl)methyl]amino}methyl)imidazo[1,2-a]pyridin-2-yl]methyl}-4-oxo-4H-pyrido[1,2-a]pyrimidine-2-carboxamide;
4-oxo-N-{[6-({[(1R,2R)-2-(trifluoromethyl)cyclopropyl]amino}methyl)imidazo[1,2-a]pyridin-2-yl]methyl}-4H-pyrido[1,2-a]pyrimidine-2-carboxamide;
N-[(6-{[(2,2-dimethylpropyl)amino]methyl}imidazo[1,2-a]pyridin-2-yl)methyl]-4-oxo-4H-pyrido[1,2-a]pyrimidine-2-carboxamide;
N-[(6-{[(cyclohexylmethyl)(methyl)amino]methyl}imidazo[1,2-a]pyridin-2-yl)methyl]-4-oxo-4H-pyrido[1,2-a]pyrimidine-2-carboxamide;
N-[(6-{[(4,4-difluorocyclohexyl)amino]methyl}imidazo[1,2-a]pyridin-2-yl)methyl]-4-oxo-4H-pyrido[1,2-a]pyrimidine-2-carboxamide;
N-({6-[({bicyclo[1.1.1]pentan-1-yl}amino)methyl]imidazo[1,2-a]pyridin-2-yl}methyl)-4-oxo-4H-pyrido[1,2-a]pyrimidine-2-carboxamide;
4-oxo-N-({6-[({[(2S)-oxolan-2-yl]methyl}amino)methyl]imidazo[1,2-a]pyridin-2-yl}methyl)-4H-pyrido[1,2-a]pyrimidine-2-carboxamide;
4-oxo-N-({6-[({[(2R)-oxolan-2-yl]methyl}amino)methyl]imidazo[1,2-a]pyridin-2-yl}methyl)-4H-pyrido[1,2-a]pyrimidine-2-carboxamide;
N-[(6-{[(2,2-difluoroethyl)amino]methyl}imidazo[1,2-a]pyridin-2-yl)methyl]-4-oxo-4H-pyrido[1,2-a]pyrimidine-2-carboxamide;
4-oxo-N-{[6-({[(oxolan-3-yl)methyl]amino}methyl)imidazo[1,2-a]pyridin-2-yl]methyl}-4H-pyrido[1,2-a]pyrimidine-2-carboxamide;
N-{[6-({[(1-methylcyclopropyl)methyl]amino}methyl)imidazo[1,2-a]pyridin-2-yl]methyl}-4-oxo-4H-pyrido[1,2-a]pyrimidine-2-carboxamide;
4-oxo-N-[(6-{[(oxolan-3-yl)amino]methyl}imidazo[1,2-a]pyridin-2-yl)methyl]-4H-pyrido[1,2-a]pyrimidine-2-carboxamide;
methyl 3-methyl-2-[({2-[({4-oxo-4H-pyrido[1,2-a]pyrimidin-2-yl}formamido)methyl]imidazo[1,2-a]pyridin-6-yl}methyl)amino]butanoate;
N-[(6-{[(oxan-3-yl)amino]methyl}imidazo[1,2-a]pyridin-2-yl)methyl]-4-oxo-4H-pyrido[1,2-a]pyrimidine-2-carboxamide;
4-oxo-N-{[6-({[(2S)-3,3,3-trifluoro-2-hydroxypropyl]amino}methyl)imidazo[1,2-a]pyridin-2-yl]methyl}-4H-pyrido[1,2-a]pyrimidine-2-carboxamide;
N-({6-[(cyclobutylamino)methyl]imidazo[1,2-a]pyridin-2-yl}methyl)-4-oxo-4H-pyrido[1,2-a]pyrimidine-2-carboxamide;
N-({6-[(tert-butylamino)methyl]imidazo[1,2-a]pyridin-2-yl}methyl)-4-oxo-4H-pyrido[1,2-a]pyrimidine-2-carboxamide;
N-[(6-{[(2-fluoroethyl)amino]methyl}imidazo[1,2-a]pyridin-2-yl)methyl]-4-oxo-4H-pyrido[1,2-a]pyrimidine-2-carboxamide;
N-({6-[(4,4-difluoropiperidin-1-yl)methyl]imidazo[1,2-a]pyridin-2-yl}methyl)-4-oxo-4H-pyrido[1,2-a]pyrimidine-2-carboxamide;
4-oxo-N-({6-[(4-phenylpiperazin-1-yl)methyl]imidazo[1,2-a]pyridin-2-yl}methyl)-4H-pyrido[1,2-a]pyrimidine-2-carboxamide;
4-oxo-N-({6-[(1,2,3,4-tetrahydroisoquinolin-2-yl)methyl]imidazo[1,2-a]pyridin-2-yl}methyl)-4H-pyrido[1,2-a]pyrimidine-2-carboxamide;
N-({6-[(diethylamino)methyl]imidazo[1,2-a]pyridin-2-yl}methyl)-4-oxo-4H-pyrido[1,2-a]pyrimidine-2-carboxamide;
4-oxo-N-({6-[(pyrrolidin-1-yl)methyl]imidazo[1,2-a]pyridin-2-yl}methyl)-4H-pyrido[1,2-a]pyrimidine-2-carboxamide;
4-oxo-N-[(6-{[3-(pyridin-2-yl)azetidin-1-yl]methyl}imidazo[1,2-a]pyridin-2-yl)methyl]-4H-pyrido[1,2-a]pyrimidine-2-carboxamide;
N-[(6-{[(dicyclopropylmethyl)amino]methyl}imidazo[1,2-a]pyridin-2-yl)methyl]-4-oxo-4H-pyrido[1,2-a]pyrimidine-2-carboxamide;
N-[(6-{[(cyclopropylmethyl)(methyl)amino]methyl}imidazo[1,2-a]pyridin-2-yl)methyl]-4-oxo-4H-pyrido[1,2-a]pyrimidine-2-carboxamide;
N-({6-[(4-methylpiperidin-1-yl)methyl]imidazo[1,2-a]pyridin-2-yl}methyl)-4-oxo-4H-pyrido[1,2-a]pyrimidine-2-carboxamide;
4-oxo-N-[(6-{[4-(trifluoromethyl)piperidin-1-yl]methyl}imidazo[1,2-a]pyridin-2-yl)methyl]-4H-pyrido[1,2-a]pyrimidine-2-carboxamide;
N-({6-[(3-methylpiperidin-1-yl)methyl]imidazo[1,2-a]pyridin-2-yl}methyl)-4-oxo-4H-pyrido[1,2-a]pyrimidine-2-carboxamide;
4-oxo-N-[(6-{[({spiro[2.2]pentan-1-yl}methyl)amino]methyl}imidazo[1,2-a]pyridin-2-yl)methyl]-4H-pyrido[1,2-a]pyrimidine-2-carboxamide;
N-({6-[(3,3-dimethylpiperidin-1-yl)methyl]imidazo[1,2-a]pyridin-2-yl}methyl)-4-oxo-4H-pyrido[1,2-a]pyrimidine-2-carboxamide;
4-oxo-N-[(6-{[3-(trifluoromethyl)piperidin-1-yl]methyl}imidazo[1,2-a]pyridin-2-yl)methyl]-4H-pyrido[1,2-a]pyrimidine-2-carboxamide;
N-{[6-({[(5,5-dimethyloxolan-2-yl)methyl]amino}methyl)imidazo[1,2-a]pyridin-2-yl]methyl}-4-oxo-4H-pyrido[1,2-a]pyrimidine-2-carboxamide;
N-({6-[(4-fluoropiperidin-1-yl)methyl]imidazo[1,2-a]pyridin-2-yl}methyl)-4-oxo-4H-pyrido[1,2-a]pyrimidine-2-carboxamide;
N-[(6-{[(3-methoxypropyl)amino]methyl}imidazo[1,2-a]pyridin-2-yl)methyl]-4-oxo-4H-pyrido[1,2-a]pyrimidine-2-carboxamide;
N-[(6-{[(1-methylcyclohexyl)amino]methyl}imidazo[1,2-a]pyridin-2-yl)methyl]-4-oxo-4H-pyrido[1,2-a]pyrimidine-2-carboxamide;
N-{[6-({[(4,4-dimethyloxolan-2-yl)methyl]amino}methyl)imidazo[1,2-a]pyridin-2-yl]methyl}-4-oxo-4H-pyrido[1,2-a]pyrimidine-2-carboxamide;
N-[(6-{[(1-methylcyclopentyl)amino]methyl}imidazo[1,2-a]pyridin-2-yl)methyl]-4-oxo-4H-pyrido[1,2-a]pyrimidine-2-carboxamide;
4-oxo-N-({6-[(propylamino)methyl]imidazo[1,2-a]pyridin-2-yl}methyl)-4H-pyrido[1,2-a]pyrimidine-2-carboxamide;
N-{[6-({[(3,3-dimethyloxolan-2-yl)methyl]amino}methyl)imidazo[1,2-a]pyridin-2-yl]methyl}-4-oxo-4H-pyrido[1,2-a]pyrimidine-2-carboxamide;
N-[(6-{[(2-methylpropyl)amino]methyl}imidazo[1,2-a]pyridin-2-yl)methyl]-4-oxo-4H-pyrido[1,2-a]pyrimidine-2-carboxamide;
N-{[6-({[2-(tert-butoxy)ethyl]amino}methyl)imidazo[1,2-a]pyridin-2-yl]methyl}-4-oxo-4H-pyrido[1,2-a]pyrimidine-2-carboxamide;
N-[(6-{[(4-chlorophenyl)amino]methyl}imidazo[1,2-a]pyridin-2-yl)methyl]-4-oxo-4H-pyrido[1,2-a]pyrimidine-2-carboxamide;
N-{[6-({[2-(oxan-2-yl)ethyl]amino}methyl)imidazo[1,2-a]pyridin-2-yl]methyl}-4-oxo-4H-pyrido[1,2-a]pyrimidine-2-carboxamide;
N-({6-[(4-benzylpiperidin-1-yl)methyl]imidazo[1,2-a]pyridin-2-yl}methyl)-4-oxo-4H-pyrido[1,2-a]pyrimidine-2-carboxamide;
4-oxo-N-({6-[(4-phenoxypiperidin-1-yl)methyl]imidazo[1,2-a]pyridin-2-yl}methyl)-4H-pyrido[1,2-a]pyrimidine-2-carboxamide;
N-{[6-({[(2,2-difluorocyclopropyl)methyl]amino}methyl)imidazo[1,2-a]pyridin-2-yl]methyl}-4-oxo-4H-pyrido[1,2-a]pyrimidine-2-carboxamide;
N-{[6-({[(2,2-dimethylcyclopropyl)methyl]amino}methyl)imidazo[1,2-a]pyridin-2-yl]methyl}-4-oxo-4H-pyrido[1,2-a]pyrimidine-2-carboxamide;
N-{[6-({[(2-methyloxolan-2-yl)methyl]amino}methyl)imidazo[1,2-a]pyridin-2-yl]methyl}-4-oxo-4H-pyrido[1,2-a]pyrimidine-2-carboxamide;
N-({6-[(4-tert-butylpiperidin-1-yl)methyl]imidazo[1,2-a]pyridin-2-yl}methyl)-4-oxo-4H-pyrido[1,2-a]pyrimidine-2-carboxamide;
N-[(6-{[(4-tert-butylcyclohexyl)amino]methyl}imidazo[1,2-a]pyridin-2-yl)methyl]-4-oxo-4H-pyrido[1,2-a]pyrimidine-2-carboxamide;
N-[(6-{[(2-cyclopentylethyl)amino]methyl}imidazo[1,2-a]pyridin-2-yl)methyl]-4-oxo-4H-pyrido[1,2-a]pyrimidine-2-carboxamide;
N-[(6-{[4-(2,2-dimethylpropanoyl)piperazin-1-yl]methyl}imidazo[1,2-a]pyridin-2-yl)methyl]-4-oxo-4H-pyrido[1,2-a]pyrimidine-2-carboxamide;
N-({6-[(4-acetylpiperazin-1-yl)methyl]imidazo[1,2-a]pyridin-2-yl}methyl)-4-oxo-4H-pyrido[1,2-a]pyrimidine-2-carboxamide;
N-{[6-({7-azabicyclo[2.2.1]heptan-7-yl}methyl)imidazo[1,2-a]pyridin-2-yl]methyl}-4-oxo-4H-pyrido[1,2-a]pyrimidine-2-carboxamide;
N-({6-[(4,4-dimethylpiperidin-1-yl)methyl]imidazo[1,2-a]pyridin-2-yl}methyl)-4-oxo-4H-pyrido[1,2-a]pyrimidine-2-carboxamide;
N-({6-[(2,2-dimethylpiperidin-1-yl)methyl]imidazo[1,2-a]pyridin-2-yl}methyl)-4-oxo-4H-pyrido[1,2-a]pyrimidine-2-carboxamide;
4-oxo-N-[(6-{[(2,3,3-trimethylbutan-2-yl)amino]methyl}imidazo[1,2-a]pyridin-2-yl)methyl]-4H-pyrido[1,2-a]pyrimidine-2-carboxamide;
N-[(6-{[(5-fluoropyridin-2-yl)amino]methyl}imidazo[1,2-a]pyridin-2-yl)methyl]-4-oxo-4H-pyrido[1,2-a]pyrimidine-2-carboxamide;
N-({6-[({[1,1′-bi(cyclopropane)]-1-yl}amino)methyl]imidazo[1,2-a]pyridin-2-yl}methyl)-4-oxo-4H-pyrido[1,2-a]pyrimidine-2-carboxamide;
N-{[6-({[(1-fluorocyclopentyl)methyl]amino}methyl)imidazo[1,2-a]pyridin-2-yl]methyl}-4-oxo-4H-pyrido[1,2-a]pyrimidine-2-carboxamide;
N-({6-[(2,6-dimethylmorpholin-4-yl)methyl]imidazo[1,2-a]pyridin-2-yl}methyl)-4-oxo-4H-pyrido[1,2-a]pyrimidine-2-carboxamide;
methyl 1-({2-[({4-oxo-4H-pyrido[1,2-a]pyrimidin-2-yl}formamido)methyl]imidazo[1,2-a]pyridin-6-yl}methyl)piperidine-3-carboxylate;
N-[(6-{[(2-fluoro-2-methylpropyl)amino]methyl}imidazo[1,2-a]pyridin-2-yl)methyl]-4-oxo-4H-pyrido[1,2-a]pyrimidine-2-carboxamide;
N-[(6-{[(1-cyclohexylethyl)amino]methyl}imidazo[1,2-a]pyridin-2-yl)methyl]-4-oxo-4H-pyrido[1,2-a]pyrimidine-2-carboxamide;
N-[(6-{[(2-cyclopropylethyl)(methyl)amino]methyl}imidazo[1,2-a]pyridin-2-yl)methyl]-4-oxo-4H-pyrido[1,2-a]pyrimidine-2-carboxamide;
N-[(6-{[(2,2-dimethylpropyl)(methyl)amino]methyl}imidazo[1,2-a]pyridin-2-yl)methyl]-4-oxo-4H-pyrido[1,2-a]pyrimidine-2-carboxamide;
N-{[6-({[(1-fluorocyclobutyl)methyl]amino}methyl)imidazo[1,2-a]pyridin-2-yl]methyl}-4-oxo-4H-pyrido[1,2-a]pyrimidine-2-carboxamide;
N-({6-[(4-fluoro-4-methylpiperidin-1-yl)methyl]imidazo[1,2-a]pyridin-2-yl}methyl)-4-oxo-4H-pyrido[1,2-a]pyrimidine-2-carboxamide;
N-({6-[(3,3-difluoropiperidin-1-yl)methyl]imidazo[1,2-a]pyridin-2-yl}methyl)-4-oxo-4H-pyrido[1,2-a]pyrimidine-2-carboxamide;
N-{[6-({[(1-hydroxycyclohexyl)methyl]amino}methyl)imidazo[1,2-a]pyridin-2-yl]methyl}-4-oxo-4H-pyrido[1,2-a]pyrimidine-2-carboxamide;
N-[(6-{[(cyclopentylmethyl)amino]methyl}imidazo[1,2-a]pyridin-2-yl)methyl]-4-oxo-4H-pyrido[1,2-a]pyrimidine-2-carboxamide;
N-{[6-({[(3,3-difluorocyclopentyl)methyl]amino}methyl)imidazo[1,2-a]pyridin-2-yl]methyl}-4-oxo-4H-pyrido[1,2-a]pyrimidine-2-carboxamide;
N-[(6-{[(cyclobutylmethyl)amino]methyl}imidazo[1,2-a]pyridin-2-yl)methyl]-4-oxo-4H-pyrido[1,2-a]pyrimidine-2-carboxamide;
N-{[6-({[2-(3,3-difluorocyclobutyl)ethyl]amino}methyl)imidazo[1,2-a]pyridin-2-yl]methyl}-4-oxo-4H-pyrido[1,2-a]pyrimidine-2-carboxamide;
N-{[6-({[(2-fluorocyclobutyl)methyl]amino}methyl)imidazo[1,2-a]pyridin-2-yl]methyl}-4-oxo-4H-pyrido[1,2-a]pyrimidine-2-carboxamide;
N-{[6-({[(2S)-3,3-dimethylbutan-2-yl]amino}methyl)imidazo[1,2-a]pyridin-2-yl]methyl}-4-oxo-4H-pyrido[1,2-a]pyrimidine-2-carboxamide;
N-{[6-({6-azaspiro[2.5]octan-6-yl}methyl)imidazo[1,2-a]pyridin-2-yl]methyl}-4-oxo-4H-pyrido[1,2-a]pyrimidine-2-carboxamide;
4-oxo-N-({6-[({[(1r,3r)-3-fluorocyclobutyl]methyl}amino)methyl]imidazo[1,2-a]pyridin-2-yl}methyl)-4H-pyrido[1,2-a]pyrimidine-2-carboxamide;
4-oxo-N-({6-[(2-phenylethyl)amino]imidazo[1,2-a]pyridin-2-yl}methyl)-4H-pyrido[1,2-a]pyrimidine-2-carboxamide;
N-({6-[2-(benzylamino)ethyl]imidazo[1,2-a]pyridin-2-yl}methyl)-4-oxo-4H-pyrido[1,2-a]pyrimidine-2-carboxamide;
N-({6-[2-(cyclohexylamino)ethyl]imidazo[1,2-a]pyridin-2-yl}methyl)-4-oxo-4H-pyrido[1,2-a]pyrimidine-2-carboxamide;
4-oxo-N-({6-[(phenylformamido)methyl]imidazo[1,2-a]pyridin-2-yl}methyl)-4H-pyrido[1,2-a]pyrimidine-2-carboxamide;
N-({6-[(cyclohexylformamido)methyl]imidazo[1,2-a]pyridin-2-yl}methyl)-4-oxo-4H-pyrido[1,2-a]pyrimidine-2-carboxamide;
4-oxo-N-{[6-({[(piperidin-3-yl)methyl]amino}methyl)imidazo[1,2-a]pyridin-2-yl]methyl}-4H-pyrido[1,2-a]pyrimidine-2-carboxamide;
4-oxo-N-{[6-({[(piperidin-2-yl)methyl]amino}methyl)imidazo[1,2-a]pyridin-2-yl]methyl}-4H-pyrido[1,2-a]pyrimidine-2-carboxamide;
N-[(6-{[(azetidin-3-yl)amino]methyl}imidazo[1,2-a]pyridin-2-yl)methyl]-4-oxo-4H-pyrido[1,2-a]pyrimidine-2-carboxamide;
N-({6-[(cyclohexylamino)methyl]imidazo[1,2-a]pyridin-2-yl}methyl)-4-oxo-4H-chromene-2-carboxamide;
N-[(6-{[(2-cyclopropylethyl)amino]methyl}imidazo[1,2-a]pyridin-2-yl)methyl]-4-oxo-4H-chromene-2-carboxamide;
4-oxo-N-({6-[(piperidin-1-yl)methyl]imidazo[1,2-a]pyridin-2-yl}methyl)-4H-chromene-2-carboxamide;
N-{[6-({[(4-chlorophenyl)methyl]amino}methyl)imidazo[1,2-a]pyridin-2-yl]methyl}-4-oxo-4H-chromene-2-carboxamide;
N-({6-[(benzylamino)methyl]imidazo[1,2-a]pyridin-2-yl}methyl)-4-oxo-4H-chromene-2-carboxamide;
N-{[6-({[(1-methylcyclohexyl)methyl]amino}methyl)imidazo[1,2-a]pyridin-2-yl]methyl}-4-oxo-4H-chromene-2-carboxamide;
N-[(6-{[(2,2-dimethylpropyl)amino]methyl}imidazo[1,2-a]pyridin-2-yl)methyl]-4-oxo-4H-chromene-2-carboxamide;
4-oxo-N-[(7-{[(2-phenylethyl)amino]methyl}imidazo[1,2-a]pyridin-2-yl)methyl]-4H-pyrido[1,2-a]pyrimidine-2-carboxamide;
N-[(7-{[(cyclohexylmethyl)amino]methyl}imidazo[1,2-a]pyridin-2-yl)methyl]-4-oxo-4H-pyrido[1,2-a]pyrimidine-2-carboxamide;
N-[(7-{[(cyclopropylmethyl)amino]methyl}imidazo[1,2-a]pyridin-2-yl)methyl]-4-oxo-4H-pyrido[1,2-a]pyrimidine-2-carboxamide;
N-[(6-{1-[(cyclohexylmethyl)amino]cyclopropyl}imidazo[1,2-a]pyridin-2-yl)methyl]-4-oxo-4H-pyrido[1,2-a]pyrimidine-2-carboxamide;
N-{[6-(2-amino-3-phenylpropyl)imidazo[1,2-a]pyridin-2-yl]methyl}-4-oxo-4H-pyrido[1,2-a]pyrimidine-2-carboxamide;
(cyclohexylmethyl)[(2-{[4-(1H-indazol-4-yl)-1H-1,2,3-triazol-1-yl]methyl}imidazo[1,2-a]pyridin-6-yl)methyl]amine;
N-(cyclohexylmethyl)-2-{[4-(1H-indazol-4-yl)-1H-1,2,3-triazol-1-yl]methyl}imidazo[1,2-a]pyridine-6-carboxamide;
(2,2-dimethylpropyl)[(2-{[4-(1H-indazol-4-yl)-1H-1,2,3-triazol-1-yl]methyl}imidazo[1,2-a]pyridin-6-yl)methyl]amine;
4-[1-({6-[(4,4-dimethylpiperidin-1-yl)methyl]imidazo[1,2-a]pyridin-2-yl}methyl)-1H-1,2,3-triazol-4-yl]-1H-indazole;
[(2-{[4-(6-bromo-1H-indazol-4-yl)-1H-1,2,3-triazol-1-yl]methyl}imidazo[1,2-a]pyridin-6-yl)methyl][(3,3-difluorocyclobutyl)methyl]amine;
[(2-{[4-(6-bromo-1H-indazol-4-yl)-1H-1,2,3-triazol-1-yl]methyl}imidazo[1,2-a]pyridin-6-yl)methyl](2,2-dimethylpropyl)amine;
[(2-{[4-(6-bromo-1H-indazol-4-yl)-1H-1,2,3-triazol-1-yl]methyl}imidazo[1,2-a]pyridin-6-yl)methyl](cyclohexylmethyl)amine;
6-bromo-4-[1-({6-[(4,4-dimethylpiperidin-1-yl)methyl]imidazo[1,2-a]pyridin-2-yl}methyl)-1H-1,2,3-triazol-4-yl]-1H-indazole;
(2-{[4-(6-bromo-1H-indazol-4-yl)-1H-1, 2,3-triazol-1-yl]methyl}imidazo[1,2-a]pyridin-6-yl)methanol;
(2,2-dimethylpropyl)[(2-{[4-(1H-indazol-5-yl)-1H-1,2,3-triazol-1-yl]methyl}imidazo[1,2-a]pyridin-6-yl)methyl]amine;
((cyclohexylmethyl) [1-(2-{[4-(1H-indazol-4-yl)-1H-1,2,3-triazol-1-yl]methyl}imidazo[1,2-a]pyridin-6-yl)ethyl]amine;
(2,2-dimethylpropyl)({2-[(4-{1H-pyrazolo[3,4-c]pyridin-4-yl}-1H-1,2,3-triazol-1-yl)methyl]imidazo[1,2-a]pyridin-6-yl}methyl)amine;
{2-[(4-{1H-pyrazolo[3,4-c]pyridin-4-yl}-1H-1,2,3-triazol-1-yl)methyl]imidazo[1,2-a]pyridin-6-yl}methanol;
N-({6-[(3R,5S)-5-tert-butylmorpholin-3-yl]imidazo[1,2-a]pyridin-2-yl}methyl)-4-oxo-4H-pyrido[1,2-a]pyrimidine-2-carboxamide;
N-({6-[(3S,5R)-5-tert-butylmorpholin-3-yl]imidazo[1,2-a]pyridin-2-yl}methyl)-4-oxo-4H-pyrido[1,2-a]pyrimidine-2-carboxamide;
N-({6-[(3S,5R)-5-cyclohexylmorpholin-3-yl]imidazo[1,2-a]pyridin-2-yl}methyl)-4-oxo-4H-pyrido[1,2-a]pyrimidine-2-carboxamide;
N-({6-[(3S,5R)-5-cyclohexylmorpholin-3-yl]imidazo[1,2-a]pyridin-2-yl}methyl)-1H-indazole-4-carboxamide;
N-({6-[(3S,5R)-5-cyclohexylmorpholin-3-yl]imidazo[1,2-a]pyridin-2-yl}methyl)-4-oxo-4H-chromene-2-carboxamide;
N-({6-[4-(2,2-dimethylpropyl)morpholin-3-yl]imidazo[1,2-a]pyridin-2-yl}methyl)-4-oxo-4H-pyrido[1,2-a]pyrimidine-2-carboxamide;
N-({6-[(3S,5R)-5-methylmorpholin-3-yl]imidazo[1,2-a]pyridin-2-yl}methyl)-4-oxo-4H-pyrido[1,2-a]pyrimidine-2-carboxamide; and
N-{[6-(9-methoxy-2,3,4,5-tetrahydro-1,4-benzoxazepin-3-yl)imidazo[1,2-a]pyridin-2-yl]methyl}-4-oxo-4H-pyrido[1,2-a]pyrimidine-2-carboxamide;
4-[1-[[6-[(cyclobutylmethylamino)methyl]imidazo[1,2-a]pyridin-2-yl]methyl]triazol-4-yl]-1H-indazol-6-ol;
4-[1-[[6-[[(1-hydroxycyclobutyl)methylamino]methyl]imidazo[1,2-a]pyridin-2-yl]methyl]triazol-4-yl]-1H-indazol-6-ol;
1-[[[2-[[4-(5-methoxy-1H-indazol-4-yl)triazol-1-yl]methyl]imidazo[1,2-a]pyridin-6-yl]methylamino]methyl]cyclobutanol;
1-[[[2-[[4-(6-methoxy-1H-indazol-4-yl)triazol-1-yl]methyl]imidazo[1,2-a]pyridin-6-yl]methylamino]methyl]cyclobutanol;
N-(cyclobutylmethyl)-1-[2-[[4-(6-methoxy-1H-indazol-4-yl)triazol-1-yl]methyl]imidazo[1,2-a]pyridin-6-yl]methanamine;
N-(cyclobutylmethyl)-1-[2-[[4-(5-methoxy-1H-indazol-4-yl)triazol-1-yl]methyl]imidazo[1,2-a]pyridin-6-yl]methanamine;
4-[1-[(6-methylimidazo[1,2-a]pyridin-2-yl)methyl]triazol-4-yl]-1H-indazol-3-amine
N-[[6-[[(1-methoxycyclobutyl)methylamino]methyl]imidazo[1,2-a]pyridin-2-yl]methyl]-4-oxo-pyrido[1,2-a]pyrimidine-2-carboxamide;
N-[[6-(1,4-oxazepan-3-yl)imidazo[1,2-a]pyridin-2-yl]methyl]-4-oxo-pyrido[1,2-a]pyrimidine-2-carboxamide;
N-[[6-[[(2-cyano-2-methyl-propyl)amino]methyl]imidazo[1,2-a]pyridin-2-yl]methyl]-4-oxo-pyrido[1,2-a]pyrimidine-2-carboxamide;
N-[[6-[[(3-fluoro-1-bicyclo[1.1.1]pentanyl)methylamino]methyl]imidazo[1,2-a]pyridin-2-yl]methyl]-4-oxo-pyrido[1,2-a]pyrimidine-2-carboxamide;
4-oxo-N-[[6-[(spiro[3.3]heptan-2-ylmethylamino)methyl]imidazo[1,2-a]pyridin-2-yl]methyl]pyrido[1,2-a]pyrimidine-2-carboxamide;
4-oxo-N-[[6-[(spiro[2.3]hexan-5-ylmethylamino)methyl]imidazo[1,2-a]pyridin-2-yl]methyl]pyrido[1,2-a]pyrimidine-2-carboxamide;
N-[[6-[(1-bicyclo[1.1.1]pentanylmethylamino)methyl]imidazo[1,2-a]pyridin-2-yl]methyl]-4-oxo-pyrido[1,2-a]pyrimidine-2-carboxamide;
4-oxo-N-[[6-[(spiro[2.3]hexan-2-ylamino)methyl]imidazo[1,2-a]pyridin-2-yl]methyl]pyrido[1,2-a]pyrimidine-2-carboxamide;
N-[[[6-[(2-methoxy-2-methyl-propyl)amino]methyl]imidazo[1,2-a]pyridin-2-yl]methyl]-4-oxo-pyrido[1,2-a]pyrimidine-2-carboxamide;
N-[[6-[[(1-methylcyclobutyl)methylamino]methyl]imidazo[1,2-a]pyridin-2-yl]methyl]-4-oxo-pyrido[1,2-a]pyrimidine-2-carboxamide;
N-[[6-(butylaminomethyl)imidazo[1,2-a]pyridin-2-yl]methyl]-4-oxo-pyrido[1,2-a]pyrimidine-2-carboxamide;
N-[[6-[[(1-hydroxycyclopentyl)methylamino]methyl]imidazo[1,2-a]pyridin-2-yl]methyl]-4-oxo-pyrido[1,2-a]pyrimidine-2-carboxamide;
N-[[6-[(2-methylbutylamino)methyl]imidazo[1,2-a]pyridin-2-yl]methyl]-4-oxo-pyrido[1,2-a]pyrimidine-2-carboxamide;
N-[[6-[(2-cyclobutylethylamino)methyl]imidazo[1,2-a]pyridin-2-yl]methyl]-4-oxo-pyrido[1,2-a]pyrimidine-2-carboxamide;
N-[[6-[[(2-hydroxy-2-methyl-propyl)amino]methyl]imidazo[1,2-a]pyridin-2-yl]methyl]-4-oxo-pyrido[1,2-a]pyrimidine-2-carboxamide;
N-[[6-[[(1-hydroxycyclobutyl)methylamino]methyl]imidazo[1,2-a]pyridin-2-yl]methyl]-4-oxo-pyrido[1,2-a]pyrimidine-2-carboxamide;
N-[[6-[(2-hydroxybutylamino)methyl]imidazo[1,2-a]pyridin-2-yl]methyl]-4-oxo-pyrido[1,2-a]pyrimidine-2-carboxamide;
N-[[6-[[(3-methylcyclobutyl)methylamino]methyl]imidazo[1,2-a]pyridin-2-yl]methyl]-4-oxo-pyrido[1,2-a]pyrimidine-2-carboxamide;
N-[[6-[[(3,3-dimethylcyclobutyl)methylamino]methyl]imidazo[1,2-a]pyridin-2-yl]methyl]-4-oxo-pyrido[1,2-a]pyrimidine-2-carboxamide;
N-[[6-[(2,2-dimethylbutylamino)methyl]imidazo[1,2-a]pyridin-2-yl]methyl]-4-oxo-pyrido[1,2-a]pyrimidine-2-carboxamide;
N-[[6-[[[(2S)-2-methylbutyl]amino]methyl]imidazo[1,2-a]pyridin-2-yl]methyl]-4-oxo-pyrido[1,2-a]pyrimidine-2-carboxamide;
1-[[[2-[[4-(6-bromo-1H-indazol-4-yl)triazol-1-yl]methyl]imidazo[1,2-a]pyridin-6-yl]methylamino]methyl]cyclohexanol;
1-[[[2-[[4-(6-bromo-1H-indazol-4-yl)triazol-1-yl]methyl]imidazo[1,2-a]pyridin-6-yl]methylamino]methyl]cyclobutanol;
1-[2-[[4-(6-bromo-1H-indazol-4-yl)triazol-1-yl]methyl]imidazo[1,2-a]pyridin-6-yl]-N-(cyclobutylmethyl)methanamine;
4-[1-[[6-[(cyclobutylmethylamino)methyl]imidazo[1,2-a]pyridin-2-yl]methyl]triazol-4-yl]-1H-indazole-6-carboxylic acid;
4-[1-[[6-[(cyclobutylmethylamino)methyl]imidazo[1,2-a]pyridin-2-yl]methyl]triazol-4-yl]-1H-indazole-6-carboxamide;
4-[1-[[6-[(cyclobutylmethylamino)methyl]imidazo[1,2-a]pyridin-2-yl]methyl]triazol-4-yl]-N,N-dimethyl-1H-indazole-6-carboxamide;
[4-[1-[[6-[(cyclobutylmethylamino)methyl]imidazo[1,2-a]pyridin-2-yl]methyl]triazol-4-yl]-1H-indazol-6-yl]-morpholino-methanone;
4-[1-[[6-[(cyclobutylmethylamino)methyl]imidazo[1,2-a]pyridin-2-yl]methyl]triazol-4-yl]-N-(2-hydroxyethyl)-1H-indazole-6-carboxamide;
1-[2-[[4-(6-chloro-1H-indazol-4-yl)triazol-1-yl]methyl]imidazo[1,2-a]pyridin-6-yl]-N-(cyclobutylmethyl)methanamine;
1-[[[2-[[4-(6-chloro-1H-indazol-4-yl)triazol-1-yl]methyl]imidazo[1,2-a]pyridin-6-yl]methylamino]methyl]cyclobutanol;
N-[[2-[[4-(6-chloro-1H-indazol-4-yl)triazol-1-yl]methyl]imidazo[1,2-a]pyridin-6-yl]methyl]-2,2-dimethyl-propan-1-amine;
N-(cyclobutylmethyl)-1-[2-[[4-(7-fluoro-1H-indazol-4-yl)triazol-1-yl]methyl]imidazo[1,2-a]pyridin-6-yl]methanamine;
1-[[[2-[[4-(7-fluoro-1H-indazol-4-yl)triazol-1-yl]methyl]imidazo[1,2-a]pyridin-6-yl]methylamino]methyl]cyclohexanol;
N-(cyclohexylmethyl)-1-[2-[[4-(7-fluoro-1H-indazol-4-yl)triazol-1-yl]methyl]imidazo[1,2-a]pyridin-6-yl]methanamine;
1-[[[2-[[4-(7-fluoro-1H-indazol-4-yl)triazol-1-yl]methyl]imidazo[1,2-a]pyridin-6-yl]methylamino]methyl]cyclobutanol;
1-[[[2-[[4-(7-fluoro-1H-indazol-4-yl)triazol-1-yl]methyl]imidazo[1,2-a]pyridin-6-yl]methylamino]methyl]cyclopentanamine;
N-[[6-[(4,4-dimethyl-1-piperidyl)methyl]imidazo[1,2-a]pyridin-2-yl]methyl]-5-fluoro-4-oxo-chromene-2-carboxamide;
N-(cyclobutylmethyl)-1-[2-[[4-(6-morpholino-1H-indazol-4-yl)triazol-1-yl]methyl]imidazo[1,2-a]pyridin-6-yl]methanamine;
6-[2-(2-aminoethoxy)ethoxy]-N-[[6-[(4,4-dimethyl-1-piperidyl)methyl]imidazo[1,2-a]pyridin-2-yl]methyl]-1H-indazole-4-carboxamide;
N-[[6-[1-(cyclobutylmethylamino)-2-phenyl-ethyl]imidazo[1,2-a]pyridin-2-yl]methyl]-4-oxo-pyrido[1,2-a]pyrimidine-2-carboxamide;
N-[[6-[1-[bis(cyclobutylmethyl)amino]-2-phenyl-ethyl]imidazo[1,2-a]pyridin-2-yl]methyl]-4-oxo-pyrido[1,2-a]pyrimidine-2-carboxamide;
1-[2-[[4-(7-chloro-1H-indazol-4-yl)triazol-1-yl]methyl]imidazo[1,2-a]pyridin-6-yl]-N-(cyclobutylmethyl)methanamine;
1-[[[2-[[4-(7-chloro-1H-indazol-4-yl)triazol-1-yl]methyl]imidazo[1,2-a]pyridin-6-yl]methylamino]methyl]cyclobutanol;
[2-[[4-(6-fluoro-1H-indazol-4-yl)triazol-1-yl]methyl]imidazo[1,2-a]pyridin-6-yl]methanol N-(cyclobutylmethyl)-1-[2-[[4-(6-fluoro-1H-indazol-4-yl)triazol-1-yl]methyl]imidazo[1,2-a]pyridin-6-yl]methanamine;
1-[[[2-[[4-(6-fluoro-1H-indazol-4-yl)triazol-1-yl]methyl]imidazo[1,2-a]pyridin-6-yl]methylamino]methyl]cyclobutanol;
N-(cyclohexylmethyl)-1-[2-[[4-(6-fluoro-1H-indazol-4-yl)triazol-1-yl]methyl]imidazo[1,2-a]pyridin-6-yl]methanamine;
N-[[2-[[4-(6-cyclopropyl-1H-indazol-4-yl)triazol-1-yl]methyl]imidazo[1,2-a]pyridin-6-yl]methyl]-2,2-dimethyl-propan-1-amine;
N-[[6-[(cyclobutylmethylamino)methyl]imidazo[1,2-a]pyridin-2-yl]methyl]-1H-indazole-4-carboxamide;
N-(cyclobutylmethyl)-1-[2-[[4-(1H-pyrazolo[4,3-c]pyridin-4-yl)triazol-1-yl]methyl]imidazo[1,2-a]pyridin-6-yl]methanamine;
N-(cyclohexylmethyl)-1-[2-[[4-(1H-pyrazolo[4,3-c]pyridin-4-yl)triazol-1-yl]methyl]imidazo[1,2-a]pyridin-6-yl]methanamine;
1-[[[2-[[4-(1H-pyrazolo[4,3-c]pyridin-4-yl)triazol-1-yl]methyl]imidazo[1,2-a]pyridin-6-yl]methylamino]methyl]cyclobutanol;
N-(cyclobutylmethyl)-1-[2-[[4-(1H-indazol-4-yl)triazol-1-yl]methyl]imidazo[1,2-a]pyridin-6-yl]methanamine;
1-[[[2-[[4-(1H-indazol-4-yl)triazol-1-yl]methyl]imidazo[1,2-a]pyridin-6-yl]methylamino]methyl]cyclohexanol;
2-[[4-(1H-indazol-4-yl)imidazol-1-yl]methyl]-6-methyl-imidazo[1,2-a]pyridine;
N-[[6-[2-cyano-1-(cyclobutylmethylamino)ethyl]imidazo[1,2-a]pyridin-2-yl]methyl]-4-oxo-pyrido[1,2-a]pyrimidine-2-carboxamide;
N-[[6-(6-methylmorpholin-3-yl)imidazo[1,2-a]pyridin-2-yl]methyl]-4-oxo-pyrido[1,2-a]pyrimidine-2-carboxamide;
N-[[6-(7-bromo-9-methoxy-2,3,4,5-tetrahydro-1,4-benzoxazepin-3-yl)imidazo[1,2-a]pyridin-2-yl]methyl]-4-oxo-pyrido[1,2-a]pyrimidine-2-carboxamide;
4-oxo-N-[(6-piperazin-2-ylimidazo[1,2-a]pyridin-2-yl)methyl]pyrido[1,2-a]pyrimidine-2-carboxamide;
N-[[6-(6-cyclohexyl-4-oxo-2-piperidyl)imidazo[1,2-a]pyridin-2-yl]methyl]-4-oxo-pyrido[1,2-a]pyrimidine-2-carboxamide;
1-[2-(1H-indazol-4-yl) thiazol-5-yl]-1-(6-methylimidazo[1,2-a]pyridin-2-yl)ethanol;
2-[[1-(1H-indazol-4-yl)triazol-4-yl]methyl]imidazo[1,2-a]pyridine;
N-[(3,3-difluorocyclobutyl)methyl]-1-[2-[[1-(1H-indazol-4-yl)triazol-4-yl]methyl]imidazo[1,2-a]pyridin-6-yl]methanamine;
4-[1-[[6-[(cyclobutylmethylamino)methyl]imidazo[1,2-a]pyridin-2-yl]methyl]triazol-4-yl]-1H-indazole-6-carbonitrile;
N-(cyclobutylmethyl)-1-[2-[[4-(1H-indazol-4-yl)imidazol-1-yl]methyl]imidazo[1,2-a]pyridin-6-yl]methanamine;
N-[[6-[[acetyl(cyclobutylmethyl)amino]methyl]imidazo[1,2-a]pyridin-2-yl]methyl]-4-oxo-pyrido[1,2-a]pyrimidine-2-carboxamide;
N-(cyclobutylmethyl)-1-[2-[[3-(1H-indazol-4-yl)-1,2,4-triazol-1-yl]methyl]imidazo[1,2-a]pyridin-6-yl]methanamine;
2-[1-[[6-[(cyclobutylmethylamino)methyl]imidazo[1,2-a]pyridin-2-yl]methyl]triazol-4-yl]pyrido[1,2-a]pyrimidin-4-one;
N-[[6-[(2-bicyclo[2.2.1]hept-5-enylmethylamino)methyl]imidazo[1,2-a]pyridin-2-yl]methyl]-4-oxo-pyrido[1,2-a]pyrimidine-2-carboxamide;
N-[[6-[[[(1R,2R,4S)-norbornan-2-yl]methylamino]methyl]imidazo[1,2-a]pyridin-2-yl]methyl]-4-oxo-pyrido[1,2-a]pyrimidine-2-carboxamide;
N-[[6-[[[(1R,2S,4S)-norbornan-2-yl]methylamino]methyl]imidazo[1,2-a]pyridin-2-yl]methyl]-4-oxo-pyrido[1,2-a]pyrimidine-2-carboxamide;
N-[[6-(2-oxa-5-azabicyclo[2.2.1]heptan-5-ylmethyl)imidazo[1,2-a]pyridin-2-yl]methyl]-4-oxo-pyrido[1,2-a]pyrimidine-2-carboxamide;
N-[[6-(2-azabicyclo[2.2.1]heptan-2-ylmethyl)imidazo[1,2-a]pyridin-2-yl]methyl]-4-oxo-pyrido[1,2-a]pyrimidine-2-carboxamide;
N-[[6-(2-azabicyclo[2.2.2]octan-2-ylmethyl)imidazo[1,2-a]pyridin-2-yl]methyl]-4-oxo-pyrido[1,2-a]pyrimidine-2-carboxamide;
N-[[6-[[(2,2-difluorospiro[3.3]heptan-6-yl)amino]methyl]imidazo[1,2-a]pyridin-2-yl]methyl]-4-oxo-pyrido[1,2-a]pyrimidine-2-carboxamide;
4-oxo-N-[[6-[[[rac-(1S,2S,4S)-7-oxabicyclo[2.2.1]hept-5-en-2-yl]methylamino]methyl]imidazo[1,2-a]pyridin-2-yl]methyl]pyrido[1,2-a]pyrimidine-2-carboxamide;
4-oxo-N-[[6-[[[rac-(1S,2R,4S)-7-oxabicyclo[2.2.1]hept-5-en-2-yl]methylamino]methyl]imidazo[1,2-a]pyridin-2-yl]methyl]pyrido[1,2-a]pyrimidine-2-carboxamide; and
N-[(1-methoxycyclobutyl)methyl]-1-[2-[[4-(6-methoxy-1H-indazol-4-yl)triazol-1-yl]methyl]imidazo[1,2-a]pyridin-6-yl]methanamine.
   (iii) Compounds of Formulae (II), (VI) or (VII)

A further group of METTL3 inhibitors is defined by the formula II shown below:

X—Y—Z (II)
wherein:
X is selected from:

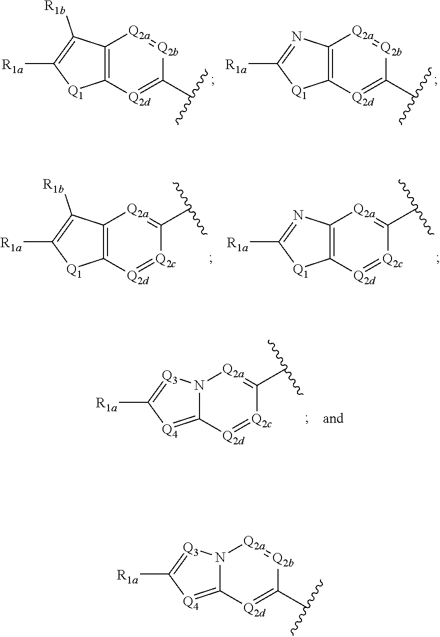

wherein
Q₁ is selected from NH, N—C₁₋₄alkyl, O or S;
Q₂ₐ is selected from N or CR₂ₐ;
Q_{2b} is selected from N or CR_{2b};
Q_{2c} is selected from N or CR_{2c};
Q_{2d} is selected from N or CR_{2d};
Q₃ is selected from N or CR_{1b};
Q₄ is selected from N or CR₁ₓ;
subject to the proviso that no more than 3 of Q₁, Q₂ₐ, Q_{2b}, Q_{2c}, Q_{2d}, Q₃ and Q₄ are nitrogen;
R₁ₐ is selected from:
   (i) C₁₋₄alkyl or C₁₋₄alkoxy, each of which being optionally substituted by halo, cyano, hydroxy, C₃₋₆cycloalkyl, C₁₋₄alkoxy, C₁₋₄haloalkoxy, aryl or heteroaryl; or
   (ii) a group of the formula:

—(CR_{1c}R_{1d})ₚ—NR₁ₑR_{1f};
wherein
   p is an integer selected from 0, 1, 2 or 3
   R_{1c} and R_{1d} are independently selected from:
      (i) hydrogen (including deuterium),
      (ii) C₁₋₆alkyl which is optionally substituted by one more substituents selected from cyano, oxo, hydroxy, C₁₋₄alkoxy, halo, C₁₋₄haloalkoxy, C₃₋₆cycloalkyl, —O—C₃₋₆cycloalkyl, NR_{1ca}R_{1da} or —S(O)₀₋₂R_{1ca}R_{1da}, wherein R_{1ca} and R_{1da} are H or C₁₋₂alkyl; and wherein C₃₋₆cycloalkyl and —O—C₃₋₆cycloalkyl are optionally further substituted with halo, cyano or hydroxy;
      (iii) C₃₋₄cycloalkyl or 3 to 5 membered heterocyclyl, each of which is optionally substituted by C₁₋₄alkyl, C₁₋₄haloalkyl, cyano, hydroxy, C₁₋₂alkoxy, halo, C₁₋₂haloalkoxy, NR_{1ca}R_{1da} or —S(O)₀₋₂R_{1ca}R_{1da}, Wherein R_{1ca} and R_{1da} are H or C₁₋₂alkyl; and;
      (iv) or R_{1c} and R_{1d} are linked together such that, together with the carbon atom to which they are attached, they form a 3- to 6-membered cycloalkyl or heterocyclic ring, or a spirocyclic ring system, each of which is optionally substituted by one or more substituents selected from C₁₋₂alkyl, C₁₋₂haloalkyl, cyano, hydroxy, C₁₋₂alkoxy, halo, C₁₋₂haloalkoxy, NR_{1ca}R_{1da} or —S(O)₀₋₂R_{1ca}R_{1da}, wherein R_{1ca} and R_{1da} are H or C₁₋₂alkyl;
   R₁ₑ and R_{1f} are each independently selected from:
      (i) hydrogen (including deuterium);
      (ii) C₁₋₆alkyl which is optionally substituted by one more substituents selected from cyano, oxo, hydroxy, C₁₋₂alkoxy, halo, C₁₋₂haloalkoxy, NR₁ₑₐR_{1fa} or —S(O)₀₋₂R₁ₑₐR_{1fa}, wherein R₁ₑₐ and R_{1fa} are H or C₁₋₂alkyl;
      (iii) a group with the formula:

—(CR_{1g}R₁ₕ)_{q}-T₁
wherein:
q is 0, 1, 2, 3, 4, 5 or 6;
R_{1g} and R₁ₕ are independently selected from:
a) hydrogen;
b) C₁₋₆alkyl which is optionally substituted by one more substituents selected from cyano, hydroxy, C₁₋₄alkoxy, halo, C₁₋₄haloalkoxy, —O—C₃₋₆cycloalkyl, NR_{1ga}R₁ₕₐ or —S(O)₀₋₂R_{1ga}R₁ₕₐ, wherein R_{1ga} and R₁ₕₐ are H or C₁₋₂alkyl; and wherein —O—C₃₋₆cycloalkyl is optionally substituted with halo, cyano or hydroxy;
c) an aryl-C₁₋₆alkyl, heteroarylC₁₋₆alkyl, C₃₋₆-cycloalkyl or C₃₋₆cycloalkylC₁₋₆alkyl group, each of which is optionally substituted by one or more substituents selected from C₁₋₂alkyl, cyano, C₁₋₂haloalkyl, hydroxy, C₁₋₂alkoxy, halo, C₁₋₂-haloalkoxy, NR_{1ga}R₁ₕₐ or —S(O)₀₋₂R_{1ga}R₁ₕₐ, wherein R_{1ga} and R₁ₕₐ are H or C₁₋₂alkyl; or
d) or R_{1g} and R₁ₕ are optionally linked together such that, together with the carbon atom to which they are attached, they form a 3- to 6-membered cycloalkyl or heterocyclic ring which is optionally substituted by one or more substituents selected from C₁₋₂alkyl, cyano, C₁₋₂haloalkyl, hydroxy, C₁₋₂alkoxy, halo, C₁₋₂haloalkoxy, NR_{1ga}R₁ₕₐ or —S(O)₀₋₂R_{1ga}R₁ₕₐ, wherein R_{1ga} and R₁ₕₐ are H or C₁₋₂alkyl;
and T₁ is selected from hydrogen, cyano, hydroxy, NR₁ₜR₂ₜ or —S(O)₀₋₂R₁ₜR₂ₜ (wherein R₁ₜ and R₂ₜ are H or C₁₋₄alkyl), C₃₋₈cycloalkyl, C₂₋₃alkenyl, C₂₋₃alkynyl, aryl, heterocyclyl, heteroaryl, a spirocyclic carbocyclic or heterocyclic ring system, a bridged C₃₋₈cycloalkyl, a bridged bicyclic C₅₋₁₂cycloalkyl, or a bridged heterocyclic ring system, each of which is optionally substituted by one or more substituents selected from C₁₋₂alkyl, C₁₋₂-haloalkyl, cyano, hydroxy, C₁₋₂alkoxy, halo, C₁₋₂-haloalkoxy, NR₃ₜR₄ₜ or —S(O)₀₋₂R₃ₜR₄ₜ, wherein R₃ₜ and R₄ₜ are H or C₁₋₂alkyl;
(iv) or R₁ₑ and R_{1f} are linked such that, together with the nitrogen atom to which they are attached, they form a mono- or bicyclic-heterocyclic ring, which is optionally substituted by one or more substituents selected from C₁₋₄alkyl, C₁₋₄haloalkyl, cyano, hydroxy, C₁₋₄alkoxy, halo, C₁₋₄haloalkoxy, NR₁ᵢR₁ⱼ or —S(O)₀₋₂R₁ᵢR₁ⱼ, wherein R₁ᵢ and R₁ⱼ are H or C₁₋₄alkyl, and/or the mono- or bicyclic heterocyclic ring formed by R₁ₑ and R_{1f} is optionally spiro-fused to a C₃₋₆cycloalkyl or a heterocyclic ring, which in turn is optionally substituted by one or more substituents selected from C₁₋₄alkyl, C₁₋₄haloalkyl, cyano, hydroxy, C₁₋₄alkoxy, halo, C₁₋₄haloalkoxy, NR₁ᵢR₁ⱼ or —S(O)₀₋₂R₁ᵢR₁ⱼ, wherein R₁ᵢ and R₁ⱼ are H or C₁₋₄alkyl;
   R_{1b} is selected from hydrogen, cyano, halo or C₁₋₃ alkyl;
   R₁ₓ is selected from hydrogen, cyano, halo or C₁₋₃ alkyl;
   R₂ₐ, R_{2b}, R_{2c} and R_{2d} are independently selected from hydrogen, cyano, halo or a group of the formula:

-L₂ₐ-L_{2b}-Q₂

wherein
L₂ₐ is absent or C₁₋₃alkylene optionally substituted by C₁₋₂ alkyl or oxo;
L_{2b} is absent or selected from O, S, SO, SO₂, N(Rₙ), C(O), C(O)O, OC(O), C(O)N(Rₙ), N(Rₙ)C(O), N(Rₙ)C(O)N(Rₒ), S(O)₂N(Rₙ), or N(Rₙ)SO₂, wherein Rₙ and Rₒ are each independently selected from hydrogen or C₁₋₂alkyl; and
Q₂ is hydrogen, cyano, C₁₋₆alkyl, C₃₋₆cycloalkyl, aryl, heterocyclyl or heteroaryl, each of which is optionally substituted by one or more substituents selected from halo, trifluoromethyl, trifluoromethoxy, amino, cyano, hydroxy, amino, carboxy, carbamoyl, sulphamoyl, C₁₋₄alkyl, NRₚR_{q}, ORₚ, C(O)Rₚ, C(O)ORₚ, OC(O)Rₚ, C(O)N(Rₚ)R_{q}, N(Rᵣ)C(O)Rₚ, S(O)_{y}Rₚ (where y is 0, 1 or 2), SO₂N(Rₚ)R_{q}, N(Rᵣ)SO₂Rₚ or (CH₂)₂NRₚR_{q} (where z is 1, 2 or 3), wherein Rₚ and R_{q} are each independently selected from hydrogen or C₁₋₄alkyl;
   Y is selected from:

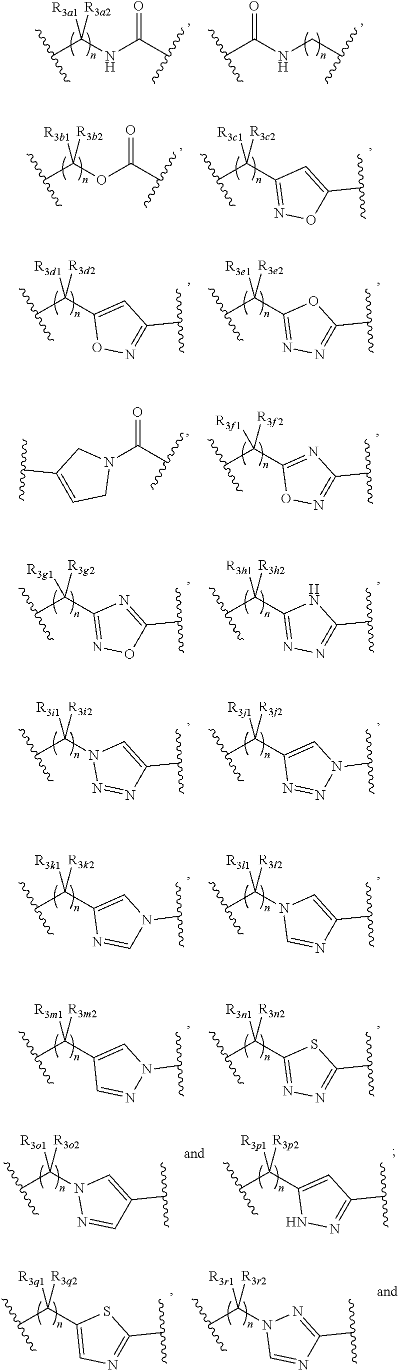

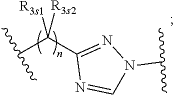

wherein:
R₃ₐ₁, R_{3b1}, R_{3c1}, R_{3d1}, R₃ₑ₁, R_{3f1}, R_{3g1}, R₃ₕ₁, R₃ᵢ₁, R₃ⱼ₁, R₃ₖ₁, R₃ₗ₁, R₃ₘ₁, R₃ₙ₁, R₃ₒ₁, R₃ₚ₁, R_{3q1}, R₃ᵣ₁ and R₃ₛ₁ are independently selected from hydrogen (including deuterium), C₁₋₆alkyl, C₃₋₄ cycloalkyl, hydroxy, and halo; and wherein C₁₋₆alkyl, or C₃₋₄ cycloalkyl is optionally substituted with one or more substituents selected from halo, amino, cyano, and hydroxy;
R₃ₐ₂, R_{3b2}, R_{3c2}, R_{3d2}, R₃ₑ₂, R_{3f2}, R_{3g2}, R₃ₕ₂, R₃ᵢ₂, R₃ⱼ₂, R₃ₖ₂, R₃ₗ₂, R₃ₘ₂, R₃ₙ₂, R₃ₒ₂, R₃ₚ₂, R_{3q2}, R₃ᵣ₂ and R₃ₛ₂ are hydrogen or halo;
with the proviso that R₃ₐ₁, R_{3b1}, R₃ᵢ₁, R₃ₗ₁, R₃ₒ₁, R₃ᵣ₁, R₃ₐ₂, R_{3b2}, R₃ᵢ₂, R₃ₗ₂, R₃ₒ₂ and R₃ₛ₁ cannot be halo when n=1 or when n=2 and the carbon atom to which they are attached is linked to an oxygen or nitrogen atom;
or R₃ₐ₁ and R₃ₐ₂, R_{3b1} and R_{3b2}, R_{3c1} and R_{3c2}, R_{3d1} and R_{3d2}, R₃ₑ₁ and R₃ₑ₂, R_{3f1} and R_{3f2}, R_{3g1} and R_{3g2}, R₃ₕ₁ and R₃ₕ₂, R₃ᵢ₁ and R₃ᵢ₂, R₃ⱼ₁ and R₃ⱼ₂, R₃ₖ₁ and R₃ₖ₂, R₃ₗ₁ and R₃ₗ₂, R₃ₘ₁ and R₃ₘ₂, R₃ₙ₁ and R₃ₙ₂, R₃ₒ₁ and R₃ₒ₂, R₃ₚ₁ and R₃ₚ₂, R_{3q1} and R_{3q2}, or R₃ᵣ₁ and R₃ᵣ₂ or R₃ₛ₁ and R₃ₛ₂ may be linked such that, together with the carbon atom to which they are attached, they form a spiro-fused C₃₋₄cycloalkyl which is optionally substituted with one or more substituents selected from halo, methyl, amino, cyano, and hydroxy;
n is 0, 1 or 2
   Z is selected from one of the following structures:
   i)

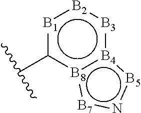

wherein:
B₁ is A₅, wherein A₅ is selected from CR₁₆ and N, wherein R₁₆ is selected from hydrogen, halo, cyano, C₁₋₄ alkyl, C₂₋₄ alkenyl, C₂₋₄ alkynyl, a 5- or 6-membered heteroaryl, C₁₋₄ alkoxy, C₁₋₄haloalkyl, C₁₋₄haloalkoxy, C₃₋₄cycloalkyl, a 3- to 4-membered heterocyclyl and C₃₋₄cycloalkoxy;
B₂ is A₆, wherein A₆ is selected from N or CR₁₇, wherein R₁₇, R_{H2}, R_{H4} and R_{H5} are selected from hydrogen, hydroxy, halo, cyano, C₁₋₅ alkyl, C₁₋₄ haloalkyl, C₁₋₄ alkoxy, C₁₋₄ haloalkoxy, C₂₋₄ alkenyl, C₂₋₄ alkynyl, phenyl, a 5- or 6-membered or heteroaryl, C₃₋₆cycloalkyl, —O—C₃₋₆cycloalkyl, heterocyclyl, —O-heterocyclyl (carbon-linked), —(OCH₂CH₂)ₘ—NR_{q}Rᵣ, —(OCH₂CH₂)ₘ—OCH₃ wherein m is an integer from 1 to 6, NR_{q}Rᵣ, —C(O)—NR_{q}Rᵣ, —C(O)OR_{q},
   wherein R_{q} and Rᵣ are each independently hydrogen, C₁₋₅ alkyl, C₃₋₆cycloalkyl, a 3- to 6-membered carbon-linked heterocyclyl, or R_{q} and Rᵣ are linked together such that, together with the nitrogen atom to which they are attached, they form a 3- to 6-membered heterocyclic ring;
   wherein any C₁₋₅alkyl, C₁₋₄ alkoxy, C₂₋₄alkenyl, C₂₋₄alkynyl, phenyl, 5- or 6-membered or heteroaryl, C₃₋₆cycloalkyl, —O—C₃₋₆cycloalkyl, heterocyclyl or —O-heterocyclyl (carbon-linked) is optionally further substituted by one or more substituents selected from C₁₋₂alkyl, cyano, C₁₋₂haloalkyl, hydroxy, C₁₋₂alkoxy, halo, C₁₋₂haloalkoxy, NR₁ₑₐR_{1fa} or —S(O)₀₋₂R₁ₑₐR_{1fa}, wherein R₁ₑₐ and R_{1fa} are H or C₁₋₂alkyl;
B₃ is N or CR_{Z1}, wherein R_{Z1} is selected from hydrogen, C₁₋₄alkyl, cyano, halo, C₁₋₄haloalkyl, C₁₋₄haloalkoxy, C₁₋₄alkoxy, C₃₋₆cycloalkyl and —O—C₃₋₆cycloalkyl, wherein C₃₋₆cycloalkyl and —O—C₃₋₆cycloalkyl are optionally substituted by one or more of halo, methyl or methoxy;
B₄ is selected from C or N;
B₅ is selected from CR_{zi1b} or NR_{B5N}, wherein:
   R_{Z1b} is selected from hydrogen, C₁₋₄alkyl, cyano, halo, NH₂ and C₁₋₄alkoxy; and
   R_{B5N} is selected from hydrogen or C₁₋₄alkyl;
B₇ is N, NR_{Z2N} or CR_{Z2}, wherein R_{Z2} is selected from hydrogen, C₁₋₄alkyl, cyano, halo,
NH₂ and C₁₋₄alkoxy; and R_{Z2N} is selected from hydrogen or C₁₋₄alkyl;
B₈ is selected from C or N;
with the proviso that no more than four of B₁ to B₈ are N.
   ii)

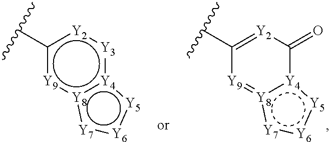

Y₂ is A₇, wherein A₇ is selected from CR₁₈ and N; wherein R₁₈ is selected from hydrogen, halo, cyano, C₁₋₄ alkyl, C₁₋₄ alkoxy, C₁₋₄haloalkyl, C₁₋₄haloalkoxy, C₃₋₄cycloalkyl, a 3- to 4-membered heterocyclyl and C₃₋₄cycloalkoxy;
Y₃ is N or CR_{z1a} wherein R_{Z1a}, is selected from hydrogen, hydroxy, C₁₋₄alkyl, cyano, halo, C₁₋₄ haloalkyl, C₁₋₄haloalkoxy, C₁₋₄alkoxy, C₃₋₆cycloalkyl and —O—C₃₋₆cycloalkyl, wherein C₃₋₆cycloalkyl and —O—C₃₋₆cycloalkyl are optionally substituted by one or more of halo, methyl or methoxy;

### Y₄ is C or N

Y₅ is C—R_{Y5} or NR_{Y5N}, wherein:
R_{Y5} is selected from hydrogen, C₁₋₄alkyl, cyano, halo, NH₂ and C₁₋₄alkoxy;
R_{Y5N} is selected from hydrogen or C₁₋₄alkyl;
   Y₆ is C—R_{Zi2e} or N, wherein R_{Zi2e} is selected from hydrogen, C₁₋₄alkyl, cyano, halo, NH₂ and C₁₋₄alkoxy
   Y₇ is O, S, CR_{Z2a} or N, wherein R_{Z2a} is selected from hydrogen, C₁₋₄alkyl, cyano, halo, NH₂ and C₁₋₄alkoxy;

### Y₈ Is C or N;

Y₉ is CR_{Z3a} or N; wherein
R_{Z3a} is selected from hydrogen, C₁₋₄alkyl, cyano, halo, NH₂ and C₁₋₄alkoxy; with the proviso that no more than four of Y₁ to Y₈ are N.
(iii)

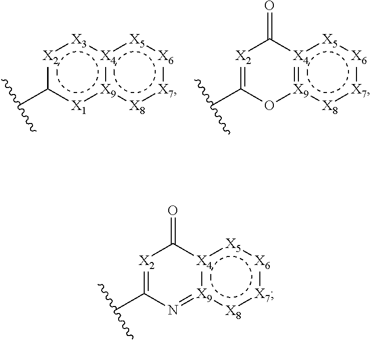

X₁ is N or C—R_{Z9}, wherein R_{Z9} is selected from hydrogen, halo, cyano, C₁₋₄ alkyl, C₁₋₄ haloalkyl, C₁₋₄alkoxy, C₁₋₄ haloalkoxy;
X₂ is selected from N or CR₄ wherein:
   R₄ is selected from hydrogen, halo, cyano, C1-4 alkyl, C1-4 haloalkyl, C1-4alkoxy, C1-4 haloalkoxy (e.g. hydrogen, halo, cyano and methyl);
X₃ is N;
X₄ is N or C;
X₅ is selected from N, CR₅ and CRx₅ₐR_{X5b} wherein:
   R₅ is selected from hydrogen, halo, cyano, C₁₋₄ alkyl, C₁₋₄ haloalkyl, C₁₋₄alkoxy, C₁₋₄ haloalkoxy (e.g. hydrogen, halo, cyano and methyl);
   Rx₅ₐ and R_{X5b} are independently selected from hydrogen, halo, cyano, C₁₋₄alkyl, C₁₋₄ haloalkyl, C₁₋₄alkoxy, C₁₋₄ haloalkoxy (e.g. hydrogen, halo, cyano and methyl);
either:
   X₆ is A₁ and X₇ is A₂; or
   X₆ is A₈ and X₇ is A₉ or A₁₁, wherein:
   A₁ is selected from CR₁₂ and N; wherein
      R₁₂ is selected from selected from hydrogen, halo, cyano, C₁₋₄ alkyl, C₁₋₄ haloalkyl, C₁₋₄alkoxy and C₁₋₄ haloalkoxy (e.g. hydrogen, halo, cyano and C₁₋₄ alkyl);
   A₂ is selected from CR₁₃ and N, wherein
      R₁₃ selected from hydrogen, halo, cyano, C₁₋₄ alkyl, C₁₋₄ haloalkyl, C₁₋₄alkoxy, C₁₋₄ haloalkoxy (e.g. hydrogen, halo, cyano, methoxy and methyl);
   A₈ is selected from CR₁₉R₂₀ and NR₂₁; wherein
      R₁₉ and R₂₀ are independently selected from hydrogen, halo, cyano, C₁₋₄ alkyl, C₁₋₄ haloalkyl, C₁₋₄alkoxy, C₁₋₄ haloalkoxy (e.g. hydrogen, halo, cyano and C₁₋₄ alkyl);
      R₂₁ is hydrogen or C₁₋₄alkyl.
   A₉ is selected from CR₂₂R₂₃ and NR₂₄;
      wherein R₂₂ and R₂₃ are independently selected from selected from hydrogen, halo, cyano, C₁₋₄ alkyl, C₁₋₄ haloalkyl, C₁₋₄alkoxy, C₁₋₄ haloalkoxy (e.g. hydrogen, halo, cyano and methyl);
      R₂₄ is selected from hydrogen or C₁₋₄alkyl
   A₁₁ is selected from CR₂₈R₂₉ and NR₃₀;
      R₂₈ and R₂₉ are selected from hydrogen, halo, methoxy and methyl;
      R₃₀ is selected from hydrogen or C₁₋₄alkyl.
X₈ is selected from CR₆, N or CR_{X6a}R_{X6b};
   wherein R₆ is selected from hydrogen, halo, cyano, C₁₋₄ alkyl, C₁₋₄ haloalkyl, C₁₋₄alkoxy and C₁₋₄ haloalkoxy;
   R_{X6a} and R_{X6b} are each independently selected from hydrogen, halo, cyano, C₁₋₄ alkyl, C₁₋₄ haloalkyl, C₁₋₄alkoxy and C₁₋₄ haloalkoxy;
X₉ is N or C;
with the proviso that no more than four of X₂ to X₉ are N.
   (iv)

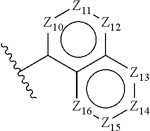

Z₁₀ is N or C—R_{Z10}, wherein R_{Z10} is selected from hydrogen, halo, cyano, C₁₋₄ alkyl, C₁₋₄ haloalkyl, C₁₋₄alkoxy, C₁₋₄ haloalkoxy;
Z₁₁ is N or C—R_{Z11}, wherein R_{Z11} is selected from hydrogen, halo, cyano, C₁₋₄ alkyl, C₁₋₄ haloalkyl, C₁₋₄alkoxy, C₁₋₄ haloalkoxy;
Z₁₂ is N or C—R_{Z12}, wherein R_{Z12} is selected from hydrogen, halo, cyano, C₁₋₄ alkyl, C₁₋₄ haloalkyl, C₁₋₄alkoxy, C₁₋₄ haloalkoxy;
Z₁₃ is N or C—R_{Z13}, wherein R_{Z13} is selected from hydrogen, halo, cyano, C₁₋₄ alkyl, C₁₋₄ haloalkyl, C₁₋₄alkoxy, C₁₋₄ haloalkoxy;
Z₁₄ is N or C—R_{Z14}, wherein R_{Z14} is selected from hydrogen, halo, cyano, C₁₋₄ alkyl, C₁₋₄ haloalkyl, C₁₋₄alkoxy, C₁₋₄ haloalkoxy;
Z₁₅ is N or C—R_{Z15}, wherein R_{Z15} is selected from hydrogen, halo, cyano, C₁₋₄ alkyl, C₁₋₄ haloalkyl, C₁₋₄alkoxy, C₁₋₄ haloalkoxy;
Z₁₆ is N or C—R_{Z16}, wherein R_{Z16} is selected from hydrogen, halo, cyano, C₁₋₄ alkyl, C₁₋₄ haloalkyl, C₁₋₄alkoxy, C₁₋₄ haloalkoxy;
with the proviso that no more than three of Z₁₀ to Z₁₆ are N;
(v)

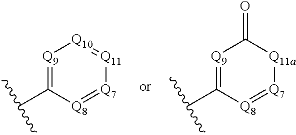

Q₇ is CR₇ or N;
Q₈ is CR₈ or N;
Q₉ is CR₉ or N;
Q₁₀ is CR₁₀ or N;
Q₁₁ is CR₁₁ or N;
Q₁₁ₐ is NR_{11N} or CR₁₁ₐR_{11b};
   wherein R₇, R₈, R₉, R₁₀, R₁₁, R₁₁ₐ and R_{11b} are each independently selected from hydrogen, NH₂, halo, cyano, C₁₋₄ alkoxy, C₁₋₄ haloalkoxy, C₁₋₆ alkyl, —CH₂OCH₃, —CH₂SO₂CH₃, —SO₂CH₃, —NHC(O)CH₃ and —C(O)NRᵥ₁Rᵥ₂, wherein Rᵥ₁ and Rᵥ₂ are independently selected from hydrogen and methyl and; and R_{11N} is selected from hydrogen, NH₂, halo, cyano, and C₁₋₆ alkyl;
   or
R₉ and R₁₀ may be linked together such that, together to the atoms to which they are attached, they form a fused 5- or 6-membered saturated or unsaturated ring system, or R₁₀ and R₁₁ may be linked together such that, together to the atoms to which they are attached, they form a fused 5- or 6-membered saturated or unsaturated ring system, wherein either of the fused 5- or 6-membered saturated or unsaturated ring system may be optionally substituted by one or more substituents selected from C₁₋₂alkyl, cyano, C₁₋₂haloalkyl, hydroxy, C₁₋₂alkoxy, halo, C₁₋₂haloalkoxy, NR₁ᵢₐR₁ⱼₐ or —S(O)₀₋₂R₁ᵢₐR₁ⱼₐ, wherein R₁ᵢₐ and R₁ⱼₐ are H or C₁₋₂alky;
with the proviso that no more than three of Q₇ to Q₁₁ are N.

In a particular group of compounds of formula II above, the compounds are of the formula:

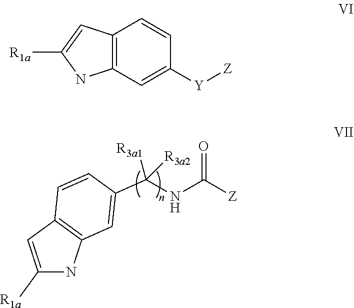

wherein R₁ₐ, Y, Z, n, R₃ₐ₁ and R₃ₐ₂ each have any one of the meanings defined above; or a pharmaceutically acceptable salt thereof.

Suitably, n is 1 and R₃ₐ₁ and R₃ₐ₂ are hydrogen.

Suitably, Ria is a group of the formula:

—(CR_{1c}R_{1d})ₚ—NR₁ₑR_{1f};
wherein
p is 1;
R_{1c} and R_{1d} are independently selected from hydrogen (including deuterium) or C₁₋₂alkyl;
R₁ₑ is selected from hydrogen (including deuterium) or C₁₋₂alkyl; and
R_{1f} is a group with the formula:

—(CR_{1g}R₁ₕ)_{q}-T₁
wherein:
q is 1;
R_{1g} and R₁ₕ are independently selected from hydrogen (including deuterium) or C₁₋₂alkyl;
and T₁ is selected from C₃₋₄cycloalkyl, heterocyclyl, a spirocyclic carbocyclic or heterocyclic ring system, a bridged C₃₋₈cycloalkyl, a bridged bicyclic C₅₋₁₂cycloalkyl, or a bridged heterocyclic ring system, each of which is optionally substituted by one or more substituents selected from C₁₋₂alkyl, C₁₋₂haloalkyl, cyano, hydroxy, C₁₋₂alkoxy, halo, C₁₋₂haloalkoxy or C₃₋₆cycloalkyl,
wherein any alkyl or alkoxy is optionally further substituted by one or more substituents selected from cyano, hydroxy or halo.

Most suitably, R₁ₐ is selected from:

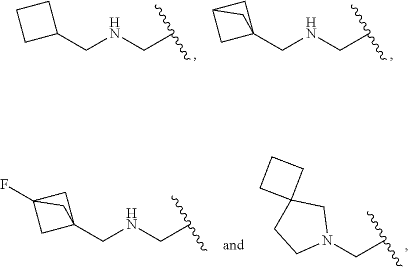

### Suitably Y is:

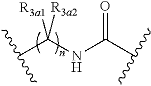

wherein n is 1 and R₃ₐ₁ and R₃ₐ₂ are hydrogen.

### Suitably, Z is:

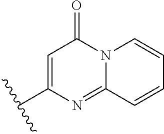 [SMILES: *c1cc(=O)n2ccccc2n1]

### (iv) Specific Compounds of Formula II—List 2

Particular compounds of formula II above include any of the following, or a pharmaceutically acceptable salt thereof:

### List 2

N-({2-[(4,4-dimethylpiperidin-1-yl)methyl]-1H-indol-6-yl}methyl)-4-oxo-4H-pyrido[1,2-a]pyrimidine-2-carboxamide
N-[(2-{[(cyclobutylmethyl)amino]methyl}-1H-indol-6-yl)methyl]-4-oxo-4H-pyrido[1,2-a]pyrimidine-2-carboxamide
N-[[2-[[(3,3-difluorocyclobutyl)methylamino]methyl]-1H-indol-6-yl]methyl]-4-oxo-pyrido[1,2-a]pyrimidine-2-carboxamide
N-[[2-[[(1-hydroxycyclobutyl)methylamino]methyl]-1H-indol-6-yl]methyl]-4-oxo-pyrido[1,2-a]pyrimidine-2-carboxamide
N-[[2-[[(1-fluorocyclobutyl)methylamino]methyl]-1H-indol-6-yl]methyl]-4-oxo-pyrido[1,2-a]pyrimidine-2-carboxamide
N-[[2-[[(1-methylcyclopropyl)methylamino]methyl]-1H-indol-6-yl]methyl]-4-oxo-pyrido[1,2-a]pyrimidine-2-carboxamide.
N-[[2-(2-azabicyclo[2.1.1]hexan-2-ylmethyl)-1H-indol-6-yl]methyl]-4-oxo-pyrido[1,2-a]pyrimidine-2-carboxamide
N-[[2-(3-azabicyclo[3.1.1]heptanean-3-ylmethyl)-1H-indol-6-yl]methyl]-4-oxo-pyrido[1,2-a]pyrimidine-2-carboxamide
N-[[2-[[2-(hydroxymethyl)pyrrolidin-1-yl]methyl]-1H-indol-6-yl]methyl]-4-oxo-pyrido[1,2-a]pyrimidine-2-carboxamide
N-[[2-(morpholinomethyl)-1H-indol-6-yl]methyl]-4-oxo-pyrido[1,2-a]pyrimidine-2-carboxamide
N-[[2-[(1-adamantylamino)methyl]-1H-indol-6-yl]methyl]-4-oxo-pyrido[1,2-a]pyrimidine-2-carboxamide
4-oxo-N-[[2-(1-piperidylmethyl)-1H-indol-6-yl]methyl]pyrido[1,2-a]pyrimidine-2-carboxamide
N-[[2-[(4-fluoro-1-piperidyl)methyl]-1H-indol-6-yl]methyl]-4-oxo-pyrido[1,2-a]pyrimidine-2-carboxamide
4-oxo-N-[[2-[[[rac-(1S,2S,4S)-7-oxabicyclo[2.2.1]heptane-5-en-2-yl]methylamino]methyl]-1H-indol-6-yl]methyl]pyrido[1,2-a]pyrimidine-2-carboxamide
N-[[2-[[(1-hydroxycyclopentyl)methylamino]methyl]-1H-indol-6-yl]methyl]-4-oxo-pyrido[1,2-a]pyrimidine-2-carboxamide
4-oxo-N-[[2-[[[rac-(1S,2R,4S)-7-oxabicyclo[2.2.1]heptane-5-en-2-yl]methylamino]methyl]-1H-indol-6-yl]methyl]pyrido[1,2-a]pyrimidine-2-carboxamide
N-[[2-[(1-bicyclo[1.1.1]pentanylamino)methyl]-1H-indol-6-yl]methyl]-4-oxo-pyrido[1,2-a]pyrimidine-2-carboxamide
N-[[2-[(cyclobutylamino)methyl]-1H-indol-6-yl]methyl]-4-oxo-pyrido[1,2-a]pyrimidine-2-carboxamide
N-({2-[({bicyclo[2.2.1]heptanean-2-yl}amino)methyl]-1H-indol-6-yl}methyl)-4-oxo-4H-pyrido[1,2-a]pyrimidine-2-carboxamide
N-[[2-[(cyclopropylamino)methyl]-1H-indol-6-yl]methyl]-4-oxo-pyrido[1,2-a]pyrimidine-2-carboxamide
N-[[2-(2-azabicyclo[2.2.2]octan-2-ylmethyl)-1H-indol-6-yl]methyl]-4-oxo-pyrido[1,2-a]pyrimidine-2-carboxamide
N-[[2-[[(2,2-difluorocyclopropyl)methylamino]methyl]-1H-indol-6-yl]methyl]-4-oxo-pyrido[1,2-a]pyrimidine-2-carboxamide
N-[(2-{[({3-fluorobicyclo[1.1.1]pentan-1-yl}methyl)amino]methyl}-1H-indol-6-yl)methyl]-4-oxo-4H-pyrido[1,2-a]pyrimidine-2-carboxamide
N-[[2-[(cyclohexylmethylamino)methyl]-1H-indol-6-yl]methyl]-4-oxo-pyrido[1,2-a]pyrimidine-2-carboxamide
N-[[2-[[(1-hydroxycyclohexyl)methylamino]methyl]-1H-indol-6-yl]methyl]-4-oxo-pyrido[1,2-a]pyrimidine-2-carboxamide
N-[[2-[(cyclopentylamino)methyl]-1H-indol-6-yl]methyl]-4-oxo-pyrido[1,2-a]pyrimidine-2-carboxamide
N-[[2-[(cyclopentylmethylamino)methyl]-1H-indol-6-yl]methyl]-4-oxo-pyrido[1,2-a]pyrimidine-2-carboxamide
N-[[2-[[(1-methoxycyclobutyl)methylamino]methyl]-1H-indol-6-yl]methyl]-4-oxo-pyrido[1,2-a]pyrimidine-2-carboxamide
N-[[2-[(isobutylamino)methyl]-1H-indol-6-yl]methyl]-4-oxo-pyrido[1,2-a]pyrimidine-2-carboxamide
N-[[2-[(cyclohexylamino)methyl]-1H-indol-6-yl]methyl]-4-oxo-pyrido[1,2-a]pyrimidine-2-carboxamide
N-[[2-[(cyclopropylmethylamino)methyl]-1H-indol-6-yl]methyl]-4-oxo-pyrido[1,2-a]pyrimidine-2-carboxamide
4-oxo-N-[[2-[(prop-2-ynylamino)methyl]-1H-indol-6-yl]methyl]pyrido[1,2-a]pyrimidine-2-carboxamide
N-[[2-[(oxetan-2-ylmethylamino)methyl]-1H-indol-6-yl]methyl]-4-oxo-pyrido[1,2-a]pyrimidine-2-carboxamide
N-[[2-[(2,2-dimethylpropylamino)methyl]-1H-indol-6-yl]methyl]-4-oxo-pyrido[1,2-a]pyrimidine-2-carboxamide
N-[[2-[(1-bicyclo[1.1.1]pentanylmethylamino)methyl]-1H-indol-6-yl]methyl]-4-oxo-pyrido[1,2-a]pyrimidine-2-carboxamide
N-{[2-({[(1S,2S)-2-hydroxycyclopentyl]amino}methyl)-1H-indol-6-yl]methyl}-4-oxo-4H-pyrido[1,2-a]pyrimidine-2-carboxamide
N-{[2-({[(1R,2R)-2-hydroxycyclopentyl]amino}methyl)-1H-indol-6-yl]methyl}-4-oxo-4H-pyrido[1,2-a]pyrimidine-2-carboxamide
N-{[2-({[(1S,2R)-2-hydroxycyclopentyl]amino}methyl)-1H-indol-6-yl]methyl}-4-oxo-4H-pyrido[1,2-a]pyrimidine-2-carboxamide
N-{[2-({[(1R,2S)-2-hydroxycyclopentyl]amino}methyl)-1H-indol-6-yl]methyl}-4-oxo-4H-pyrido[1,2-a]pyrimidine-2-carboxamide
N-[[2-[(cyclopropylmethylamino)methyl]-5-fluoro-1H-indol-6-yl]methyl]-4-oxo-pyrido[1,2-a]pyrimidine-2-carboxamide
N-[[2-[(cyclobutylmethylamino)methyl]-5-fluoro-1H-indol-6-yl]methyl]-4-oxo-pyrido[1,2-a]pyrimidine-2-carboxamide
4-oxo-N-[[2-[(2,2,2-trifluoroethylamino)methyl]-1H-indol-6-yl]methyl]pyrido[1,2-a]pyrimidine-2-carboxamide
N-[(2-{[N-(cyclobutylmethyl)acetamido]methyl}-1H-indol-6-yl)methyl]-4-oxo-4H-pyrido[1,2-a]pyrimidine-2-carboxamide
4-oxo-N-{[2-(piperidin-2-yl)-1H-indol-6-yl]methyl}-4H-pyrido[1,2-a]pyrimidine-2-carboxamide
N-[(2-{[(cyclobutylmethyl)amino]methyl}-3-fluoro-1H-indol-6-yl)methyl]-4-oxo-4H-pyrido[1,2-a]pyrimidine-2-carboxamide
N-[(2-{[(cyclobutylmethyl)amino]methyl}-1-methyl-1H-indol-6-yl)methyl]-4-oxo-4H-pyrido[1,2-a]pyrimidine-2-carboxamide
N-[[2-[(Cyclobutylmethylamino)-dideuterio-methyl]-1H-indol-6-yl]methyl]-4-oxo-pyrido[1,2-a]pyrimidine-2-carboxamide
N-[[2-[(cyclobutylmethylamino)methyl]-1H-indol-6-yl]methyl]-1H-indazole-4-carboxamide
N-[[2-[(cyclobutylmethylamino)methyl]-1H-pyrrolo[3,2-b]pyridin-6-yl]methyl]-4-oxo-pyrido[1,2-a]pyrimidine-2-carboxamide
N-(1H-indol-6-ylmethyl)-4-oxo-pyrido[1,2-a]pyrimidine-2-carboxamide
N-(1H-indol-2-ylmethyl)-4-oxo-pyrido[1,2-a]pyrimidine-2-carboxamide
N-(indolizin-2-ylmethyl)-4-oxo-pyrido[1,2-a]pyrimidine-2-carboxamide
N-[(6-{[4-(1H-indazol-4-yl)-1H-1,2,3-triazol-1-yl]methyl}-1H-indol-2-yl)methyl]cyclopropanamine
(1R,2S)-2-[[6-[[4-(1H-indazol-4-yl)triazol-1-yl]methyl]-1H-indol-2-yl]methylamino]cyclopentanol
N-[[6-[[4-(1H-indazol-4-yl)triazol-1-yl]methyl]-1H-indol-2-yl]methyl]cyclopentanamine
N-(cyclopropylmethyl)-1-[6-[[4-(1H-indazol-4-yl)triazol-1-yl]methyl]-1H-indol-2-yl]methanamine
1-[[[6-[[4-(1H-indazol-4-yl)triazol-1-yl]methyl]-1H-indol-2-yl]methylamino]methyl]cyclobutanol
N-(cyclobutylmethyl)-1-[6-[[4-(1H-indazol-4-yl)triazol-1-yl]methyl]-1H-indol-2-yl]methanamine
N-(cyclobutylmethyl)-1-[6-[[4-(1H-indazol-4-yl)triazol-1-yl]methyl]-1H-pyrrolo[3,2-b]pyridin-2-yl]methanamine
N-(cyclobutylmethyl)-1-[6-[[4-(1H-indazol-4-yl)triazol-1-yl]methyl]-1H-pyrrolo[3,2-c]pyridin-2-yl]methanamine
N-(cyclobutylmethyl)-1-[6-[[4-(1H-indazol-4-yl)triazol-1-yl]methyl]-1H-pyrrolo[3,2-c]pyridin-2-yl]methanamine
2-[1-[[2-[(cyclobutylmethylamino)methyl]-1H-indol-6-yl]methyl]triazol-4-yl]pyrido[1,2-a]pyrimidin-4-one
N-(cyclobutylmethyl)-1-[6-[[4-(6-methoxyimidazo[1,5-a]pyridin-8-yl)triazol-1-yl]methyl]-1H-indol-2-yl]methanamine
N-(cyclobutylmethyl)-1-[6-[[4-(1H-indazol-4-yl)imidazol-1-yl]methyl]-1H-indol-2-yl]methanamine
N-(cyclobutylmethyl)-1-[6-[[3-(1H-indazol-4-yl)-1,2,4-oxadiazol-5-yl]methyl]-1H-indol-2-yl]methanamine
N-[[2-(2-azaspiro[3.3]heptanean-2-ylmethyl)-1H-indol-6-yl]methyl]-4-oxo-pyrido[1,2-a]pyrimidine-2-carboxamide
N-[[2-[(benzylamino)methyl]-1H-indol-6-yl]methyl]-4-oxo-pyrido[1,2-a]pyrimidine-2-carboxamide
N-[[2-(3-azabicyclo[3.1.0]hexan-3-ylmethyl)-1H-indol-6-yl]methyl]-4-oxo-pyrido[1,2-a]pyrimidine-2-carboxamide
N-[[2-[[cyclobutylmethyl(methyl)amino]methyl]-1H-indol-6-yl]methyl]-4-oxo-pyrido[1,2-a]pyrimidine-2-carboxamide
N-[[2-[(cyclobutylmethylamino)methyl]-1H-pyrrolo[2,3-b]pyridin-6-yl]methyl]-4-oxo-pyrido[1,2-a]pyrimidine-2-carboxamide
N-[(2-{[(cyclobutylmethyl)amino]methyl}-1H-1,3-benzodiazol-6-yl)methyl]-4-oxo-4H-pyrido[1,2-a]pyrimidine-2-carboxamide
N-[(2-{[(but-2-yn-1-yl)amino]methyl}-1H-indol-6-yl)methyl]-4-oxo-4H-pyrido[1,2-a]pyrimidine-2-carboxamide
N-[(2-{[(3-cyclopropylprop-2-yn-1-yl)amino]methyl}-1H-indol-6-yl)methyl]-4-oxo-4H-pyrido[1,2-a]pyrimidine-2-carboxamide
N-({2-[({bicyclo[3.1.0]hexan-6-yl}amino)methyl]-1H-indol-6-yl}methyl)-4-oxo-4H-pyrido[1,2-a]pyrimidine-2-carboxamide
N-[(2-{[({bicyclo[2.1.1]hexan-1-yl}methyl)amino]methyl}-1H-indol-6-yl)methyl]-4-oxo-4H-pyrido[1,2-a]pyrimidine-2-carboxamide
N-[(2-{[({3-methylbicyclo[1.1.1]pentan-1-yl}methyl)amino]methyl}-1H-indol-6-yl)methyl]-4-oxo-4H-pyrido[1,2-a]pyrimidine-2-carboxamide
N-({2-[(3-methylazetidin-1-yl)methyl]-1H-indol-6-yl}methyl)-4-oxo-4H-pyrido[1,2-a]pyrimidine-2-carboxamide
N-({2-[(3-fluoroazetidin-1-yl)methyl]-1H-indol-6-yl}methyl)-4-oxo-4H-pyrido[1,2-a]pyrimidine-2-carboxamide
N-({2-[(azetidin-1-yl)methyl]-1H-indol-6-yl}methyl)-4-oxo-4H-pyrido[1,2-a]pyrimidine-2-carboxamide
N-{[2-({2-azaspiro[3.4]octan-2-yl}methyl)-1H-indol-6-yl]methyl}-4-oxo-4H-pyrido[1,2-a]pyrimidine-2-carboxamide
N-({2-[(3-hydroxyazetidin-1-yl)methyl]-1H-indol-6-yl}methyl)-4-oxo-4H-pyrido[1,2-a]pyrimidine-2-carboxamide
N-({2-[(3,3-dimethylazetidin-1-yl)methyl]-1H-indol-6-yl}methyl)-4-oxo-4H-pyrido[1,2-a]pyrimidine-2-carboxamide
4-oxo-N-[(2-{[3-(2,2,2-trifluoroethoxy)azetidin-1-yl]methyl}-1H-indol-6-yl)methyl]-4H-pyrido[1,2-a]pyrimidine-2-carboxamide
N-[(2-{[3-(difluoromethyl) azetidin-1-yl]methyl}-1H-indol-6-yl)methyl]-4-oxo-4H-pyrido[1,2-a]pyrimidine-2-carboxamide
N-({2-[(3-methoxyazetidin-1-yl)methyl]-1H-indol-6-yl}methyl)-4-oxo-4H-pyrido[1,2-a]pyrimidine-2-carboxamide
N-[(2-{[3-(tert-butoxy)azetidin-1-yl]methyl}-1H-indol-6-yl)methyl]-4-oxo-4H-pyrido[1,2-a]pyrimidine-2-carboxamide
4-oxo-N-[(2-{[3-(trifluoromethyl)azetidin-1-yl]methyl}-1H-indol-6-yl)methyl]-4H-pyrido[1,2-a]pyrimidine-2-carboxamide
N-({2-[(3-ethoxyazetidin-1-yl)methyl]-1H-indol-6-yl}methyl)-4-oxo-4H-pyrido[1,2-a]pyrimidine-2-carboxamide
N-{[2-({2-azaspiro[3.5]nonan-2-yl}methyl)-1H-indol-6-yl]methyl}-4-oxo-4H-pyrido[1,2-a]pyrimidine-2-carboxamide
N-({2-[(2-methylazetidin-1-yl)methyl]-1H-indol-6-yl}methyl)-4-oxo-4H-pyrido[1,2-a]pyrimidine-2-carboxamide
N-({2-[(3,3-dimethylpyrrolidin-1-yl)methyl]-1H-indol-6-yl}methyl)-4-oxo-4H-pyrido[1,2-a]pyrimidine-2-carboxamide
N-{[2-({6-fluoro-2-azaspiro[3.3]heptanean-2-yl}methyl)-1H-indol-6-yl]methyl}-4-oxo-4H-pyrido[1,2-a]pyrimidine-2-carboxamide
N-{[2-({6,6-difluoro-2-azaspiro[3.3]heptanean-2-yl}methyl)-1H-indol-6-yl]methyl}-4-oxo-4H-pyrido[1,2-a]pyrimidine-2-carboxamide
N-({2-[(3-cyclobutylazetidin-1-yl)methyl]-1H-indol-6-yl}methyl)-4-oxo-4H-pyrido[1,2-a]pyrimidine-2-carboxamide
N-({2-[(3-cyclopropylazetidin-1-yl)methyl]-1H-indol-6-yl}methyl)-4-oxo-4H-pyrido[1,2-a]pyrimidine-2-carboxamide
N-({2-[(3-tert-butylazetidin-1-yl)methyl]-1H-indol-6-yl}methyl)-4-oxo-4H-pyrido[1,2-a]pyrimidine-2-carboxamide
N-[(2-{[(1-tert-butylcyclopropyl)amino]methyl}-1H-indol-6-yl)methyl]-4-oxo-4H-pyrido[1,2-a]pyrimidine-2-carboxamide
N-{[2-({[(3-methylcyclobutyl)methyl]amino}methyl)-1H-indol-6-yl]methyl}-4-oxo-4H-pyrido[1,2-a]pyrimidine-2-carboxamide
4-oxo-N-[(2-{[(2,3,3-trimethylbutan-2-yl)amino]methyl}-1H-indol-6-yl)methyl]-4H-pyrido[1,2-a]pyrimidine-2-carboxamide
N-[(2-{[({imidazo[1,2-a]pyridin-2-yl}methyl)amino]methyl}-1H-indol-6-yl)methyl]-4-oxo-4H-pyrido[1,2-a]pyrimidine-2-carboxamide
N-({2-[(3,3-diethylazetidin-1-yl)methyl]-1H-indol-6-yl}methyl)-4-oxo-4H-pyrido[1,2-a]pyrimidine-2-carboxamide
4-oxo-N-[(2-{[(pent-3-yn-1-yl)amino]methyl}-1H-indol-6-yl)methyl]-4H-pyrido[1,2-a]pyrimidine-2-carboxamide
N-{[2-({6-azaspiro[3.4]octan-6-yl}methyl)-1H-indol-6-yl]methyl}-4-oxo-4H-pyrido[1,2-a]pyrimidine-2-carboxamide
N-({2-[(2,2-dimethylpyrrolidin-1-yl)methyl]-1H-indol-6-yl}methyl)-4-oxo-4H-pyrido[1,2-a]pyrimidine-2-carboxamide
N-{[2-({octahydrocyclopenta[c]pyrrol-2-yl}methyl)-1H-indol-6-yl]methyl}-4-oxo-4H-pyrido[1,2-a]pyrimidine-2-carboxamide
N-{[2-({5-azaspiro[2.4]heptanean-5-yl}methyl)-1H-indol-6-yl]methyl}-4-oxo-4H-pyrido[1,2-a]pyrimidine-2-carboxamide
N-[(2-{[({3-methoxybicyclo[1.1.1]pentan-1-yl}methyl)amino]methyl}-1H-indol-6-yl)methyl]-4-oxo-4H-pyrido[1,2-a]pyrimidine-2-carboxamide
4-oxo-N-[(2-{[({spiro[2.2]pentan-1-yl}methyl)amino]methyl}-1H-indol-6-yl)methyl]-4H-pyrido[1,2-a]pyrimidine-2-carboxamide
4-oxo-N-({2-[({spiro[2.3]hexan-1-yl}amino)methyl]-1H-indol-6-yl}methyl)-4H-pyrido[1,2-a]pyrimidine-2-carboxamide
N-[(2-{[({3-cyanobicyclo[1.1.1]pentan-1-yl}methyl)amino]methyl}-1H-indol-6-yl)methyl]-4-oxo-4H-pyrido[1,2-a]pyrimidine-2-carboxamide
N-{[2-({1-oxa-6-azaspiro[3.4]octan-6-yl}methyl)-1H-indol-6-yl]methyl}-4-oxo-4H-pyrido[1,2-a]pyrimidine-2-carboxamide
N-{[2-({2-azaspiro[4.4]nonan-2-yl}methyl)-1H-indol-6-yl]methyl}-4-oxo-4H-pyrido[1,2-a]pyrimidine-2-carboxamide
N-[(2-{[(1-methylcyclopentyl)amino]methyl}-1H-indol-6-yl)methyl]-4-oxo-4H-pyrido[1,2-a]pyrimidine-2-carboxamide
N-{[2-(hydroxymethyl)-1H-indol-6-yl]methyl}-4-oxo-4H-pyrido[1,2-a]pyrimidine-2-carboxamide
N-[(2-{[(1-cyclobutylcyclopropyl)amino]methyl}-1H-indol-6-yl)methyl]-4-oxo-4H-pyrido[1,2-a]pyrimidine-2-carboxamide
N-{[2-({[(1-methylcyclobutyl)methyl]amino}methyl)-1H-indol-6-yl]methyl}-4-oxo-4H-pyrido[1,2-a]pyrimidine-2-carboxamide
4-oxo-N-[(2-{[({spiro[2.3]hexan-5-yl}methyl)amino]methyl}-1H-indol-6-yl)methyl]-4H-pyrido[1,2-a]pyrimidine-2-carboxamide
N-({2-[({[3-(fluoromethyl)bicyclo[1.1.1]pentan-1-yl]methyl}amino)methyl]-1H-indol-6-yl}methyl)-4-oxo-4H-pyrido[1,2-a]pyrimidine-2-carboxamide
N-[(2-{[(1-{3-fluorobicyclo[1.1.1]pentan-1-yl}ethyl)amino]methyl}-1H-indol-6-yl)methyl]-4-oxo-4H-pyrido[1,2-a]pyrimidine-2-carboxamide
N-({2-[(tert-butylamino)methyl]-1H-indol-6-yl}methyl)-4-oxo-4H-pyrido[1,2-a]pyrimidine-2-carboxamide
4-(1-{[2-({2-azaspiro[3.3]heptanean-2-yl}methyl)-1H-indol-6-yl]methyl}-1H-1,2,3-triazol-4-yl)-1H-indazole
N-{[2-(2-{2-azaspiro[3.3]heptanean-2-yl}ethyl)-1H-indol-6-yl]methyl}-4-oxo-4H-pyrido[1,2-a]pyrimidine-2-carboxamide
({3-fluorobicyclo[1.1.1]pentan-1-yl}methyl)({6-[(4-{imidazo[1,5-a]pyridin-8-yl}-1H-1,2,3-triazol-1-yl)methyl]-1H-indol-2-yl}methyl)amine
N-[(2-{[({3-fluorobicyclo[1.1.1]pentan-1-yl}methyl)amino]methyl}-1H-indol-6-yl)methyl]-5-oxo-5H-[1,3]thiazolo[3,2-a]pyrimidine-7-carboxamide
N-[(2-{[({bicyclo[1.1.1]pentan-1-yl}methyl)amino]methyl}-1H-pyrrolo[3,2-b]pyridin-6-yl)methyl]-4-oxo-4H-pyrido[1,2-a]pyrimidine-2-carboxamide
N-[(2-{[({3-fluorobicyclo[1.1.1]pentan-1-yl}methyl)amino]methyl}-1H-pyrrolo[3,2-b]pyridin-6-yl)methyl]-4-oxo-4H-pyrido[1,2-a]pyrimidine-2-carboxamide
N-[(2-{[(cyclobutylmethyl)amino]methyl}-1H-pyrrolo[3,2-b]pyridin-6-yl)methyl]-4-oxo-4H-pyrido[1,2-a]pyrimidine-2-carboxamide
N-[(2-{[({3-methylbicyclo[1.1.1]pentan-1-yl}methyl)amino]methyl}-1H-pyrrolo[3,2-c]pyridin-6-yl)methyl]-4-oxo-4H-pyrido[1,2-a]pyrimidine-2-carboxamide
N-[(2-{[(cyclobutylmethyl)amino]methyl}-1H-pyrrolo[3,2-c]pyridin-6-yl)methyl]-4-oxo-4H-pyrido[1,2-a]pyrimidine-2-carboxamide
N-[(2-{[({bicyclo[1.1.1]pentan-1-yl}methyl)amino]methyl}-1H-pyrrolo[3,2-c]pyridin-6-yl)methyl]-4-oxo-4H-pyrido[1,2-a]pyrimidine-2-carboxamide
N-[(2-{[({3-fluorobicyclo[1.1.1]pentan-1-yl}methyl)amino]methyl}-1H-pyrrolo[3,2-c]63yridine-6-yl)methyl]-4-oxo-4H-pyrido[1,2-a]pyrimidine-2-carboxamide
N-[(2-{[(cyclobutylmethyl)amino]methyl}-1H-indol-6-yl)methyl]-4-oxo-4H,6H,7H,8H,9H-pyrido[1,2-a]pyrimidine-2-carboxamide
(cyclobutylmethyl)({6-[(4-{1H-pyrrolo[2,3-b]pyridin-5-yl}-1H-imidazol-1-yl)methyl]-1H-indol-2-yl}methyl)amine
(cyclobutylmethyl)({6-[(4-{imidazo[1,5-a]pyridin-8-yl}-1H-1,2,3-triazol-1-yl)methyl]-1H-indol-2-yl}methyl)amine
N-[(2-{[(2,2-dimethylpropyl)amino]methyl}-1H-indol-6-yl)methyl]-1H-pyrrolo[2,3-b]pyridine-5-carboxamide
N-[(2-{[(cyclobutylmethyl)amino]methyl}-1H-indol-6-yl)methyl]-1H-pyrrolo[2,3-b]pyridine-5-carboxamide
(cyclobutylmethyl)({6-[(4-{1H-pyrrolo[2,3-b]pyridin-5-yl}-1H-1,2,3-triazol-1-yl)methyl]-1H-indol-2-yl}methyl)amine
N-[[2-(2-azabicyclo[2.2.1]heptanean-2-ylmethyl)-1H-indol-6-yl]methyl]-4-oxo-pyrido[1,2-a]pyrimidine-2-carboxamide
N-[(3-fluoro-1-bicyclo[1.1.1]pentanyl)methyl]-1-[6-[(4-imidazo[1,5-a]pyridin-8-yltriazol-1-yl)methyl]-1H-pyrrolo[3,2-c]pyridin-2-yl]methanamine
(cyclobutylmethyl)[(6-{[1-(1H-indazol-4-yl)-1H-1,2,3-triazol-4-yl]methyl}-1H-indol-2-yl)methyl]amine
[(3,3-difluorocyclobutyl)methyl][(6-{[1-(1H-indazol-4-yl)-1H-1,2,3-triazol-4-yl]methyl}-1H-indol-2-yl)methyl]amine
(cyclobutylmethyl)[(6-{[1-(isoquinolin-4-yl)-1H-1,2,3-triazol-4-yl]methyl}-1H-indol-2-yl)methyl]amine
(cyclobutylmethyl)({6-[(1-{imidazo[1,5-a]pyridin-8-yl}-1H-1,2,3-triazol-4-yl)methyl]-1H-indol-2-yl}methyl)amine
3-[1-({2-[({(Bicyclo[1.1.1]pent-1-yl)methyl}amino)methyl]-1H-indol-6-yl}methyl)-1H-1,2,3-triazol-4-yl]-5-methoxy-2-pyridinecarbonitrile;
3-[1-({2-[({(3-Fluorobicyclo[1.1.1]pent-1-yl)methyl}amino)methyl]-1H-indol-6-yl}methyl)-1H-1,2,3-triazol-4-yl]-5-methoxy-2-pyridinecarbonitrile;
5-Methoxy-3-[1-({2-[({(3-methylbicyclo[1.1.1]pent-1-yl)methyl}amino)methyl]-1H-indol-6-yl}methyl)-1H-1,2,3-triazol-4-yl]-2-pyridinecarbonitrile;
3-{1-[(2-{[(Cyclobutylmethyl)amino]methyl}-1H-indol-6-yl)methyl]-1H-1,2,3-triazol-4-yl}-5-methoxy-2-pyridinecarbonitrile;
3-{1-[(2-{(6-Aza-6-spiro[3.4]octyl)methyl}-1H-indol-6-yl)methyl]-1H-1,2,3-triazol-4-yl}-5-methoxy-2-pyridinecarbonitrile;
3-[1-({2-[(4,4-Dimethyl-1-piperidyl)methyl]-1H-indol-6-yl}methyl)-1H-1,2,3-triazol-4-yl]-5-methoxy-2-pyridinecarbonitrile;
N-((2-((6-azaspiro[3.4]octan-6-yl)methyl)-1H-pyrrolo[3,2-c]pyridin-6-yl)methyl)-4-oxo-4H-pyrido[1,2-a]pyrimidine-2-carboxamide
3-(1-((2-(((cyclobutylmethyl)amino)methyl)-1H-indol-6-yl)methyl)-1H-1,2,3-triazol-4-yl)-5-fluoropicolinonitrile
1-cyclobutyl-N-((6-((4-(5-methoxypyridin-3-yl)-1H-1,2,3-triazol-1-yl)methyl)-1H-indol-2-yl)methyl)methanamine;
5-chloro-3-(1-((2-(((cyclobutylmethyl)amino)methyl)-1H-indol-6-yl)methyl)-1H-1,2,3-triazol-4-yl)picolinonitrile;
2-((6-azaspiro[3.4]octan-6-yl)methyl)-6-((4-(imidazo[1,5-a]pyridin-8-yl)-1H-1,2,3-triazol-1-yl)methyl)-1H-pyrrolo[3,2-c]pyridine;
3-[1-({2-[({(Bicyclo[1.1.1]pent-1-yl)methyl}amino)methyl]-1H-indol-6-yl}methyl)-1H-1,2,3-triazol-4-yl]-5-methoxy-2-pyridinecarbonitrile;
3-[1-({2-[({(3-Fluorobicyclo[1.1.1]pent-1-yl)methyl}amino)methyl]-1H-indol-6-yl}methyl)-1H-1,2,3-triazol-4-yl]-5-methoxy-2-pyridinecarbonitrile;
5-Methoxy-3-[1-({2-[({(3-methylbicyclo[1.1.1]pent-1-yl)methyl}amino)methyl]-1H-indol-6-yl}methyl)-1H-1,2,3-triazol-4-yl]-2-pyridinecarbonitrile;
3-{1-[(2-{[(Cyclobutylmethyl)amino]methyl}-1H-indol-6-yl)methyl]-1H-1,2,3-triazol-4-yl}-5-methoxy-2-pyridinecarbonitrile;
3-{1-[(2-{(6-Aza-6-spiro[3.4]octyl)methyl}-1H-indol-6-yl)methyl]-1H-1,2,3-triazol-4-yl}-5-methoxy-2-pyridinecarbonitrile; and
3-[1-({2-[(4,4-Dimethyl-1-piperidyl)methyl]-1H-indol-6-yl}methyl)-1H-1,2,3-triazol-4-yl]-5-methoxy-2-pyridinecarbonitrile.

### (v) Specific Compounds STM3006, STM3480 and STM3675

One particular compound of formula I defined above that is disclosed in International Patent Publication No. WO2020/050898 is STM3006, which is 6-bromo-4-[1-({6-[(4,4-dimethylpiperidin-1-yl)methyl]imidazo[1,2-a]pyridin-2-yl}methyl)-1H-1,2,3-triazol-4-yl]-1H-indazole, and has the structure shown below

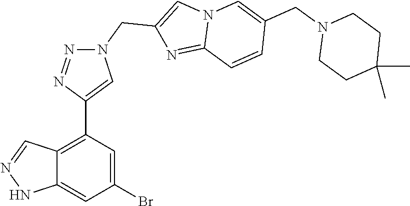 [SMILES: CC1(C)CCN(Cc2ccc3nc(Cn4cc(-c5cc(Br)cc6[nH]ncc56)nn4)cn3c2)CC1]

Two particular compounds of Formulae II, VI and VII defined above are STM3480 and STM3675.

STM3480 is N-[(2-{[(cyclobutylmethyl)amino]methyl}-1H-indol-6-yl)methyl]-4-oxo-4H-pyrido[1,2-a]pyrimidine-2-carboxamide, which has the structure shown below:

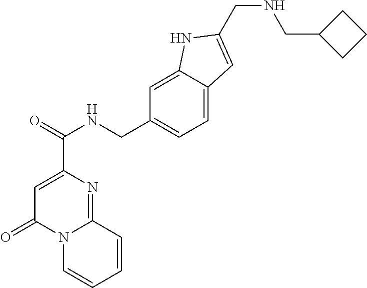 [SMILES: O=C(NCc1ccc2cc(CNCC3CCC3)[nH]c2c1)c1cc(=O)n2ccccc2n1]

STM3675 is N-[(2-{[({3-fluorobicyclo[1.1.1]pentan-1-yl}methyl)amino]methyl}-1H-indol-6-yl)methyl]-4-oxo-4H-pyrido[1,2-a]pyrimidine-2-carboxamide, the structure of which is also depicted below:

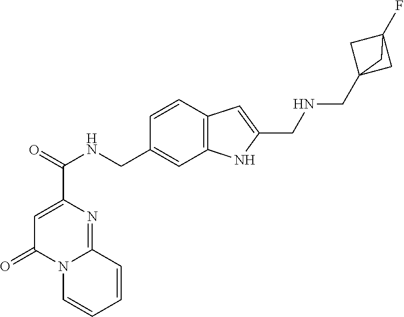 [SMILES: O=C(NCc1ccc2cc(CNCC34CC(F)(C3)C4)[nH]c2c1)c1cc(=O)n2ccccc2n1]

The METTL3 inhibitors used in the combination therapies of the present invention may be any METTL3 inhibitor.

In a particular embodiment, the METTL3 inhibitor is a compound of formula I, II, VI or VII defined herein, or a pharmaceutically acceptable salt thereof.

In another embodiment, the METTL3 inhibitor is any one of the specific compounds listed list 1 and/or list 2 above, or a pharmaceutically acceptable salt thereof.

In another embodiment, the METTL3 inhibitor is any one of the specific compounds STM 3006, STM 3480, or STM3675 identified above, or a pharmaceutically acceptable salt thereof.

In a particular embodiment, the METTL3 inhibitor is STM3480 identified above, or a pharmaceutically acceptable salt thereof.

1. Combinations of METTL3 Inhibitors Defined Herein with Immuno-Oncology Agents (e.g. Immune Checkpoint Inhibitors)

One aspect of the present invention resides in the recognition that the METTL3 inhibitor compound, STM3480, is particularly suited to use in combination with immune checkpoint inhibitors (see Examples 1 and 2).

Immune checkpoint inhibitors are a class of anticancer agents that have shown great promise in some cancer patients. The inhibition of immune checkpoints (e.g. CTLA4, LAG3, PD1 or PD-L1 inhibitors) results in the enhancement of the immune response to a tumour. However, some cancers do not respond sufficiently well to immune checkpoint inhibitor therpay alone, so there is a need for improved treatment strategies.

The inventors have surprisingly discovered that the METTL3 inhibitor compound STM3480 can significantly potentiate the therapeutic effects of immune checkpoint inhibitors. In an embodiment, the METTL3 inhibitor compound STM3480 synergistically potentiates the therapeutic effects of immune checkpoint inhibitors (and vice versa), thereby rendering the tumours more susceptible to the combination therapy (i.e. the therapeutic effect observed is greater than the additive effect of the two agents individually).

Thus, the combination treatment of the present invention has the potential to provide better therapeutic outcomes in cancer patients, especially cancer patients that do not respond well to therapy with a METTL3 inhibitor or an immune checkpoint inhibitor alone.

In one aspect, the present invention provides a combination comprising a METTL3 inhibitor as defined herein (e.g. STM3480), or a pharmaceutically acceptable salt thereof, and an immune checkpoint inhibitor, or a pharmaceutically acceptable salt thereof.

In another aspect, the present invention provides a pharmaceutical product comprising a combination of a METTL3 inhibitor as defined herein (e.g. STM3480), or a pharmaceutically acceptable salt thereof, and an immune checkpoint inhibitor, or a pharmaceutically acceptable salt thereof.

In one embodiment, the pharmaceutical product may comprise a kit of parts comprising separate formulations of a METTL3 inhibitor as defined herein (e.g. STM3480), or a pharmaceutically acceptable salt thereof, and an immune checkpoint inhibitor, or a pharmaceutically acceptable salt thereof. The separate formulations of a METTL3 inhibitor as defined herein (e.g. STM3480), or a pharmaceutically acceptable salt thereof, and an immune checkpoint inhibitor, or a pharmaceutically acceptable salt thereof, may be administered sequentially, separately and/or simultaneously.

In another embodiment the pharmaceutical product is a kit of parts which comprises:
a first container comprising a METTL3 inhibitor as defined herein (e.g. STM3480), or a pharmaceutically acceptable salt thereof, in association with a pharmaceutically acceptable adjuvant, diluent or carrier; and
a second container comprising an immune checkpoint inhibitor, or a pharmaceutically acceptable salt thereof, in association with a pharmaceutically acceptable adjuvant, diluent or carrier, and
a container means for containing said first and second containers.

In one embodiment, the pharmaceutical product may comprise a one or more unit dosage forms (e.g. vials, tablets or capsules in a blister pack). In one embodiment, each unit dose comprises only one agent selected from the a METTL3 inhibitor as defined herein (e.g. STM3480) compound and the immune checkpoint inhibitor. In another embodiment, the unit dosage form comprises both the a METTL3 inhibitor as defined herein (e.g. STM3480) compound and the immune checkpoint inhibitor.

In one embodiment the pharmaceutical product or kit of parts further comprises means for facilitating compliance with a dosage regimen, for instance instructions detailing how to administer the combination.

In one embodiment, the pharmaceutical product or kit of parts further comprises instructions indicating that the combination, as defined herein, can be used in the treatment of cancer.

In one embodiment, the pharmaceutical product is a pharmaceutical composition.

### Immune Checkpoint Inhibitors

Any immune checkpoint inhibitor may be used in the combination therapy defined herein.

In one embodiment, the immune checkpoint inhibitor is selected from a PD1, PD-L1 inhibitor, a LAG3 inhibitor and a CTLA-4 inhibitor. In a particular embodiment, the immune checkpoint inhibitor is a PD1 or PD-L1 inhibitor.

PD-1 is a cell surface receptor protein present on T cells. PD-1 plays an important role in down-regulating the immune system and promoting self-tolerance by suppressing T cell inflammatory activity. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD1 binds two ligands, PD-L1 and PD-L2. PD-L1 is of particular interest as it is highly expressed in several cancers and hence the role of PD1 in cancer immune evasion is well established. Monoclonal antibodies targeting PD-1 that boost the immune system are being developed for the treatment of cancer. Many tumour cells express PD-L1, an immunosuppressive PD-1 ligand; inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda) and nivolumab (Opdivo). These drugs have been shown to be effective in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (Imfinzi). These drugs have also been shown to be helpful in treating different types of cancer, including bladder cancer, non-small cell lung cancer, and Merkel cell skin cancer (Merkel cell carcinoma). They are also being studied for use against other types of cancer.

Examples of LAG3 inhibitors include BMS-986016/Relatlimab, TSR-033, REGN3767, MGD013 (bispecific DART binding PD-1 and LAG-3), GSK2831781 and LAG525.

Examples of CTLA-4 inhibitors include MDX-010/Ipilimumab, AGEN1884, and CP-675,206/Tremelimumab.

In one embodiment, the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab and durvalumab, or a pharmaceutically acceptable salt thereof.

In another embodiment, the immune checkpoint inhibitor is selected from BMS-986016/Relatlimab, MDX-010/Ipilimumab, CP-675,206/Tremelimumab, pembrolizumab, nivolumab, atezolizumab, avelumab, and durvalumab, or a pharmaceutically acceptable salt thereof.

In another embodiment, the immune checkpoint inhibitor is selected from pembrolizumab, nivolumab, atezolizumab, avelumab and durvalumab, or a pharmaceutically acceptable salt thereof.

In another embodiment, the immune checkpoint inhibitor is selected from pembrolizumab, nivolumab, atezolizumab, avelumab and durvalumab, or a pharmaceutically acceptable salt thereof.

In another embodiment, the immune checkpoint inhibitor is selected from pembrolizumab and avelumab, or a pharmaceutically acceptable salt thereof.

### Therapeutic Uses

Thus, the present invention relates, in one aspect, to a METTL3 inhibitor as defined herein, or a pharmaceutically acceptable salt thereof, for use as an immune-sensitiser.

The present invention also relates to the use of a METTL3 inhibitor as defined herein, or a pharmaceutically acceptable salt thereof, in the manufacture of a medicament for use as an immune-sensitiser.

The present invention also relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is administered in combination with an immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BITE), oncolytic viruses, cancer vaccines, and/or CAR-T cell therapy).

The present invention also relates to the use of a METTL3 inhibitor as defined herein, or a pharmaceutically acceptable salt thereof, in the manufacture of a medicament for use in the treatment of cancer, wherein the METTL3 inhibitor as defined herein, or a pharmaceutically acceptable salt thereof, is administered in combination with an immune oncology agent or therapy (e.g. immune checkpoint inhibitors (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BITE), oncolytic viruses, cancer vaccines, and/or CAR-T cell therapy).

The present invention also relates to a method of treating cancer, the method comprising administering a therapeutically effective amount of a METTL3 inhibitor as defined herein, or a pharmaceutically acceptable salt thereof, to a patient in combination with an immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BiTE), oncolytic viruses, cancer vaccines, and/or CAR-T cell therapy).

Suitably, the METTL3 inhibitor as defined herein, or a pharmaceutically acceptable salt thereof, is administered simultaneously, sequentially or separately with the immune oncology agent or therapy (e.g. immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A2AR antagonists, Bispecific T cell engagers (BiTE), oncolytic viruses, cancer vaccines, and/or CAR-T cell therapy).

Suitably, the immune oncology agent is an immune checkpoint inhibitor (e.g. a PD1, PD-L1 inhibitor, LAG3 or CTLA-4 inhibitor).

In another aspect, the present invention relates to a combination as defined herein, or a pharmaceutical product as defined herein, or a pharmaceutical composition as defined herein for use in therapy.

In another aspect, the present invention relates to a combination as defined herein, or a pharmaceutical product as defined herein, or a pharmaceutical composition as defined herein for use in the treatment of cancer.

In another aspect, the present invention relates to a use of a combination as defined herein in the manufacture of a medicament for the treatment of cancer.

In another aspect, the present invention relates to a method of treating of cancer in a subject in need thereof comprising administering to said subject a therapeutically effective amount of a combination as defined herein.

In another aspect, the present invention relates to a method of potentiating the immune response to a tumour, the method comprising administering to a patient in need of such treatment a therapeutically effective amount of a combination as defined herein.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with an immune checkpoint inhibitor, or a pharmaceutically acceptable salt thereof.

In another aspect, the present invention relates to an immune checkpoint inhibitor, or a pharmaceutically acceptable salt thereof, for use in the treatment of cancer, wherein the immune checkpoint inhibitor is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with an immune checkpoint inhibitor, or a pharmaceutically acceptable salt thereof.

In another aspect, the present invention relates to a use of an immune checkpoint inhibitor, or a pharmaceutically acceptable salt thereof in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and an immune checkpoint inhibitor, or a pharmaceutically acceptable salt thereof, wherein the METTL3 inhibitor as defined herein, or a pharmaceutically acceptable salt thereof, and the immune checkpoint inhibitor, or a pharmaceutically acceptable salt thereof are administered sequentially, separately or simultaneously with one another.

In another aspect, the present invention relates to a method of treating cancer or potentiating the effect of an immune checkpoint inhibitor, or a pharmaceutically acceptable salt thereof, the method comprising administering to a patient in need of such treatment a therapeutically effective amount the immune checkpoint inhibitor, or a pharmaceutically acceptable salt thereof, separately, sequentially or simultaneously with a METTL3 inhibitor as defined herein, or a pharmaceutically acceptable salt thereof.

In another aspect, the present invention relates to a method of treating cancer or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with an immune checkpoint inhibitor, or a pharmaceutically acceptable salt thereof.

In another aspect, the present invention relates to a method of potentiating the immune response to a tumour, the method comprising administering to a patient in need of such treatment a therapeutically effective amount of a METTL3 inhibitor as defined herein, or a pharmaceutically acceptable salt thereof, and an immune checkpoint inhibitor, or a pharmaceutically acceptable salt thereof, wherein the METTL3 inhibitor as defined herein, or a pharmaceutically acceptable salt thereof, and the immune checkpoint inhibitor, or a pharmaceutically acceptable salt thereof are administered sequentially, separately or simultaneously with one another.

The term “cancer” is used herein to refer to an unwanted, uncontrolled and abnormal malignant cellular proliferation, whether in vitro or in vivo. The term includes benign, pre-malignant and malignant cellular proliferation. Any type of cell may be treated, including but not limited to, lung, colon, breast, ovarian, prostate, liver, pancreas, brain, bladder, kidney, bone, nerves and skin.

The anti-proliferative effects of the combination therapy of the present invention has particular application in the treatment of human cancers. In particular, the combination therapy of the present invention will be useful for treating any human cancer in which METTL3 and/or immune checkpoint activity is implicated. This includes any cancer that has been unresponsive to therapy comprising either a METTL3 inhibitor or immune checkpoint inhibitor alone.

In an embodiment of the invention, the anti-tumour effects of the combination therapy of the present invention has particular application in the treatment and/or prevention of a wide range of cancers including, but not limited to, non-solid tumours such as leukaemia, for example acute myeloid leukaemia, multiple myeloma, haematologic malignancies or lymphoma, and also solid tumours and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostate, breast, renal, testicular, ovarian, skin, cervical, lung, muscle, neuronal, oesophageal, bladder, lung, uterine, vulval, endometrial, kidney, colorectal, pancreatic, pleural/peritoneal membranes, salivary gland, and epidermoid tumours and haematological malignancies.

Suitably, the cancer is a solid tumour.

In one embodiment the cancer is selected from lung, colon, rectal, breast, ovarian, bladder, kidney, prostate, liver, pancreas, brain, bone, blood and skin cancer.

In one embodiment the cancer is a human cancer. Suitably, the human cancer is selected from lung, colon, breast, ovarian, bladder, kidney, prostate, liver, pancreas, brain, bone, blood and skin cancer. In one embodiment, the human cancer is selected from glioblastoma, lung cancer, breast cancer, renal cell carcinoma and Hodgkin lymphoma.

The anti-cancer effect may arise through one or more mechanisms, including but not limited to, the promotion of an antitumour immune response, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures or within an organ), or the promotion of apoptosis (programmed cell death).

As indicated above, the immune checkpoint inhibitor may be any immune checkpoint inhibitor as defined in any of the embodiments herein and the METTL3 inhibitor may be any known METTL3 inhibitor.

In one embodiment, the immune checkpoint inhibitor is selected from BMS-986016/Relatlimab, TSR-033, REGN3767, MGD013 (bispecific DART binding PD-1 and LAG-3), GSK2831781, LAG525, and MDX-010/Ipilimumab, AGEN1884, CP-675,206/Tremelimumab, pembrolizumab, nivolumab, atezolizumab, avelumab and durvalumab, or a pharmaceutically acceptable salt thereof; and the METTL3 inhibitor is as defined herein.

In another embodiment, the immune checkpoint inhibitor is selected from BMS-986016/Relatlimab, MDX-010/Ipilimumab, CP-675,206/Tremelimumab, pembrolizumab, nivolumab, atezolizumab, avelumab, and durvalumab, or a pharmaceutically acceptable salt thereof; and the METTL3 inhibitor is selected from STM3006, STM3480 or STM3675, or a pharmaceutically-acceptable salt thereof, as defined herein.

In another embodiment, the immune checkpoint inhibitor is selected from pembrolizumab, nivolumab, atezolizumab, avelumab and durvalumab, or a pharmaceutically acceptable salt thereof or a pharmaceutically acceptable salt thereof; and the METTL3 inhibitor is selected from STM3006, STM3480 or STM3675, or a pharmaceutically-acceptable salt thereof, as defined herein.

Suitably, the METTL3 inhibitor is STM3480.

2. Combinations of METTL3 Inhibitors with BCL2 Inhibitors (e.g. Venetoclax)

One aspect of the present invention resides in the recognition that the METTL3 inhibitor compounds, STM3480, STM3006 and STM3675, when administered in combination with a BCL2 inhibitor (venetoclax), produced a synergistic increase in potency (see Example 3).

Thus, the administration of a METTL3 inhibitor synergistically enhances the antitumour effect of BCL2 inhibitor (e.g. venetoclax) therapy and vice versa. The combination of a METTL3 inhibitor with a BCL2 inhibitor (e.g. venetoclax) therefore offers a promising therapy for diseases or conditions in which BCL2 inhibitor therapy is beneficial (e.g. the treatment of cancers including acute myeloid leukaemia (AML), chronic lymphocytic leukaemia (CLL), small lymphocytic lymphoma (SLL) and myelodysplastic syndromes (MDS)).

The combination treatment of the present invention has the potential to provide better therapeutic outcomes in cancer patients, especially cancer patients that do not respond well to therapy with a METTL3 inhibitor or a BCL2 inhibitor (e.g. venetoclax) alone.

In one aspect, the present invention provides a combination comprising a METTL3 inhibitor as defined herein (e.g. STM3480, STM3006 and STM3675), or a pharmaceutically acceptable salt thereof, and a BCL2 inhibitor (e.g. venetoclax), or a pharmaceutically acceptable salt thereof.

In another aspect, the present invention provides a pharmaceutical product comprising a combination of a METTL3 inhibitor as defined herein (e.g. STM3480, STM3006 and STM3675), or a pharmaceutically acceptable salt thereof, and a BCL2 inhibitor (e.g. venetoclax), or a pharmaceutically acceptable salt thereof.

In one embodiment, the pharmaceutical product may comprise a kit of parts comprising separate formulations of a METTL3 inhibitor as defined herein (e.g. STM3480, STM3006 and STM3675), or a pharmaceutically acceptable salt thereof, and a BCL2 inhibitor (e.g. venetoclax), or a pharmaceutically acceptable salt thereof. The separate formulations of a METTL3 inhibitor as defined herein (e.g. STM3480, STM3006 and STM3675), or a pharmaceutically acceptable salt thereof, and a BCL2 inhibitor (e.g. venetoclax), or a pharmaceutically acceptable salt thereof, may be administered sequentially, separately and/or simultaneously.

In another embodiment the pharmaceutical product is a kit of parts which comprises:
a first container comprising a METTL3 inhibitor as defined herein (e.g. STM3480, STM3006 and STM3675), or a pharmaceutically acceptable salt thereof, in association with a pharmaceutically acceptable adjuvant, diluent or carrier; and
a second container comprising a BCL2 inhibitor (e.g. venetoclax), or a pharmaceutically acceptable salt thereof, in association with a pharmaceutically acceptable adjuvant, diluent or carrier, and
a container means for containing said first and second containers.

In one embodiment, the pharmaceutical product may comprise a one or more unit dosage forms (e.g. vials, tablets or capsules in a blister pack). In one embodiment, each unit dose comprises only one agent selected from the a METTL3 inhibitor as defined herein (e.g. STM3480, STM3006 and STM3675) compound and the BCL2 inhibitor (e.g. venetoclax). In another embodiment, the unit dosage form comprises both the a METTL3 inhibitor as defined herein (e.g. STM3480, STM3006 and STM3675) compound and the BCL2 inhibitor (e.g. venetoclax).

In one embodiment the pharmaceutical product or kit of parts further comprises means for facilitating compliance with a dosage regimen, for instance instructions detailing how to administer the combination.

In one embodiment, the pharmaceutical product or kit of parts further comprises instructions indicating that the combination, as defined herein, can be used in the treatment of cancer.

In one embodiment, the pharmaceutical product is a pharmaceutical composition.

### The BCL2 Inhibitor

Any suitable BCL2 inhibitor approved for therapeutic use may be used in this combination therapy of the present invention

Suitably, the BCL2 inhibitor is venetoclax, or a pharmaceutically acceptable salt thereof. The chemical name for ventoclax is 4-{4-[(4′-chloro-5,5-dimethyl[3,4,5,6-tetrahydro[1,1′-biphenyl]]-2-yl)methyl]piperazin-1-yl}-N-(3-nitro-4-{[(oxan-4-yl)methyl]amino}benzene-1-sulfonyl)-2-[(1H-pyrrolo[2,3-b]pyridin-5-yl)oxy]benzamide, and its chemical structure is shown below:

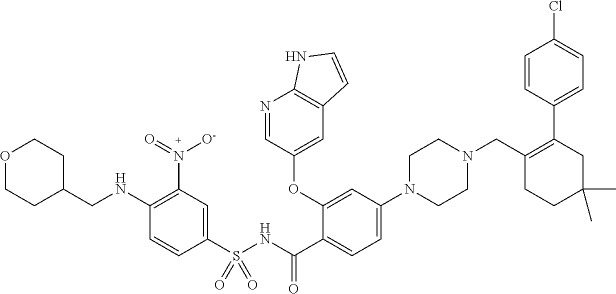

### Therapeutic Uses

The present invention also relates to a METTL3 inhibitor as defined herein, or a pharmaceutically acceptable salt thereof, for use in the treatment of diseases or conditions in which BCL2 inhibitor therapy is beneficial (e.g. the treatment of cancer), wherein the METTL3 inhibitor as defined herein, or a pharmaceutically acceptable salt thereof, is administered in combination with a BCL2 inhibitor (e.g. venetoclax).

The present invention also relates to the use of a METTL3 inhibitor as defined herein, or a pharmaceutically acceptable salt thereof, in the manufacture of a medicament for use in the treatment of diseases or conditions in which BCL2 inhibitor therapy is beneficial (e.g. the treatment of cancer), wherein the METTL3 inhibitor as defined herein, or a pharmaceutically acceptable salt thereof, is administered in combination with a BCL2 inhibitor (e.g. venetoclax).

The present invention also relates to a method of treating diseases or conditions in which BCL2 inhibitor therapy is beneficial (e.g. the treatment of cancer), the method comprising administering a therapeutically effective amount of a METTL3 inhibitor as defined herein, or a pharmaceutically acceptable salt thereof, to a patient in combination with a BCL2 inhibitor (e.g. venetoclax).

Suitably, the METTL3 inhibitor as defined herein, or a pharmaceutically acceptable salt thereof, is administered simultaneously, sequentially or separately with the BCL2 inhibitor (e.g. venetoclax) therapy.

In one aspect the present invention relates to a combination comprising a METTL3 inhibitor as defined herein, or a pharmaceutically acceptable salt thereof, and a BCL2 inhibitor (e.g. venetoclax), or a pharmaceutically acceptable salt thereof.

In another aspect the present invention relates to a pharmaceutical product comprising a combination as defined above.

In another aspect, the present invention relates to a pharmaceutical composition comprising a combination as defined above, and one or more pharmaceutically acceptable excipients.

In another aspect, the present invention relates to a combination as defined above, or a pharmaceutical product as defined above, or a pharmaceutical composition as defined above for use in therapy.

In another aspect, the present invention relates to a combination as defined herein, or a pharmaceutical product as defined above, or a pharmaceutical composition as defined above for use in the treatment of diseases or conditions in which BCL2 inhibitor therapy is beneficial (e.g. the treatment of cancer).

In another aspect, the present invention relates to the use of a combination as defined above in the manufacture of a medicament for the treatment of diseases or conditions in which BCL2 inhibitor therapy is beneficial (e.g. the treatment of cancer).

In another aspect, the present invention relates to a method of treating of diseases or conditions in which BCL2 inhibitor therapy is beneficial (e.g. the treatment of cancer) in a subject in need thereof comprising administering to said subject a therapeutically effective amount of a combination as defined above.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with a BCL2 inhibitor (e.g. venetoclax), or a pharmaceutically acceptable salt thereof.

In another aspect, the present invention relates to a BCL2 inhibitor (e.g. venetoclax), or a pharmaceutically acceptable salt thereof, for use in the treatment of cancer, wherein the BCL2 inhibitor (e.g. venetoclax), or a pharmaceutically acceptable salt thereof, is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with a BCL2 inhibitor (e.g. venetoclax), or a pharmaceutically acceptable salt thereof.

In another aspect, the present invention relates to a use of a BCL2 inhibitor (e.g. venetoclax), or a pharmaceutically acceptable salt thereof, in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and a BCL2 inhibitor (e.g. venetoclax), or a pharmaceutically acceptable salt thereof, wherein the METTL3 inhibitor as defined herein, or a pharmaceutically acceptable salt thereof, and the BCL2 inhibitor (e.g. venetoclax), or a pharmaceutically acceptable salt thereof, are administered sequentially, separately or simultaneously with one another.

In another aspect, the present invention relates to a method of treating cancer, or potentiating the effect of a BCL2 inhibitor (e.g. venetoclax), or a pharmaceutically acceptable salt thereof, the method comprising administering to a patient in need of such treatment a therapeutically effective amount the BCL2 inhibitor (e.g. venetoclax), or a pharmaceutically acceptable salt thereof, separately, sequentially or simultaneously with a METTL3 inhibitor as defined herein, or a pharmaceutically acceptable salt thereof.

In another aspect, the present invention relates to a method of treating cancer or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with a BCL2 inhibitor (e.g. venetoclax), or a pharmaceutically acceptable salt thereof.

The anti-proliferative effects of the combination therapy of the present invention has particular application in the treatment of human cancers. In particular, the combination therapy of the present invention will be useful for treating any human cancer in which METTL3 and/or BCL2 activity is implicated. This includes any cancer that has been unresponsive to therapy comprising either a METTL3 inhibitor or BCL2 inhibitor alone.

Suitably, the cancer is selected from acute myeloid leukaemia (AML), chronic lymphocytic leukaemia (CLL), small lymphocytic lymphoma (SLL) and myelodysplastic syndromes (MDS).

In an embodiment, this combination therapy of the invention is suitable for the treatment of acute myeloid leukaemia (AML), chronic lymphocytic leukaemia (CLL), small lymphocytic lymphoma (SLL).

The anti-cancer effect may arise through one or more mechanisms, including but not limited to, the promotion of an antitumour immune response, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures or within an organ), or the promotion of apoptosis (programmed cell death).

As indicated above, the BCL2 inhibitor may be any BCL2 inhibitor and the METTL3 inhibitor may be any known METTL3 inhibitor.

In one embodiment, the BCL2 inhibitor is venetoclax, or a pharmaceutically acceptable salt thereof; and the METTL3 inhibitor is as defined herein.

In one embodiment, the BCL2 inhibitor is venetoclax, or a pharmaceutically acceptable salt thereof; and the METTL3 inhibitor is selected any compound of the formulae I, II, VI or VII defined above, or a pharmaceutically acceptable salt thereof.

In one embodiment, the BCL2 inhibitor is venetoclax, or a pharmaceutically acceptable salt thereof; and the METTL3 inhibitor is selected from STM3006, STM3480 or STM3675, or a pharmaceutically-acceptable salt thereof.

In one embodiment, the BCL2 inhibitor is venetoclax, or a pharmaceutically acceptable salt thereof; and the METTL3 inhibitor is STM3480, or a pharmaceutically-acceptable salt thereof.

3. Combinations of METTL3 Inhibitors with Anthracycline Topoisomerase 2 Inhibitors (e.g. Daunorubicin), Cytarabine, Hypomethylating Agents (e.g. 5-Azacitidine), FLT3 Inhibitors (e.g. Quizartinib) and Decitabine

One aspect of the present invention resides in the recognition that the METTL3 inhibitor compounds, STM3480 and STM3006, when administered in combination with daunorubicin, cytarabine, 5-azacitidine and quizartinib, produced an enhanced therapeutic effect in Kasumi1 or MOLM-14 AML cell lines (see Example 4).

Collectively, these data suggest that the administration of a METTL3 inhibitor enhances the antitumour effects of these AML standard of care agents. Thus, the combination of a METTL3 inhibitor with either an anthracycline topoisomerase 2 inhibitors (e.g. daunorubicin), cytarabine, hypomethylating agents (e.g. 5-azacitidine or decitabine) and/or FLT3 inhibitors (e.g. quizartinib) offers a promising therapy for the treatment of cancer (and especially acute myeloid leukaemia (AML), chronic lymphocytic leukaemia (CLL), small lymphocytic lymphoma (SLL) and/or Myelodysplastic syndromes (MDS)).

This combination treatment of the present invention therefore has the potential to provide better therapeutic outcomes in cancer patients, especially cancer patients that do not respond well to therapy with a METTL3 inhibitor or an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib).

In one aspect, the present invention provides a combination comprising a METTL3 inhibitor as defined herein (e.g. STM3480, STM3006 and STM3675), or a pharmaceutically acceptable salt thereof, and an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib), or a pharmaceutically acceptable salt thereof.

In another aspect, the present invention provides a pharmaceutical product comprising a combination of a METTL3 inhibitor as defined herein (e.g. STM3480, STM3006 and STM3675), or a pharmaceutically acceptable salt thereof, and an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib), or a pharmaceutically acceptable salt thereof.

In one embodiment, the pharmaceutical product may comprise a kit of parts comprising separate formulations of a METTL3 inhibitor as defined herein (e.g. STM3480, STM3006 and STM3675), or a pharmaceutically acceptable salt thereof, and an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib), or a pharmaceutically acceptable salt thereof. The separate formulations of a METTL3 inhibitor as defined herein and an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib), or a pharmaceutically acceptable salt thereof, may be administered sequentially, separately and/or simultaneously.

In another embodiment the pharmaceutical product is a kit of parts which comprises:
a first container comprising a METTL3 inhibitor as defined herein (e.g. STM3480, STM3006 and STM3675), or a pharmaceutically acceptable salt thereof, in association with a pharmaceutically acceptable adjuvant, diluent or carrier; and
a second container comprising an anthracycline topoisomerase 2 inhibitor (e.g. daunorubicin), cytarabine, a hypomethylating agent (e.g. 5-azacitidine or decitabine) and/or a FLT3 inhibitor (e.g. quizartinib), or a pharmaceutically acceptable salt thereof, in association with a pharmaceutically acceptable adjuvant, diluent or carrier, and
a container means for containing said first and second containers.

In one embodiment, the pharmaceutical product may comprise a one or more unit dosage forms (e.g. vials, tablets or capsules in a blister pack). In one embodiment, each unit dose comprises only one agent selected from the a METTL3 inhibitor as defined herein (e.g. STM3480, STM3006 and STM3675) and the anthracycline topoisomerase 2 inhibitor (e.g. daunorubicin), cytarabine, hypomethylating agent (e.g. 5-azacitidine or decitabine) and/or FLT3 inhibitor (e.g. quizartinib). In another embodiment, the unit dosage form comprises both the a METTL3 inhibitor as defined herein (e.g. STM3480, STM3006 and STM3675) and the anthracycline topoisomerase 2 inhibitor (e.g. daunorubicin), cytarabine hypomethylating agent (e.g. 5-azacitidine or decitabine) and/or FLT3 inhibitor (e.g. quizartinib).

In one embodiment the pharmaceutical product or kit of parts further comprises means for facilitating compliance with a dosage regimen, for instance instructions detailing how to administer the combination.

In one embodiment, the pharmaceutical product or kit of parts further comprises instructions indicating that the combination, as defined herein, can be used in the treatment of cancer.

In one embodiment, the pharmaceutical product is a pharmaceutical composition.

### The Anthracycline Topoisomerase 2 Inhibitors, Hypomethylating Agents and FLT3 Inhibitors

Any suitable anthracycline topoisomerase 2 inhibitors, hypomethylating agents and FLT3 inhibitors may be used in this combination therapy of the present invention.

Suitably, the anthracycline topoisomerase 2 inhibitor is selected from daunorubicin, doxorubicin, epirubicin or idarubicin. Most suitably, the anthracycline topoisomerase 2 inhibitor is selected from daunorubicin and doxorubicin. In an embodiment, the anthracycline topoisomerase 2 inhibitor is daunorubicin.

Suitably, the hypomethylating agent is selected from 5-azacitidine or decitabine. In an embodiment, the hypomethylating agent is 5-azacitidine. In another embodiment, the hypomethylating agent is decitabine.

Suitably, the FLT3 inhibitor is selected from sorafenib, lestaurtinib, midostaurin, quizartinib, crenolanib, or gilteritinib. More suitably, the FLT3 inhibitor is selected from midostaurin, quizartinib or gilteritinib. In an embodiment, the FLT3 inhibitor is midostaurin. In another embodiment, the FLT3 inhibitor is quizartinib. In another embodiment, the FLT3 inhibitor is gilteritinib.

### Therapeutic Uses

The present invention also relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is administered in combination with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

The present invention also relates to the use of a METTL3 inhibitor as defined herein, or a pharmaceutically acceptable salt thereof, in the manufacture of a medicament for use in the treatment of cancer, wherein the METTL3 inhibitor as defined herein, or a pharmaceutically acceptable salt thereof, is administered in combination with one or more additional agents selected from
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

The present invention also relates to a method of treating cancer, the method comprising administering a therapeutically effective amount of a METTL3 inhibitor as defined herein, or a pharmaceutically acceptable salt thereof, to a patient in combination with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

Suitably, the METTL3 inhibitor as defined herein, or a pharmaceutically acceptable salt thereof, is administered simultaneously, sequentially or separately with the:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect the present invention relates to a pharmaceutical product comprising a combination as defined above.

In another aspect, the present invention relates to a pharmaceutical composition comprising a combination as defined above, and one or more pharmaceutically acceptable excipients.

In another aspect, the present invention relates to a combination as defined above, or a pharmaceutical product as defined above, or a pharmaceutical composition as defined above for use in therapy.

In another aspect, the present invention relates to a combination as defined above, or a pharmaceutical product as defined above, or a pharmaceutical composition as defined above for use in the treatment of cancer.

In another aspect, the present invention relates to the use of a combination as defined above in the manufacture of a medicament for the treatment of cancer.

In another aspect, the present invention relates to a method of treating of cancer in a subject in need thereof comprising administering to said subject a therapeutically effective amount of a combination as defined above.

In another aspect, the present invention relates to a METTL3 inhibitor as defined herein, or a pharmaceutically acceptable salt thereof, for use in the treatment of cancer, wherein the METTL3 inhibitor as defined herein, or a pharmaceutically acceptable salt thereof, is for simultaneous, separate or sequential administration with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect, the present invention relates to an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, for use in the treatment of cancer, wherein the agent, or a pharmaceutically acceptable salt thereof, is for simultaneous, separate or sequential administration with a METTL3 inhibitor as defined herein, or a pharmaceutically acceptable salt thereof.

In another aspect, the present invention relates to a use of a METTL3 inhibitor as defined herein, or a pharmaceutically acceptable salt thereof, in the manufacture of a medicament for treating cancer, wherein the medicament is for simultaneous, separate or sequential administration with one or more additional agents selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

In another aspect, the present invention relates to a use of an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, in the manufacture of a medicament for treating cancer, wherein the medicament is for simultaneous, separate or sequential administration with a METTL3 inhibitor as defined herein, or a pharmaceutically acceptable salt thereof.

In another aspect, the present invention relates to a method of treating cancer comprising administering to a subject in need thereof a therapeutically effective amount of a METTL3 inhibitor as defined herein, or a pharmaceutically acceptable salt thereof, and an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof,
   wherein the METTL3 inhibitor as defined herein, or a pharmaceutically acceptable salt thereof, and the agent, or a pharmaceutically acceptable salt thereof, are administered sequentially, separately or simultaneously with one another.

In another aspect, the present invention relates to a method of treating cancer, or potentiating the effect of an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof, the method comprising administering to a patient in need of such treatment a therapeutically effective amount the agent, or a pharmaceutically acceptable salt thereof, separately, sequentially or simultaneously with a METTL3 inhibitor as defined herein, or a pharmaceutically acceptable salt thereof.

In another aspect, the present invention relates to a method of treating cancer, or potentiating the effect of a METTL3 inhibitor as defined herein, or a pharmaceutically acceptable salt thereof, the method comprising administering to a patient in need of such treatment a therapeutically effective amount of the METTL3 inhibitor as defined herein, or a pharmaceutically acceptable salt thereof, separately, sequentially or simultaneously with an agent selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

The anti-proliferative effects of the combination therapy of the present invention has particular application in the treatment of human cancers.

Suitably, the cancer is selected from acute myeloid leukaemia (AML), chronic lymphocytic leukaemia (CLL), small lymphocytic lymphoma (SLL) and myelodysplastic syndromes (MDS).

In an embodiment, this combination therapy of the invention is suitable for the treatment of acute myeloid leukaemia (AML), chronic lymphocytic leukemia (CLL), small lymphocytic lymphoma (SLL).

The anti-cancer effect may arise through one or more mechanisms, including but not limited to, the promotion of an antitumour immune response, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures or within an organ), or the promotion of apoptosis (programmed cell death).

As indicated above, any suitable anthracycline topoisomerase 2 inhibitor, hypomethylating agent and FLT3 inhibitor may be used in the combination therapy of the present invention.

In an embodiment, the METTL3 inhibitor is as defined herein and the second agent is selected from daunorubicin, doxorubicin, epirubicin, idarubicin, 5-azacitidine, decitabine, sorafenib, lestaurtinib, midostaurin, quizartinib, crenolanib, gilteritinib or cytarabine.

In an embodiment, the METTL3 inhibitor is selected any compound of the formulae I, II, VI or VII defined above, or a pharmaceutically acceptable salt thereof and the second agent is selected from daunorubicin, doxorubicin, epirubicin, idarubicin, 5-azacitidine, decitabine, sorafenib, lestaurtinib, midostaurin, quizartinib, crenolanib, gilteritinib or cytarabine.

In an embodiment, the METTL3 inhibitor is selected any compound of the formulae I, II, VI or VII defined above, or a pharmaceutically acceptable salt thereof and the second agent is selected from daunorubicin, doxorubicin, 5-azacitidine, decitabine, midostaurin, quizartinib, gilteritinib or cytarabine.

In an embodiment, the METTL3 inhibitor is selected any compound of the formulae I, II, VI or VII defined above, or a pharmaceutically acceptable salt thereof and the second agent is selected from daunorubicin, 5-azacitidine, decitabine, quizartinib, or cytarabine.

In an embodiment, the METTL3 inhibitor is selected from STM3006, STM3480 or STM3675, or a pharmaceutically-acceptable salt thereof, and the second agent is selected from daunorubicin, doxorubicin, epirubicin, idarubicin, 5-azacitidine, decitabine, sorafenib, lestaurtinib, midostaurin, quizartinib, crenolanib, gilteritinib or cytarabine.

In an embodiment, the METTL3 inhibitor is selected from STM3006, STM3480 or STM3675, or a pharmaceutically-acceptable salt thereof, and the second agent is selected from daunorubicin, doxorubicin, 5-azacitidine, decitabine, midostaurin, quizartinib, gilteritinib or cytarabine.

In an embodiment, the METTL3 inhibitor is selected from STM3006, STM3480 or STM3675, or a pharmaceutically-acceptable salt thereof, and the second agent is selected from daunorubicin, 5-azacitidine, decitabine, quizartinib, or cytarabine.

In an embodiment, the METTL3 inhibitor is STM3480, or a pharmaceutically-acceptable salt thereof, and the second agent is selected from daunorubicin, doxorubicin, epirubicin, idarubicin, 5-azacitidine, decitabine, sorafenib, lestaurtinib, midostaurin, quizartinib, crenolanib, gilteritinib or cytarabine.

In an embodiment, the METTL3 inhibitor is STM3480, or a pharmaceutically-acceptable salt thereof, and the second agent is selected from daunorubicin, doxorubicin, 5-azacitidine, decitabine, midostaurin, quizartinib, gilteritinib or cytarabine.

In an embodiment, the METTL3 inhibitor is STM3480, or a pharmaceutically-acceptable salt thereof, and the second agent is selected from daunorubicin, 5-azacitidine, decitabine, quizartinib, or cytarabine.

### Pharmaceutical Compositions

In one aspect the present invention relates to a pharmaceutical composition comprising a combination of METTL3 inhibitor as defined herein, or a pharmaceutically acceptable salt thereof, and the other agent present in the combination as defined above, or a pharmaceutically acceptable salt thereof, and one or more pharmaceutically acceptable excipients.

The pharmaceutical compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The pharmaceutical compositions of the invention will typically be for parenteral administration, especially when the inhibitors are antibodies.

The pharmaceutical compositions may be obtained by conventional procedures using conventional pharmaceutical excipients well known in the art. Thus, compositions intended for oral use may contain, for example, one or more colouring, sweetening, flavouring and/or preservative agents.

An effective amount of each component of the combination therapy will be present. Such an amount is an amount sufficient to treat or prevent a cancer condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a combination of the invention will naturally vary according to the nature and severity of the conditions, the age and sex of the animal or patient and the route of administration, according to well-known principles of medicine.

In using a combination of the invention for therapeutic or prophylactic purposes it will generally be administered within a therapeutically effective dose of the particular agent concerned. These dosages are known in the art and will vary from one agent to another. The dosage may, for example, be in the range of 0.1 mg/kg to 30 mg/kg body weight. The dosing schedule will also vary from one immune checkpoint inhibitor to another. Suitable dosing schedules are known in the art.

### Routes of Administration

The combination of the invention or pharmaceutical compositions comprising said combination may be administered to a subject by any appropriate or convenient route of administration, whether systemically/peripherally or topically (i.e., at the site of desired action).

Depending on the nature of the agent, routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Combinations with Additional Therapeutic Agents

The combination treatments defined herein may be applied as a sole therapy for the treatment of the specified condition or it may involve, in addition to the combination therapy of the present invention, one or more additional therapies (including treatment with another therapeutic agent, surgery or other therapeutic interventions such as radiotherapy in the oncology setting).

Typically, the other therapeutic agent used in combination with the combination therapy of the present invention will be one or more therapeutic agents used as the standard of care for the treatment of the disease or condition concerned. The other therapeutic agent may include, for example, another drug used for the treatment of the condition concerned, or an agent that modulates the biological response to the combination therapy of the invention, such as, for example, an immunomodulatory agent.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

For example, the combination therapies defined hereinbefore may be applied as a sole therapy or may involve, in addition to the compound of the invention, conventional surgery or radiotherapy or chemotherapy. Such chemotherapy may further include one or more of the following categories of anti-tumour agents:—
other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
vascular damaging agents such as Combretastatin A4 and compounds disclosed in International Patent Applications WO 99/02166, WO 00/40529, WO 00/41669, WO 01/92224, WO 02/04434 and WO 02/08213;
an endothelin receptor antagonist, for example zibotentan (ZD4054) or atrasentan;
antisense therapies, for example those which are directed to the targets listed above, such as ISIS 2503, an anti-ras antisense;
gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a particular embodiment, the combination therapies defined hereinbefore may involve, in addition to the combination therapy of the invention, conventional surgery or radiotherapy or chemotherapy.

Such conjoint treatment may be achieved by way of the simultaneous, sequential or separate dosing of the individual components of the treatment. Such combination products employ the combination therapy of this invention within the dosage range described hereinbefore and the other pharmaceutically-active agent within its approved dosage range.

According to this aspect of the invention there is provided a combination as defined hereinbefore for use in the treatment of a cancer as defined herein, comprising a combination therapy of the invention as defined hereinbefore, and another anti-tumour agent.

According to this aspect of the invention there is provided a combination for use in the treatment of cancer as defined herein, comprising a combination therapy of the invention as defined hereinbefore, and any one of the anti-tumour agents listed herein above.

In a further aspect of the invention there is provided a combination product of the invention for use in the treatment of cancer in combination with another anti-tumour agent, optionally selected from one listed herein above.

In a particular aspect, the present invention provides a combination of a METTL3 inhibitor and a BCL2 inhibitor as defined above for use in the treatment of acute myeloid leukaemia (AML), chronic lymphocytic leukaemia (CLL), small lymphocytic lymphoma (SLL) or myelodysplastic syndromes (MDS), wherein the METTL3 inhibitor and BCL2 inhibitor are administered in combination with an additional anti-tumour agent.

In a further aspect, the present invention provides a combination of STM3480 and venetoclax for use in the treatment of acute myeloid leukaemia (AML), chronic lymphocytic leukaemia (CLL), small lymphocytic lymphoma (SLL) or myelodysplastic syndromes (MDS), wherein STM3480 and venetoclax are administered in combination with another anti-tumour agent.

Suitably, the additional anti-tumour agent is selected from an agent that is the standard of care for the treatment of AML, CLL, SLL or MDS. In particular, the additional anti-tumour agent may be selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof.

More particularly, the additional anti-tumour agent may be selected from daunorubicin, doxorubicin, epirubicin, idarubicin, 5-azacitidine, decitabine, sorafenib, lestaurtinib, midostaurin, quizartinib, crenolanib, gilteritinib or cytarabine. Suitably, the combination is for the treatment of AML, CLL or SLL, especially AML.

In one particular aspect, the present invention provides a combination of STM3480 and venetoclax for use in the treatment of acute myeloid leukaemia (AML), wherein STM3480 and venetoclax are administered in combination with another anti-tumour agent. Suitably, the additional anti-tumour agent is selected from an agent that is the standard of care for the treatment of AML. In particular, the additional anti-tumour agent may be selected from:
(i) an anthracycline topoisomerase 2 inhibitor (e.g. daunorubicin);
(ii) cytarabine;
(iii) a hypomethylating agent (e.g. 5-azacitidine or decitabine); or
(iv) a FLT3 inhibitor (e.g. quizartinib);
   or a pharmaceutically-acceptable salt thereof. More particularly, the additional anti-tumour agent may be selected from daunorubicin, doxorubicin, epirubicin, idarubicin, 5-azacitidine, decitabine, sorafenib, lestaurtinib, midostaurin, quizartinib, crenolanib, gilteritinib or cytarabine. Suitably, the combination is for the treatment of AML, CLL or SLL, especially AML.

### DESCRIPTION OF DRAWINGS

Embodiments of the invention will be described, by way of example only, with reference to the accompanying drawings, in which:

FIG. 1—The combination of STM3480 and pembrolizumab leads to the strongest reduction in SKOV3 tumour survival (NLR intensity) and an enhanced effect relative to either agent alone.

FIG. 2—The combination of STM3480 and avelumab leads to the strongest reduction in SKOV3 tumour survival (NLR intensity) and an enhanced effect relative to either agent alone.

FIG. 3—Mean tumour volume of A20 lymphomas shown after treatments indicated, with Standard Error of the Mean indicated (SEM). Vehicle treatment (solid black lines, open circles) shows rapid progressive tumour growth. STM3480 treatment (dotted grey lines, open squares) shows a reduction of tumour growth compared to the vehicle control. Anti-PD1 treatment (grey dashed lines, open circles) shows a modest reduction in tumour growth. Combination of STM3480 and anti-PD1 (black dashed lines, closed triangles) shows complete tumour regressions in 6/10 animals. Abbreviations: TV—tumour volume; BID—twice daily dosing; BIW—twice weekly dosing.

FIG. 4—Tumour growth of A20 lymphomas shown for individual tumours after treatments indicated. Vehicle treatment (solid black lines, upper left panel) shows progressive tumour growth in 9/10 animals. STM3480 treatment (dashed grey lines, upper right panel) shows progressive tumour growth in 8/10 animals. Anti-PD1 treatment (black dotted lines, lower left panel) shows progressive tumour growth in 8/10 animals. Combination of STM3480 and anti-PD1 (black dotted and dashed lines, lower right panel) shows progressive tumour growth in 2/10 animals, tumour regressions in 8/10 and complete tumour regressions in 6/10 animals. Abbreviations: TV—tumour volume; BID—twice daily dosing; BIW—twice weekly dosing

FIG. 5—No adverse effects of treatments on body weight were observed in any group. Note decline in body weight of vehicle treated group from day 25 was a result of progression of disease and large tumour burden, and not due to the treatment per se.

FIG. 6—STM3480 shows a synergistic interaction with venetoclax in the Kasumi1 AML cell line, with increasing concentrations of STM3480 leading to decreasing IC50 of venetoclax as shown in the table below.

FIG. 7—STM3480 shows a synergistic interaction with venetoclax in the MOLM13 AML cell line, with increasing concentrations of STM3480 leading to decreasing IC50 of venetoclax as shown in the table below.

FIG. 8—STM3006 shows a synergistic interaction with venetoclax in the Kasumi1 AML cell line, with increasing concentrations of STM3006 leading to decreasing IC50 of venetoclax as shown in the table below.

FIG. 9—STM3006 shows a synergistic interaction with venetoclax in the MOLM13 AML cell line, with increasing concentrations of STM3006 leading to decreasing IC50 of venetoclax as shown in the table below.

FIG. 10—STM3675 shows a synergistic interaction with venetoclax in the Kasumi1 AML cell line, with increasing concentrations of STM3675 leading to decreasing IC50 of venetoclax as shown in the table below.

FIG. 11—STM3675 shows a synergistic interaction with venetoclax in the MOLM13 AML cell line, with increasing concentrations of STM3675 leading to decreasing IC50 of venetoclax as shown in the table below.

FIG. 12—STM3480 shows an additive interaction with daunorubicin in the Kasumi1 AML cell line, with increasing concentrations of STM3480 leading to decreasing viability. No change in IC50 of daunorubicin is observed, indicating an additive interaction.

FIG. 13—STM3006 shows an additive interaction with daunorubicin in the Kasumi1 AML cell line, with increasing concentrations of STM3006 leading to decreasing viability. No change in IC50 of daunorubicin is observed, indicating an additive interaction.

FIG. 14—STM3480 shows an additive interaction with cytarabine in the Kasumi1 AML cell line, with increasing concentrations of STM3480 leading to decreasing viability. No change in IC50 of cytarabine is observed, indicating an additive interaction.

FIG. 15—STM3006 shows an additive interaction with cytarabine in the Kasumi1 AML cell line, with increasing concentrations of STM3006 leading to decreasing viability. No change in IC50 of cytarabine is observed, indicating an additive interaction.

FIG. 16—STM3480 shows an additive interaction with 5′-azacitidine in the Kasumi1 AML cell line, with increasing concentrations of STM3480 leading to decreasing viability. No change in IC50 of 5′-azacitidine is observed, indicating an additive interaction.

FIG. 17—STM3006 shows an additive interaction with 5′-azacitidine in the Kasumi1 AML cell line, with increasing concentrations of STM3006 leading to decreasing viability. No change in IC50 of 5′-azacitidine is observed, indicating an additive interaction.

FIG. 18—STM3480 shows an additive interaction with quizartinib in the FLT3-mutant MOLM-14 AML cell line, with increasing concentrations of STM3480 leading to decreasing viability. No change in IC50 of quizartinib is observed, indicating an additive interaction.

FIG. 19—STM3006 shows an additive interaction with quizartinib in the FLT3-mutant MOLM-14 AML cell line, with increasing concentrations of STM3006 leading to decreasing viability. No change in IC50 of quizartinib is observed, indicating an additive interaction.

### EXAMPLES

### Preparation of Compounds STM3480, STM3675 and STM3006

The following abbreviations have been used:
AIBN—Azobisisobutyronitrile
DBU—1,8-Diazabicyclo[5.4.0]undec-7-ene
DCE—Dichloroethane
DCM—Dichloromethane
DIBAL—Diisobutylaluminium hydride
DIPEA—N-ethyl-N-isopropyl-propan-2-amine
DMAP—4-Dimethylaminopyridine
DMF—Dimethylformamide
DMSO—Dimethyl sulfoxide
DPPA—Diphenylphosphoryl azide
HATU—[dimethylamino(triazolo[4,5-b]pyridin-3-yloxy)methylene]-dimethyl-ammonium; hexafluorophosphate
HPLC—High performance liquid chromatography
IPA—Isopropanol
LCMS—Liquid chromatograph mass spectrometry
NBS—N-Bromosuccinimide
NMP—N-Methyl-2-pyrrolidone
Phase separation cartridge—Telos phase separator 6 mL
RBF—Round bottomed flask
RM—Reaction mixture
RT—Retention Time
STAB—Sodium triacetoxyborohydride
T3P—Propylphosphonic anhydride
TBAF—Tetra-n-butylammonium fluoride
TEA—Triethylamine
TFA—Trifluoroacetic acid
TFAA—Trifluoroacetic anhydride
THF—Tetrahydrofuran
   The Following Methodologies have been Used in the Examples:
LCMS method A refers to low pH analysis using a mobile phase consisting of 0.1% formic acid in a gradient of 5-100% MeCN in water over 1.2 min at a flow rate of 1.2 mL/min. The stationary phase consisted of a Kinetex Core-Shell C18, 2.1 mm×50 mm, 5 μm. The experiment was run at 40° C.
LCMS Method B refers to high pH analysis using a mobile phase consisting of 2 mM ammonium bicarbonate, buffered to pH10 in a gradient of 5-100% MeCN in water over 2.1 min at a flow rate of 1.0 mL/min. The stationary phase consisted of a Phenomenex Gemini-NX C18, 2.0×50 mm, 3 μm. The experiment was run at 40° C.
LCMS Method C refers to high pH analysis using a mobile phase consisting of 2 mM ammonium bicarbonate, buffered to pH10 in a gradient of 5-100% MeCN in water over 5.8 min at a flow rate of 0.6 mL/min. The stationary phase consisted of a Waters UPLC® BEH™ C18, 2.1×100 mm, 1.7 μm. The experiment was run at 40° C.

### Intermediate 1: 4-Oxopyrido[1,2-a]pyrimidine-2-carboxylic Acid

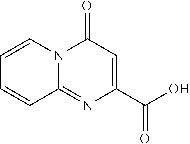 [SMILES: O=C(O)c1cc(=O)n2ccccc2n1]

Methyl 4-oxopyrido[1,2-a]pyrimidine-2-carboxylate [Tetrahedron (2014), 70(17), 2761-2765] (3.94 g, 19.3 mmol) was dissolved in hydrogen chloride solution (8M, 7.5 mL) at room temperature (An exotherm was noted on addition) and the mixture was heated at reflux at for 2 h. The mixture was cooled to room temperature and the precipitate was collected by filtration and dried under vacuum to give the title compound (3.00 g, 81%) as a white solid.

Method A: LC-MS (electrospray): m/z=191.1 (M+H)⁺, RT=0.32 min

### Preparation of STM3480—N-[(2-{[(cyclobutylmethyl)amino]methyl}-1H-indol-6-yl)methyl]-4-oxo-4H-pyrido[1,2-a]pyrimidine-2-carboxamide

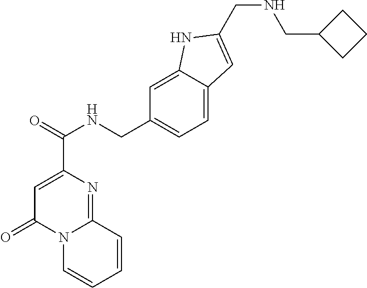 [SMILES: O=C(NCc1ccc2cc(CNCC3CCC3)[nH]c2c1)c1cc(=O)n2ccccc2n1]

### Step 1: 3-amino-4-(3,3-diethoxyprop-1-yn-1-yl)benzonitrile

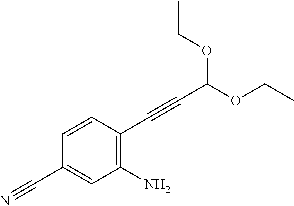 [SMILES: CCOC(C#Cc1ccc(C#N)cc1N)OCC]

To a solution of 3-amino-4-iodobenzonitrile (8.00 g, 32.8 mmol) in THF-Anhydrous (40 mL) and triethylamine (80 mL, 0.574 mol) was added Pd₂(PPh₃)₂Cl₂ (230 mg, 0.328 mmol) and triphenylphosphine (172 mg, 0.656 mmol) at ambient temperature. The solution was degassed by bubbling nitrogen into the solution for 20 minutes. Then copper (I) iodide (125 mg, 0.66 mmol) and 3,3-diethoxyprop-1-yne (5.04 g, 39.3 mmol) were added sequentially and the reaction was stirred under nitrogen atmosphere for 18 hours. The precipitate (triethylamine hydrochloride) was collected by filtration and washed with EtOAc (˜20 mL). The filtrate was concentrated at reduced pressure and the residue was purified by chromatography on SiO₂ [Biotage KP-Sil 100 g, eluting with 0-50% EtOAc in heptane]. The product containing fractions were combined and concentrated in vacuo to afford the title compound (8.19 g, Quant) as an orange oil.

Method B: LC-MS (electrospray): m/z=262.3 (M+H)⁺, RT=1.65 min.

### Step 2: 2-(diethoxymethyl)-1H-indole-6-carbonitrile

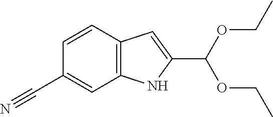 [SMILES: CCOC(OCC)c1cc2ccc(C#N)cc2[nH]1]

To a stirred solution of 3-amino-4-(3,3-diethoxyprop-1-ynyl)benzonitrile (8.00 g, 31.1 mmol) in NMP (99 mL) was added potassium tert-butoxide (6.98 g, 62.2 mmol) at 0° C. (the colour of the solution turned from orange to dark red). After warming to RT, the solution was stirred at ambient temperature for 16 hours. Saturated aqueous ammonium chloride solution (25 mL) was added and the resulting mixture was partitioned between EtOAc (250 mL) and water (250 mL). The layers were separated and the organic layer washed twice more with water (2×200 mL). The organic layer was dried over sodium sulfate, filtered and concentrated in vacuo to give a brown oil. The first aqueous layer was re-extracted with EtOAc (200 mL) and the layers separated. The organic layer was washed twice with water (2×200 mL). The organic layer was dried over sodium sulfate, filtered and concentrated in vacuo to give an orange oil. The crude material was purified by chromatography on SiO₂ [BIOTAGE KP-Sil 100 g, eluting with 0-50% EtOAc in heptane]. The product containing fractions were combined and concentrated in vacuo. The residue (yellow solid) was recrystallised from EtOAc/heptane to afford the title compound (5.86 g, 24.0 mmol, 77%) as colourless crystalline solid. Method B: LC-MS (electrospray): m/z=262.3 (M+H)⁺, RT=1.69 min.

### Step 3: [2-(diethoxymethyl)-1H-indol-6-yl]methanamine

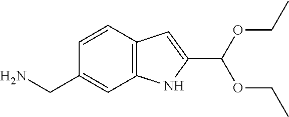 [SMILES: CCOC(OCC)c1cc2ccc(CN)cc2[nH]1]

To a degassed solution of 2-(diethoxymethyl)-1H-indole-6-carbonitrile (5.8 g, 24 mmol) in ethanol (70 mL) was added ammonia in MeOH (7M, 20 mL, 0.14 mmol) and the reaction was degassed and backfilled with nitrogen 3 times. Raney nickel (assumed 50%, about 5.4 g, 0.1 mmoL) was added and the reaction evacuated and backfilled with nitrogen 3 times. The flask was evacuated one final time and put under a hydrogen atmosphere and stirred at ambient temperature for 3 hours. More Raney nickel (about 2.7 g) was added and the reaction evacuated and placed under a hydrogen atmosphere and stirred at ambient temperature for 16 hours. The catalyst was removed by filtration (through Kieselguhr) and washed with methanol (50 mL). The filtrate was concentrated under reduced pressure to afford the title compound (5.96 g, 100%) as a colourless oil which crystallised upon standing.

Method C: LC-MS (electrospray): m/z=247.3 (M−H)⁻, RT=2.74 min.

### Step 4: N-[(2-formyl-1H-indol-6-yl)methyl]-4-oxo-pyrido[1,2-a]pyrimidine-2-carboxamide

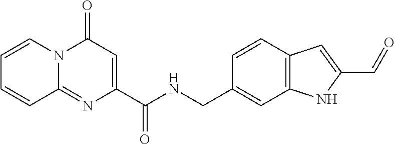 [SMILES: O=Cc1cc2ccc(CNC(=O)c3cc(=O)n4ccccc4n3)cc2[nH]1]

To a stirred solution of 4-oxopyrido[1,2-a]pyrimidine-2-carboxylic acid (455 mg, 2.39 mmol) (Intermediate 1) and DIPEA (1.0 mL, 5.98 mmol) in DMF (10 mL) was added HATU (910 mg, 2.39 mmol). A colour change was observed from colourless to green and a suspension formed. After a further 30 minutes of stirring at ambient temperature, a solution of [2-(diethoxymethyl)-1H-indol-6-yl]methanamine (500 mg, 1.99 mmol) in DMF (5 mL) was added dropwise to the reaction. A colour change of green to red was observed and the reaction became homogeneous and was stirred at ambient temperature overnight.

The mixture was partitioned between EtOAc (100 mL) and sat. NaHCO₃ solution (50 mL). The organic layer was separated, washed with water (80 mL) and brine (20 mL), dried (Na₂SO₄), filtered and concentrated at reduced pressure to give a viscous red oil.

The crude product was dissolved in THF (10 mL), water (1 mL) and acetic acid (0.5 mL) were added and the mixture was stirred at ambient temperature for 2 hours.

The THF was removed in vacuo and water (10 mL) was added to the resulting mixture causing further solid to precipitate out. The brown solid was collected by washed with water (2×5 mL) then ether (3×5 mL) and dried under vacuum to give the title compound (520 mg, 75%) as a brown solid.

Method C: LC-MS (electrospray): m/z=347.2 (M+H)⁺, RT=2.37 min.

### Step 5: N-[[2-[(Cyclobutylmethylamino)methyl]-1H-indol-6-yl]methyl]-4-oxo-pyrido[1,2-a]pyrimidine-2-carboxamide

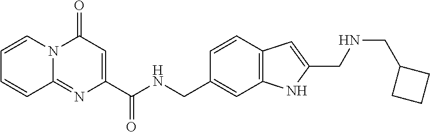 [SMILES: O=C(NCc1ccc2cc(CNCC3CCC3)[nH]c2c1)c1cc(=O)n2ccccc2n1]

A pressure vial was charged with N-[(2-formyl-1H-indol-6-yl)methyl]-4-oxo-pyrido[1,2-a]pyrimidine-2-carboxamide (185 mg, 0.53 mmol), DCE (5 mL) and 1-cyclobutylmethanamine (0.13 mL, 1.0 mmol) at ambient temperature. The vial was sealed and the mixture was stirred at 65° C. for 2 hours. After cooling to RT, sodium triacetoxyborohydride (340 mg, 1.85 mmol) was added and the mixture was heated to 65° C. for 2 hours.

The mixture was partitioned between EtOAc (40 mL) and sat. sodium bicarbonate solution (30 mL). The organic layer was separated, washed with brine (20 mL), dried (Na₂SO₄), filtered and concentrated at reduced pressure. The residue (pale yellow oil) was purified by reverse phase chromatography (basic method, SNAP ULTRA 30 g Cartridge, eluting with MeCN+0.1% NH₃/H₂O+0.1% NH₃, 10 to 90%). The fractions containing desired product were freeze dried overnight to give the title compound (85 mg, 38%) as an off-white solid.

Method C: LC-MS (electrospray): m/z=416.4 (M+H)⁺, RT=3.14 min.

### Preparation of STM3675—N-[(2-{[({3-fluorobicyclo[1.1.1]pentan-1-yl}methyl)amino]methyl}-1H-indol-6-yl)methyl]-4-oxo-4H-pyrido[1,2-a]pyrimidine-2-carboxamide

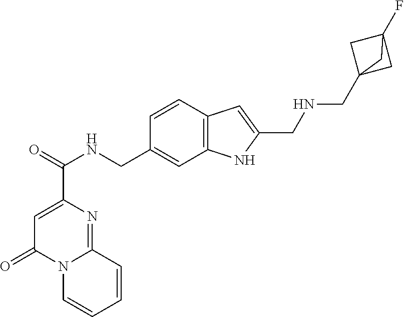 [SMILES: O=C(NCc1ccc2cc(CNCC34CC(F)(C3)C4)[nH]c2c1)c1cc(=O)n2ccccc2n1]

This compound was prepared in the same manner as STM3480 described above using commercial amines or described intermediates.

Method C: LC-MS (electrospray): m/z=446.4 (M+H)⁺, RT=3.01 min.

### Preparation of STM3006—(6-bromo-4-[1-({6-[(4,4-dimethylpiperidin-1-yl)methyl]imidazo[1,2-a]pyridin-2-yl}methyl)-1H-1,2,3-triazol-4-yl]-1H-indazole)

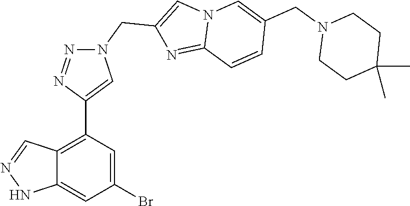 [SMILES: CC1(C)CCN(Cc2ccc3nc(Cn4cc(-c5cc(Br)cc6[nH]ncc56)nn4)cn3c2)CC1]

The preparation of STM3006 is described in Example 227 on page 285 of International Patent Publication No. WO2020/050898.

### Example 1—Combinations of METTL3 Inhibitors (STM3480) with Immune Checkpoint Inhibitors

### Tumour Killing Assay (TKA)

This experiment evaluates the effects of human donor Peripheral Blood Mononuclear Cells (PBMCs) to attack and kill fluorescently-labelled tumour cells in a co-culture assay. Briefly, test agents are incubated at different concentrations with SKOV3 human ovarian cancer cells in the presence or absence of human donor PBMCs and tumour growth is monitored continuously by real time imaging of the NucLight Red-labelled SKOV3 cells for several days.

### Tumour Killing Assay Method

NucLight Red (Incucyte, Essen Biosciences)-transduced SK-OV-3 (SK-OV-3-NLR) cells were transferred into Ultra-low attachment 96 well plates with spontaneous spheroid formation occurring over 48 hours. Peripheral blood mononuclear cells (PBMC) were isolated from healthy single donor buffy coats by density separation. Prior to co-culture of SK-OV-3-NLR spheroids with PBMC, SK-OV-3-NLR spheroids were treated with 10 ng/ml of interferon gamma (IFNγ). All treatments, including STM3480 and Pembrolizumab, were added at the point of co-culture in a final total volume of 200 μl/per well. Assay plates were transferred into an IncuCyte S3 and images were collected every 2 hours for 7 days. Following experimental completion, an analysis definition was applied to quantify the Total NLR Integrated Intensity present on a per well basis. Changes in spheroid size were quantified by plotting Total NLR Integrated Intensity against time.

TABLE 1

| | Final concentration | | |
|---|---|---|---|
| Condition | in assay | Cells | Readout |
| Untreated | N/A | SK-OV-3 | Total NLR |
| DMSO (vehicle) | 0.06% | spheroids +/− | Integrated |
| Pembrolizumab | 20 μg/mL | PBMC | Intensity |
| IgG4 isotype | 20 μg/mL | | |
| control | | | |
| STM3480 | 0.5, 0.1 & 0.05 μM | | |
| STM3480 + | 0.5, 0.1 & 0.05 μM | | |
| Pembrolizumab | (STM3480) + 20 μg/ml | | |
| | (Pembrolizumab) | | |

### Results

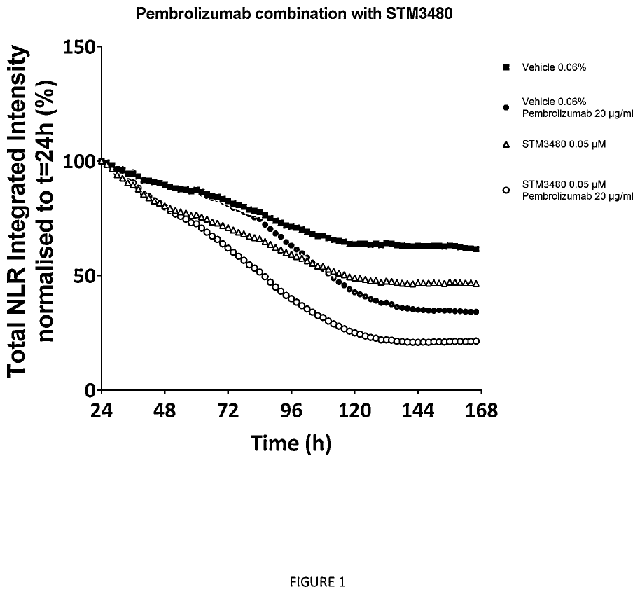

FIG. 1 shows the changes of tumour viability (normalized NucLight Red [NLR] fluorescence on the y axis) against time (hours in the x axis). Vehicle-treated cells (closed black squares) show a steady modest decline in fluorescence intensity with time. Pembrolizumab (anti-PD1 antibody)-treated cells (closed grey circles) shows a greater decline in viable tumour cells at the end of the study. 0.05 micromolar STM3480 treatment (open triangles) yields a greater anti-tumour effect compared to vehicle, whereas the combination of STM3480 and pembrolizumab (open circles) gives the greatest reduction in tumour cell viability, beyond the effects seen with the individual agents.

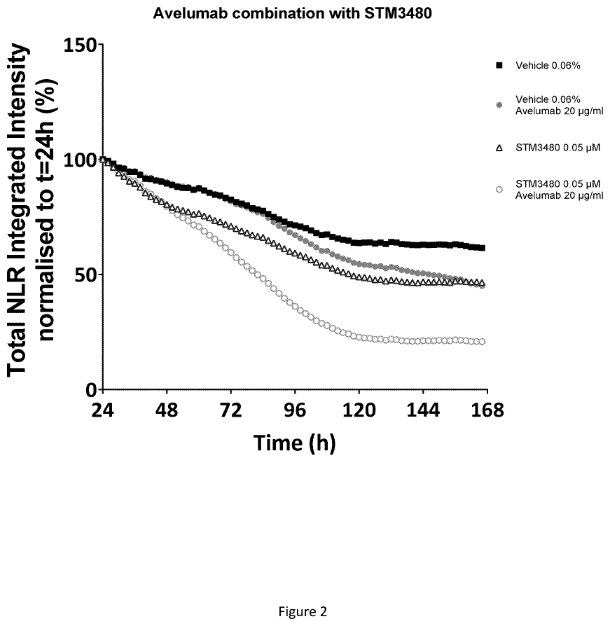

Similarly, the treatment of SKOV3/PBMC co-cultures with STM3480 and/or avelumab (anti-PD-L1 antibody; open circles) leads to the greatest anti-tumour activity, greater than that seen with either single agent alone (grey circles, open triangles), demonstrating the surprising and beneficial effect of this combination treatment (see FIG. 2).

### Example 2—Combinations of METTL3 Inhibitors (STM3480) with Immune Checkpoint Inhibitors—In Vivo Studies

The in vivo effect of METTL3 inhibition in combination with anti-PD1 immune checkpoint inhibitor was investigated using syngeneic mouse tumour models in immune-competent hosts. Mouse tumour models can be grown in appropriate host strains and are widely used to investigate the effects of therapeutic agents on the immune system and cancer growth. There are a limited range of mouse syngeneic tumour models available, far fewer than human cell lines, and they are widely used in the art to support potential clinical utility of immune stimulatory approaches across multiple indications. We exemplify the potential role of STM3480 and combinations with immune checkpoint inhibitors such as anti-PD-1 antibodies in multiple tumour models including A₂₀ (murine B cell lymphoma), EMT-6 (murine breast cancer) and CT-26 (murine colorectal cancer).

### 1. A20 Lymphoma Model

### Protocol

The objective of this study is to assess the anti-tumour efficacy of STM3480 in combination with anti-PD1 in subcutaneous A20 mouse B cell lymphoma cancer model in female BALB/c mice.

A20 (#ATCC TIB-208) mouse B cell lymphoma cells were cultured in (RPMI-1640+10% non H.I. FBS+0.05 mM 2-mercaptoethanol) media. 5×10⁵ viable cells in 0.1 ml PBS were injected subcutaneously into the left flank of 7-9 week old female BALB/cN (BALB/cAnNCrl) Crl mice utilising 27 gauge needles following skin disinfection with 70% ethanol.

Tumours were measured three times a week and tumour volumes estimated using the formula 0.5 (L×W²) by measuring the tumour in two dimensions using electronic callipers for the duration of the study.

Mice were randomly allocated to four treatment groups and treatment commenced when tumours reached a mean volume of ˜50-100 mm³. Mice were allocated to their treatment groups with uniform mean tumour volume between groups. Treatment was continued for up to 3 weeks, or until individual mice were sacrificed for humane reasons according to Home Office license regulations (e.g. body weight loss >20%; tumour volume >1500 mm³; tumour ulceration; loss of clinical condition). The final dose was given on day 32 after implantation.

### Formulation

Vehicle for STM3480—Hydroxypropyl-β-Cyclodextrin (HPBCD)/Sodium Acetate buffer pH:4.6 50 mM (10%/90%; w/v): Weigh 1.025 g of sodium acetate anhydrous. Add 700 μl of glacial acetic acid then make up to 500.0 ml with ultrapurified water. pH:4.6 50 mM.

For 1 ml volume 5 mg/ml STM3480 dosing solution: Weigh 5.0 mg of STM00003480 compound (Batch: EV-WZM001-421-002). Add 1.0 mL of HPBCD/Acetate buffer pH:4.6 50 mM (10%/90%; w/v). Magnetic stirring at high speed at ambient temperature; Overnight stirring may be required if there are any signs of precipitation. The formulation is a slight yellow solution with very few particles in suspension. Homogenize with a vortex before administration.

For PD-1 treatments, a mouse-specific anti-PD1 antibody (clone ID RMP1-14) was used, dissolved in PBS as described in the table below.

TABLE 2

| Group | | | Dose | Dose | | |
|---|---|---|---|---|---|---|
| (No per | | Dose | Volume | Concentration | Route/ | Dosing |
| group) | Compound ID | (mg/kg) | (ml/kg) | (mg/ml) | N/A | Frequency |
| 1 (10) | Vehicle | 0 | 10 | 0 | p.o | BID × up to |
| | (HPBCD + Acetate) | | | | | 3 weeks |
| 2 (10) | STM3480 | 50 | \hat{ }10 | 5 | p.o | QD × up to |
| | | | | | | 3 weeks |
| 3 (10) | Anti PD1 | 10 | 10 | 1 | i.p. | BIW × up |
| | | | | | | to 3 weeks |
| 4 (10) | STM3480 | 50 | \hat{ }10 | 5 | p.o | QD × up to |
| | | | | | | 3 weeks |
| | Anti PD1 | 10 | 10 | 1 | i.p. | BIW × up |
| | | | | | | to 3 weeks |

p.o. = oral route (by gavage)-maximum volume 20 mL/kg
i.p. = Intraperitoneal-maximum volume 20 ml/kg
BID =~8 h apart
\hat{ }For STM3480 the total dosing volume is 20 ml/kg per day so it was 10 ml/kg per dose for BID dosing
Animal welfare for this study complies with the UK Animals Scientific Procedures Act 1986 (ASPA) in line with Directive 2010/63/EU of the European Parliament and the Council of 22 Sep. 2010 on the protection of animals used for scientific purposes.

### Data

Vehicle treatment (solid black lines, upper left panel) shows progressive tumour growth in 9/10 animals. STM3480 treatment (dashed grey lines, upper right panel) shows progressive tumour growth in 8/10 animals. Anti-PD1 treatment (black dotted lines, lower left panel) shows progressive tumour growth in 8/10 animals. Combination of STM3480 and anti-PD1 (black dotted and dashed lines, lower right panel) shows progressive tumour growth in 2/10 animals, tumour regressions in 8/10 and complete tumour regressions in 6/10 animals.

Animals treated with the combination of STM3480 and anti-PD1 antibodies showed complete tumour regressions (i.e. no measurable tumour remaining) in the majority of treated animals. 6/10 animals remained tumour-free for 29 days after treatment ceased, indicating that these animals had all been cured.

### Results

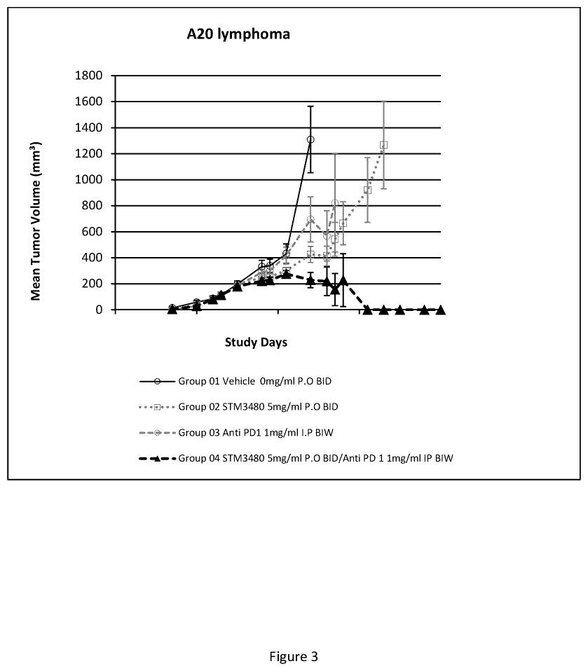

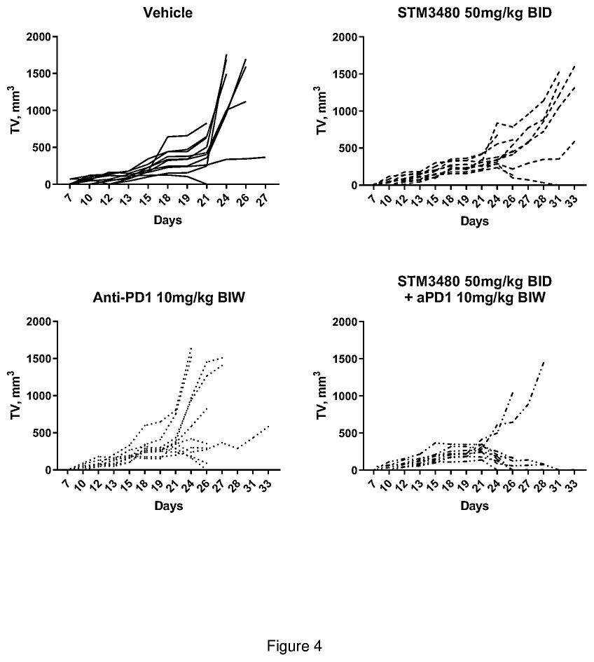

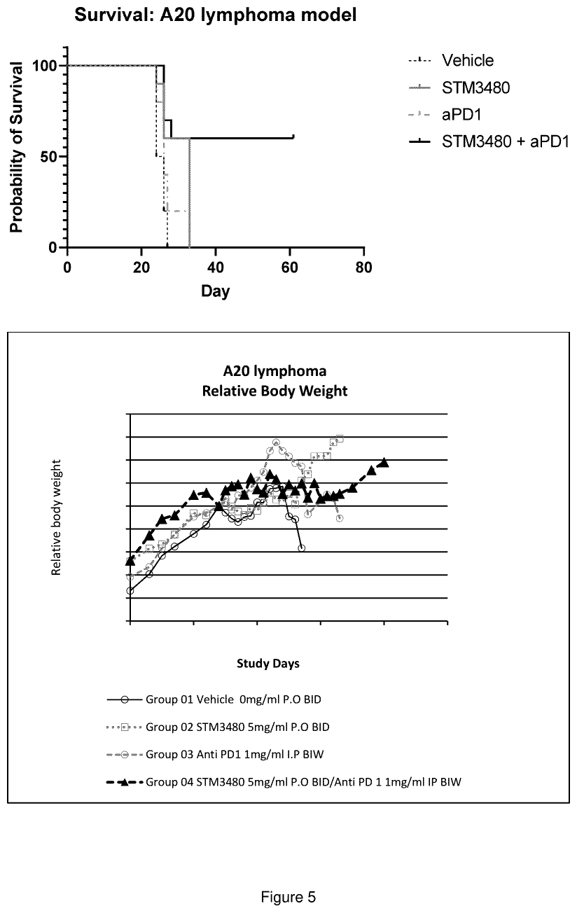

See Table 5 below and FIG. 3 [A20 B cell lymphoma in vivo model (mean tumour volume)]; FIG. 4 [A20 B cell lymphoma in vivo model (individual tumour curves)] and FIG. 5 [A20 B cell lymphoma in vivo model (survival)].

### 2. EMT6 Breast Cancer Model

### Protocol

The objective of this study is to assess the anti-tumour efficacy of STM3480 in combination with anti-PD1 in subcutaneous EMT6 mouse breast cancer model in female BALB/c mice.

EMT6 (#ATCC CRL-2755) mouse breast cancer cells were cultured in (Waymouth's MB 752/1 Medium with 2 mM L-glutamine, 85%; foetal bovine serum (non-heat inactivated), 15%) media. 5×10⁴ viable cells in 0.1 ml PBS were injected subcutaneously into the left flank of 8-9 week old female BALB/cN (BALB/cAnNCrl) Crl mice utilising 27 gauge needles following skin disinfection with 70% ethanol.

Tumours were measured three times a week and tumour volumes estimated using the formula 0.5 (L×W²) by measuring the tumour in two dimensions using electronic callipers for the duration of the study.

Mice were randomly allocated to four treatment groups and treatment commenced when tumours reached a mean volume of ˜50-100 mm³. Mice were allocated to their treatment groups with uniform mean tumour volume between groups. Treatment was continued for up to 3 weeks, or until individual mice were sacrificed for humane reasons according to Home Office license regulations (e.g. body weight loss >20%; tumour volume >1500 mm³; tumour ulceration; loss of clinical condition). The final dose was given on day 32 after implantation.

TABLE 3

| Group | | | Dose | Dose | | |
|---|---|---|---|---|---|---|
| (No per | | Dose | Volume | Concentration | Route/ | Dosing |
| group) | Compound ID | (mg/kg) | (ml/kg) | (mg/ml) | N/A | Frequency |
| 1 (10) | Vehicle | 0 | 10 | 0 | p.o | BID × up to |
| | (HPBCD + Acetate) | | | | | 3 weeks |
| 2 (10) | STM3480 | 50 | \hat{ }10 | 5 | p.o | QD × up to |
| | | | | | | 3 weeks |
| 3 (10) | Anti PD1 | 10 | 10 | 1 | i.p. | BIW × up |
| | | | | | | to 3 weeks |
| 4 (10) | STM3480 | 50 | \hat{ }10 | 5 | p.o | QD × up to |
| | | | | | | 3 weeks |
| | Anti PD1 | 10 | 10 | 1 | i.p. | BIW × up |
| | | | | | | to 3 weeks |

p.o. = oral route (by gavage)-maximum volume 20 mL/kg
i.p. = Intraperitoneal-maximum volume 20 mL/kg
BID =~8 h apart
\hat{ }For STM3480 the total dosing volume is 20 ml/kg per day so it was 10 ml/kg per dose for BID dosing
Animal welfare for this study complies with the UK Animals Scientific Procedures Act 1986 (ASPA) in line with Directive 2010/63/EU of the European Parliament and the Council of 22 Sep. 2010 on the protection of animals used for scientific purposes.

### Results

See Table 5 below.

### 3. CT26 Colorectal Cancer Model

### Protocol

The objective of this study is to assess the anti-tumour efficacy of STM3480 in combination with anti-PD1 in subcutaneous CT26 mouse colorectal cancer model in female BALB/c mice. CT26.WT (CRL-2638) mouse breast cancer cells were cultured in (RPMI—1640+10% FBS+2 mM L-Glutamine) media. 1×10⁵ viable cells in 0.1 ml PBS were injected subcutaneously into the left flank of 8-9 week old female BALB/cN (BALB/cAnNCrl) Crl mice utilising 27 gauge needles following skin disinfection with 70% ethanol.

Tumours were measured three times a week and tumour volumes estimated using the formula 0.5 (L×W²) by measuring the tumour in two dimensions using electronic callipers for the duration of the study.

Mice were randomly allocated to four treatment groups and treatment commenced when tumours reached a mean volume of ˜50-100 mm³. Mice were allocated to their treatment groups with uniform mean tumour volume between groups. Treatment was continued for up to 3 weeks, or until individual mice were sacrificed for humane reasons according to Home Office license regulations (e.g. body weight loss >20%; tumour volume >1500 mm³; tumour ulceration; loss of clinical condition). The final dose was given on day 32 after implantation.

TABLE 4

| Group | | | Dose | Dose | | |
|---|---|---|---|---|---|---|
| (No per | | Dose | Volume | Concentration | Route/ | Dosing |
| group) | Compound ID | (mg/kg) | (ml/kg) | (mg/ml) | N/A | Frequency |
| 1 (10) | Vehicle | 0 | 10 | 0 | p.o | BID × up to |
| | (HPBCD + Acetate) | | | | | 3 weeks |
| 2 (10) | STM3480 | 50 | \hat{ }10 | 5 | p.o | QD × up to |
| | | | | | | 3 weeks |
| 3 (10) | Anti PD1 | 10 | 10 | 1 | i.p. | BIW × up |
| | | | | | | to 3 weeks |
| 4 (10) | STM3480 | 50 | \hat{ }10 | 5 | p.o | QD × up to |
| | | | | | | 3 weeks |
| | Anti PD1 | 10 | 10 | 1 | i.p. | BIW × up |
| | | | | | | to 3 weeks |

p.o. = oral route (by gavage)-maximum volume 20 mL/kg
i.p. = Intraperitoneal-maximum volume 20 mL/kg
BID = ~8 h apart
\hat{ }For STM3480 the total dosing volume is 20 ml/kg per day so it was 10 ml/kg per dose for BID dosing
Animal welfare for this study complies with the UK Animals Scientific Procedures Act 1986 (ASPA) in line with Directive 2010/63/EU of the European Parliament and the Council of 22 Sep. 2010 on the protection of animals used for scientific purposes.

### Results

See Table 5 below.

TABLE 5
summary of A20, EMT6, CT26 efficacy

| Tumour | | | | | | STM3480 + |
|---|---|---|---|---|---|---|
| model | Indication | | Vehicle | STM3480 | aPD1 | aPD1 |
| A20 | Lymphoma | Mean tumour | 1309 | 425 | 694 | 229 |
| | | volume d24 | | | | |
| | | (mm3) | | | | |
| | | No. mice with | 1 | 1 | 1 | 6 |
| | | complete | | | | |
| | | regressions | | | | |
| EMT6 | Breast | Mean tumour | 234 | 141 | 104 | 105 |
| | | volume d21 | | | | |
| | | (mm3) | | | | |
| | | No. mice with | 0 | 1 | 2 | 4 |
| | | complete | | | | |
| | | regressions | | | | |
| CT26 | Colorectal | Mean tumour | 1086 | 768 | 1095 | 641 |
| | | volume d28 | | | | |
| | | (mm3) | | | | |
| | | No. mice with | 0 | 0 | 0 | 2 |
| | | regressions | | | | |

The combination of STM3480 and anti-PD-1 antibody was highly efficacious in multiple in vivo syngeneic cancer models derived from a range of cancer indications (Table). In each case, the combination treatment caused the greatest number of tumour regressions compared to single agents, and in two models (A20 and EMT6), these regressions were complete and no tumour was detected after treatment, indicating a profound anti-tumour response.

### Example 3—Combinations of METTL3 Inhibitors (STM3480/STM3006/STM3675) with Venetoclax

The sensitivity of AML cell lines to combinations of METTL3 inhibitor and standards of care was determined as described. Dose response curves of venetoclax were determined either alone or in the presence of different concentrations of METTL3 inhibitor that are known to be active in cells.

Synergistic interactions occur when the IC50 value decreases in the presence of METTL3 inhibitor. Additive interactions occur when the IC50 value does not change in the presence of METTL3 inhibitor. Antagonistic interactions occur when the IC50 value increases in the presence of METTL3 inhibitor.

### Results

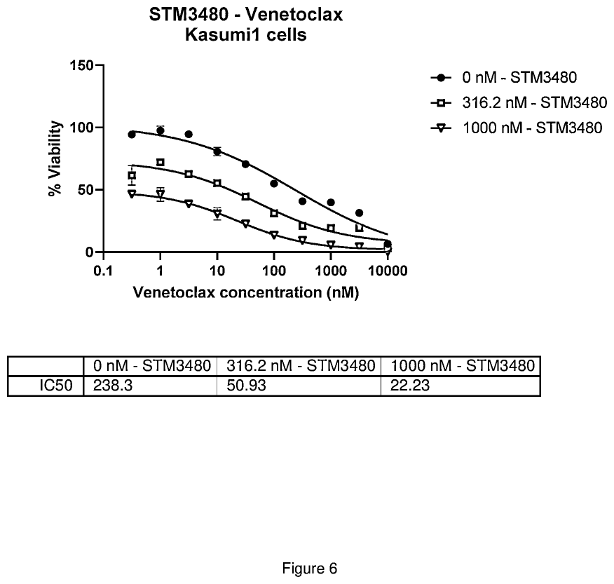

See FIGS. 6 to 11.

FIG. 6—STM3480 shows a synergistic interaction with venetoclax in the Kasumi1 AML cell line, with increasing concentrations of STM3480 leading to decreasing IC50 of venetoclax as shown in the table below.

In FIG. 6, the IC50 of venetoclax in Kasumi1 cells was 238.5 nM. In the presence of 316.2 nM STM3480, the venetoclax IC50 dropped to 50.93 nM, and in the presence of 1000 nM STM3480, the venetoclax IC50 dropped further to 22.23 nM. This increase potency indicates a synergistic interaction.

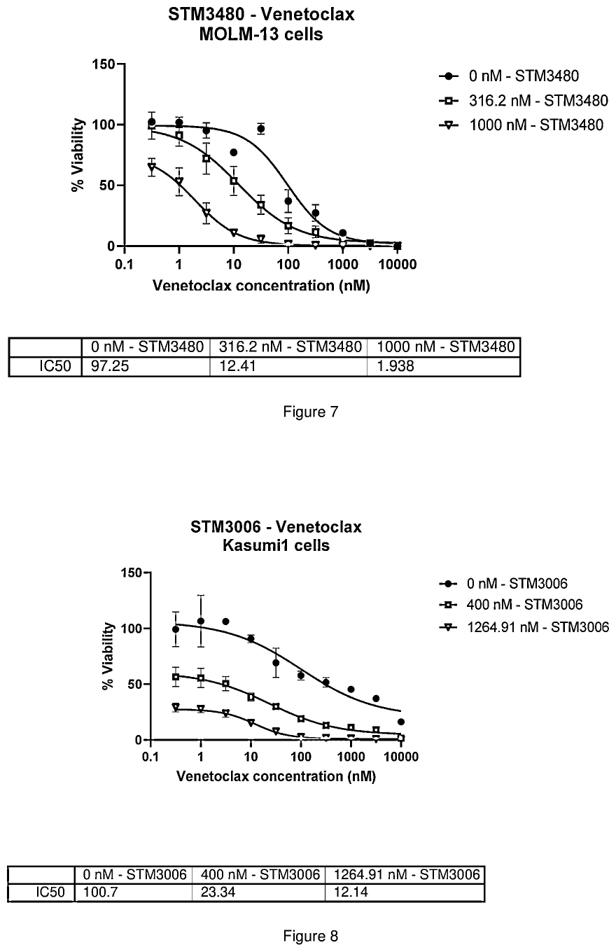

The same synergistic interaction and decrease in IC50 values were observed in the Kasumi1 cell line with additional METTL3 inhibitors, and in other AML cell lines (see FIGS. 7 to 11).

FIG. 7—STM3480 shows a synergistic interaction with venetoclax in the MOLM13 AML cell line, with increasing concentrations of STM3480 leading to decreasing IC50 of venetoclax as shown in the table below.

FIG. 8—STM3006 shows a synergistic interaction with venetoclax in the Kasumi1 AML cell line, with increasing concentrations of STM3006 leading to decreasing IC50 of venetoclax as shown in the table below.

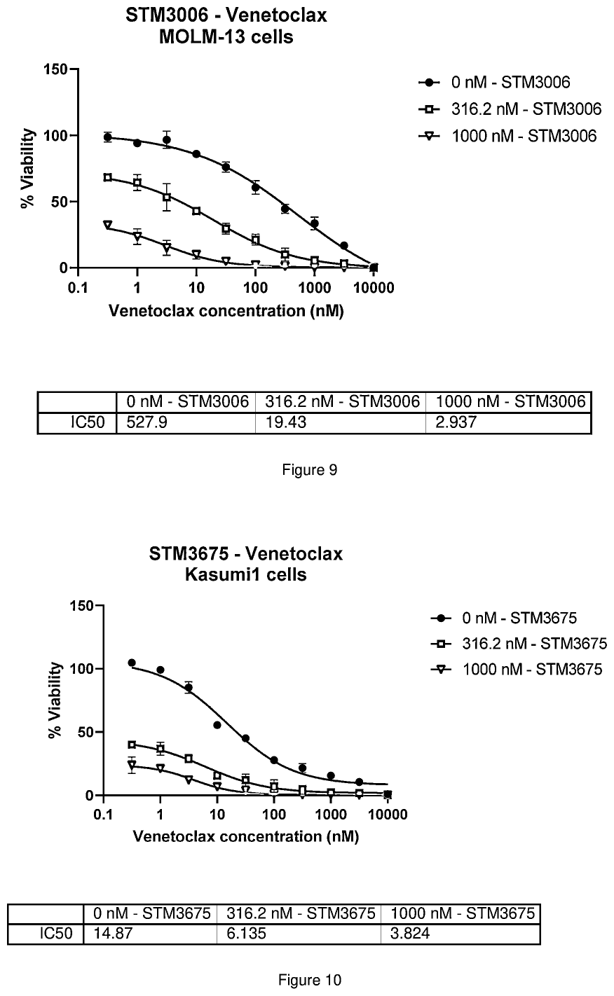

FIG. 9—STM3006 shows a synergistic interaction with venetoclax in the MOLM13 AML cell line, with increasing concentrations of STM3006 leading to decreasing IC50 of venetoclax as shown in the table below.

FIG. 10—STM3675 shows a synergistic interaction with venetoclax in the Kasumi1 AML cell line, with increasing concentrations of STM3675 leading to decreasing IC50 of venetoclax as shown in the table below.

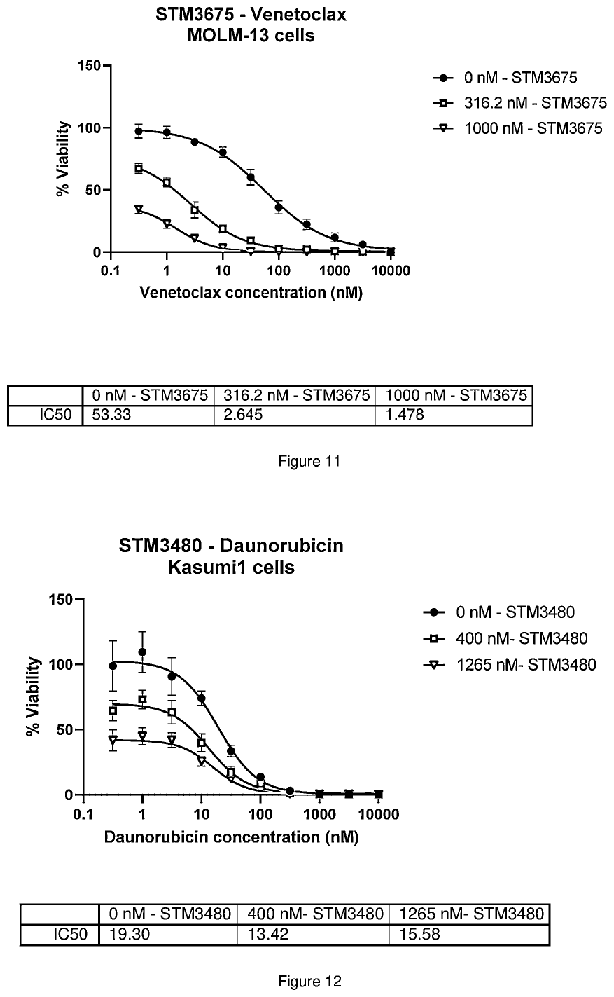

FIG. 11—STM3675 shows a synergistic interaction with venetoclax in the MOLM13 AML cell line, with increasing concentrations of STM3675 leading to decreasing IC50 of venetoclax as shown in the table below.

### Example 4 Combinations with Other AML Standards of Care Drugs

### Materials

RPMI 1640 Medium, no glutamine (Gibco #31870-025)
Fetal Bovine Serum (Sigma F7524-500ML)
GlutaMAX™ Supplement (Gibco #35050038)
Sodium Pyruvate (100 mM) (Gibco #11360039)
DMSO (Sigma #D2650)
DPBS 1× (Gibco #14190-094)
IFN gamma (R/D Systems, ref 285-IF) (200 g/ml)
Cell culture 384 well plate SBIO ultra low attachment (SBio, MS-9384WZ)
Grip Tips Viaflo 384, sterile (Integra 6464)
CellTiter-Glo Luminescent cell viability assay (Promega #G7571)
T8+ dispensehead cassettes HP (Fisher #15429293)
D4+ dispensehead cassettes HP (Fisher #15577409)

TABLE 6
Cell lines:

| | | | Treat- |
|---|---|---|---|
| | | | ment |
| Cell line | Culture medium | Cell seeding | time |
| Kasumi-1 | RPMI 1640 media 20% FBS, | 333 000 cells/ml | 3 days |
| | Glutamax and Sodium | 250 000 cells/ml | 5 days |
| | Pyruvate | | |
| MOLM-13 | RPMI 1640 media 10% FBS, | 266 000 cells/ml | 3 days |
| | Glutamax and Sodium | | |
| | Pyruvate | | |
| MOLM-14 | RPMI 1640 media 10% FBS, | 250 000 cells/ml | 3 days |
| | Glutamax and Sodium | | |
| | Pyruvate | | |

TABLE 7
Compounds

| Compound | Supplier | Reference | Top dose | |
|---|---|---|---|---|
| Venetoclax (ABT-199) | Interchim | 1L2752-10MG | 10 | μM |
| Quizartinib | MedChemExpress | HY13001 | 0.1 | μM |
| Daunorubicin | Sigma-Aldrich | 30450-5MG | 10 | μM |
| Cytarabine | Bio-Techne | 4520/50mg | 10 | μM |
| 5-azacitidine | Sigma-Aldrich | A2385-100MG | 10 | μM |

### A. Cell Preparation

24 h before cell treatment, homogenize, stain with Trypan blue and count viable cells using CellOMeter Auto T4. Dilute cells in media at 400 000 cells/ml to ensure an exponential growth the day after.

On treatment day, homogenize, stain with Trypan blue and count viable cells using CellOMeter Auto T4. Dilute to the proper cell seeding density according to Table 6 in cell media.

### B. Cell Treatment

Dispense drug (see Table 7) and METTL3 inhibitor in semi-log dose-response matrix and DMSO in 384-well plates using D300e Nanodispenser (Tecan).
6× compound solution preparation: add 40 μL of cell medium in compound-containing plates and homogenize using the ViaFlo device (Integra)
Dispense 30 μl of cell suspension per well in three replicate 384-well white Ultra-Low-Attachment plates (Sbio) using ViaFlow device (Integra)
Transfer 6 μL of the 6× compound solution in each 30 μL cell suspension-containing plate.
Incubate at 37° C. 5% CO2 for 3 to 5 days.

### C. Cell Titer Glo Assay

Add 35 μL CellTiter-Glo (Promega) in wells (vol/vol) using ViaFlo device (Integra).
Incubate at RT 10 min in the dark, with agitation the first minute, and read luminescence on the Ensight device (Perkin Elmer).

### D. Data Analysis

Raw data were converted to % viability by normalization to DMSO controls. IC50 determination was performed using GraphPad Prism and fitting curves to the standard nonlinear regression four parameter equation (Y=Bottom+(Top-Bottom)/(1+(IC50/X)\hat{ }HillSlope)) least squares method, and synergy was assessed by comparison of IC50 values of the standard of care in the presence or absence of different concentrations of METTL3 inhibitor (see FIGS. 12 to 19).

### 1. Daunorubicin (Anthracycline Topoisomerase 2 Inhibitor)

Daunorubicin is a major component of the standard of care for AML treatment.

Additive interactions occur when the combination leads to an enhanced decrease in tumour cell viability with the addition of METTL3 inhibitor, yet the IC50 value does not change.

### Results

FIG. 12—STM3480 shows an additive interaction with daunorubicin in the Kasumi1 AML cell line, with increasing concentrations of STM3480 leading to decreasing viability. No change in IC50 of daunorubicin is observed, indicating an additive interaction.

In FIG. 12, the addition of STM3480 leads to a dose-dependent decrease in Kasumi1 cell viability. In the presence of 400 nM or 1265 nM STM3480, the daunorubicin IC50 did not significantly change, indicating an additive interaction.

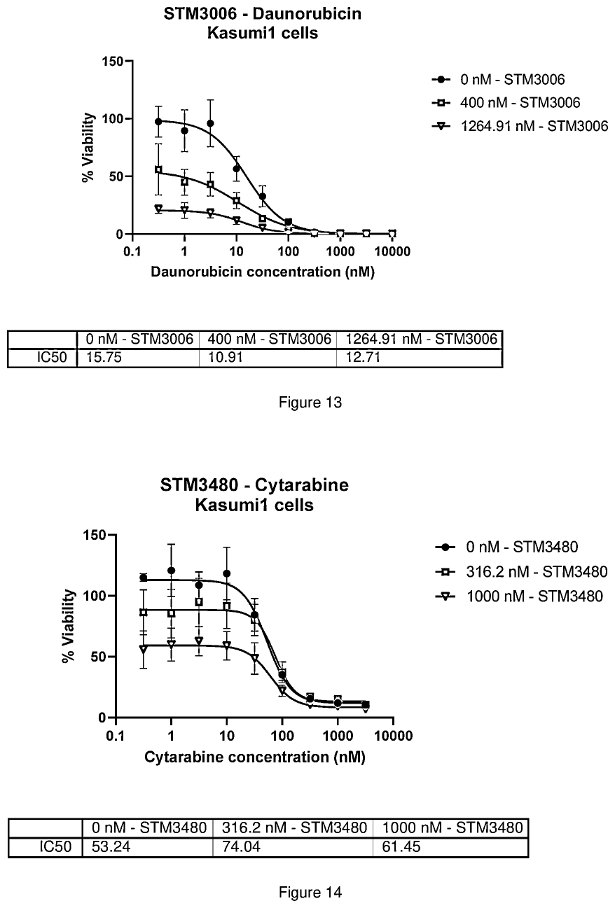

The same additive interaction was observed in the Kasumi1 cell line with additional METTL3 inhibitor STM3006 (see FIG. 13).

FIG. 13—STM3006 shows an additive interaction with daunorubicin in the Kasumi1 AML cell line, with increasing concentrations of STM3006 leading to decreasing viability. No change in IC50 of daunorubicin is observed, indicating an additive interaction.

### 2. Cytarabine (Nucleoside Analogue)

Cytarabine is a major component of the standard of care for AML treatment.

### Results

FIG. 14—STM3480 shows an additive interaction with cytarabine in the Kasumi1 AML cell line, with increasing concentrations of STM3480 leading to decreasing viability. No change in IC50 of cytarabine is observed, indicating an additive interaction.

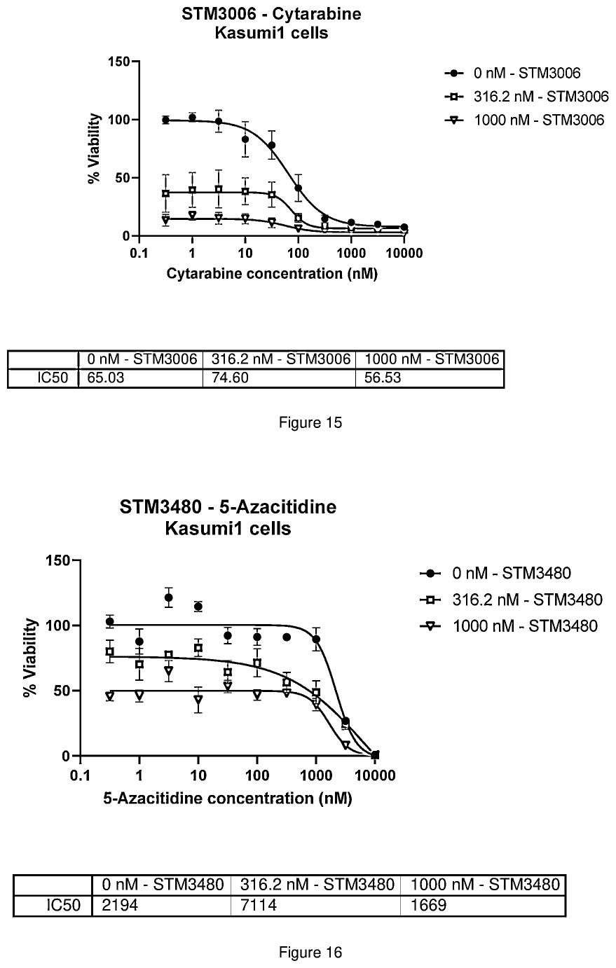

FIG. 15—STM3006 shows an additive interaction with cytarabine in the Kasumi1 AML cell line, with increasing concentrations of STM3006 leading to decreasing viability. No change in IC50 of cytarabine is observed, indicating an additive interaction.

### 3. 5′-Azacitidine (Hypomethylating Agent)

### Results

FIG. 16—STM3480 shows an additive interaction with 5′-azacitidine in the Kasumi1 AML cell line, with increasing concentrations of STM3480 leading to decreasing viability. No change in IC50 of 5′-azacitidine is observed, indicating an additive interaction.

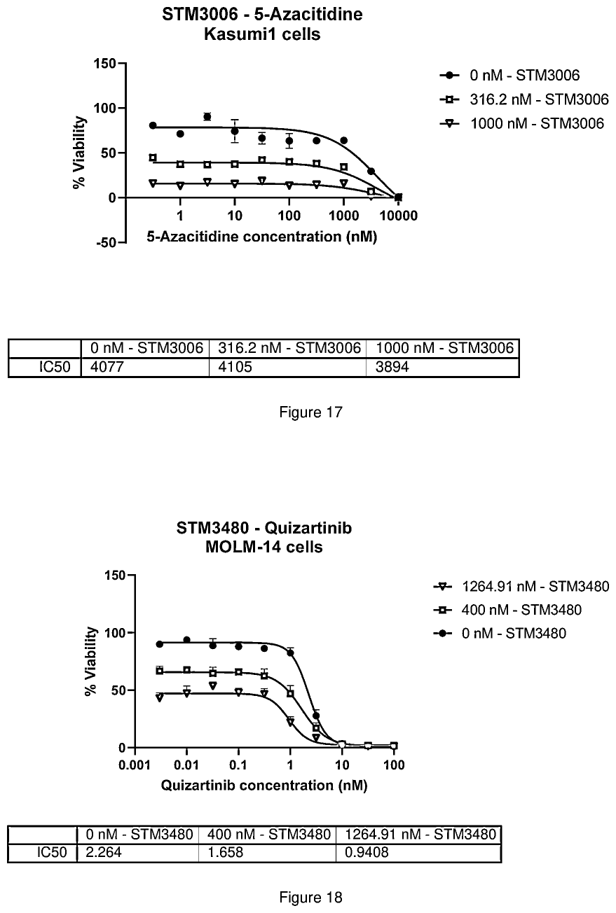

FIG. 17—STM3006 shows an additive interaction with 5′-azacitidine in the Kasumi1 AML cell line, with increasing concentrations of STM3006 leading to decreasing viability. No change in IC50 of 5′-azacitidine is observed, indicating an additive interaction.

### 4. Quizartinib (FLT3 Inhibitor)

MOLM-14 cells have a FLT3 mutation and are known to be highly dependent on FLT3 activity for survival and are highly sensitive to FLT3 inhibitors such as quizartinib.

### Results

FIG. 18—STM3480 shows an additive interaction with quizartinib in the FLT3-mutant MOLM-14 AML cell line, with increasing concentrations of STM3480 leading to decreasing viability. No change in IC50 of quizartinib is observed, indicating an additive interaction.

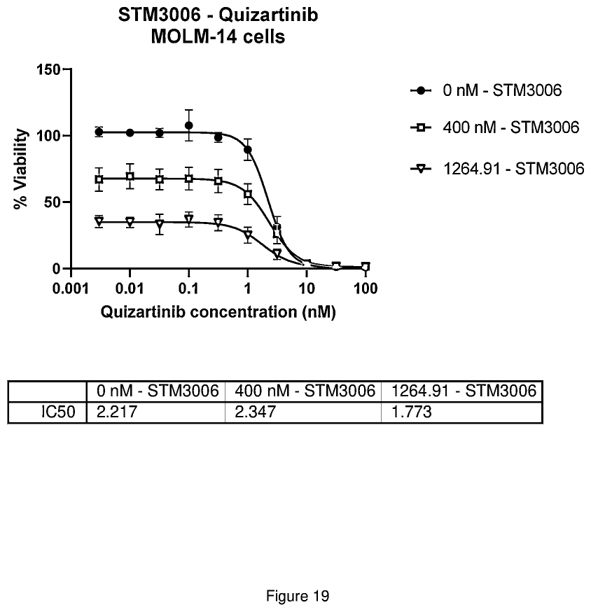

FIG. 19—STM3006 shows an additive interaction with quizartinib in the FLT3-mutant MOLM-14 AML cell line, with increasing concentrations of STM3006 leading to decreasing viability. No change in IC50 of quizartinib is observed, indicating an additive interaction.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1. A combination comprising a METTL3 inhibitor, or a pharmaceutically acceptable salt thereof, and:
(i) an immune oncology agent or therapy;
(ii) a BCL2 inhibitor, or a pharmaceutically acceptable salt thereof;
(iii) an anthracycline topoisomerase 2 inhibitor, or a pharmaceutically acceptable salt thereof;
(iv) cytarabine, or a pharmaceutically acceptable salt thereof;
(v) a hypomethylating agent, or a pharmaceutically acceptable salt thereof; or
(vi) a FLT3 inhibitor, or a pharmaceutically acceptable salt thereof.

2. A combination according to claim 1, wherein the immune oncology agent or therapy is selected from the group consisting of immune checkpoint inhibitors (e.g. a PD1, PD-L1 inhibitor, LAG3, CTLA-4, TIGIT, TIM3, or VISTA inhibitor), STING agonists, TLR agonists, anti-CD137 antibodies, CD28 antibodies, OX40 stimulators, CD40 antibodies, ICOS agonists, GITR agonists, A₂AR antagonists, Bispecific T cell engagers (BiTE), oncolytic viruses, cancer vaccines, and/or CAR-T cell therapy.

3. A combination according to claim 1 or claim 2, wherein the immune oncology agent or therapy is treatment with an immune checkpoint inhibitor, or a pharmaceutically acceptable salt thereof.

4. A combination according to claim 2 or claim 3, wherein the immune checkpoint inhibitor is selected from a PD1, PD-L1 inhibitor, a LAG3 inhibitor and a CTLA-4 inhibitor.

5. A combination according to any one of claims 2 to 4, wherein the immune checkpoint inhibitor is selected from a PD1 or PD-L1 inhibitor.

6. A combination according to any one of claims 2 to 4, wherein the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab and durvalumab, or a pharmaceutically acceptable salt thereof.

7. A combination according to claim 6, wherein the immune checkpoint inhibitor is selected from pembrolizumab, nivolumab, atezolizumab, avelumab and durvalumab, or a pharmaceutically acceptable salt thereof.

8. A combination according to claim 1, wherein the BCL2 inhibitor is venetoclax, or a pharmaceutically acceptable salt thereof.

9. A combination according to claim 1, wherein:
the anthracycline topoisomerase 2 inhibitor is selected from daunorubicin, doxorubicin, epirubicin or idarubicin, or a pharmaceutically acceptable salt thereof;
the hypomethylating agent is selected from 5-azacitidine or decitabine, or a pharmaceutically acceptable salt thereof;
the FLT3 inhibitor is selected from sorafenib, lestaurtinib, midostaurin, quizartinib, crenolanib, or gilteritinib, or a pharmaceutically acceptable salt thereof.

10. A combination according to any one of claims 1 to 9, wherein the METTL3 inhibitor is a compound of formula I, II, VI or VII defined herein, or a pharmaceutically acceptable salt thereof.

11. A combination according to any one of claims 1 to 9, wherein the METTL3 inhibitor is selected from STM3480, STM3675 or STM3006, or a pharmaceutically acceptable salt thereof.

12. A pharmaceutical product comprising a combination according to any one of claims 1 to 11.

13. A pharmaceutical composition comprising a combination according to any one of claims 1 to 11, and one or more pharmaceutically acceptable excipients.

14. A METTL3 inhibitor, or a pharmaceutically acceptable salt thereof, for use in the treatment of cancer, wherein the METTL3 inhibitor, or a pharmaceutically acceptable salt thereof, is administered in combination with:
(i) an immune oncology agent or therapy as claimed in any one of claims 1 to 7;
(ii) a BCL2 inhibitor, or a pharmaceutically acceptable salt thereof, as claimed in claim 1 or claim 8;
(iii) an anthracycline topoisomerase 2 inhibitor, or a pharmaceutically acceptable salt thereof as claimed in claim 1 or claim 9;
(iv) cytarabine, or a pharmaceutically acceptable salt thereof as claimed in claim 9;
(v) a hypomethylating agent, or a pharmaceutically acceptable salt thereof as claimed in claim 1 or claim 9; or
(vi) a FLT3 inhibitor, or a pharmaceutically acceptable salt thereof as claimed in claim 1 or claim 9.

15. A METTL3 inhibitor, or a pharmaceutically acceptable salt thereof, for use in the treatment of cancer according to claim 14, wherein the METTL3 inhibitor, or a pharmaceutically acceptable salt thereof, is administered in combination with an immune checkpoint inhibitor.

16. A METTL3 inhibitor, or a pharmaceutically acceptable salt thereof, for use in the treatment of a solid tumour, wherein the METTL3 inhibitor, or a pharmaceutically acceptable salt thereof, is administered in combination with an immune checkpoint inhibitor.

17. A METTL3 inhibitor, or a pharmaceutically acceptable salt thereof, for use in the treatment of cancer according to claim 15 or a solid tumour according to claim 16, wherein the METTL3 inhibitor is selected from STM3480, STM3675 or STM3006, or a pharmaceutically acceptable salt thereof, and is administered in combination with an immune checkpoint inhibitor, or a pharmaceutically acceptable salt thereof, as defined in any one of claims 4 to 7.

18. A METTL3 inhibitor, or a pharmaceutically acceptable salt thereof, for use in the treatment of cancer according to claim 14, wherein the METTL3 inhibitor, or a pharmaceutically acceptable salt thereof, is administered in combination with a BCL2 inhibitor, or a pharmaceutically acceptable salt thereof, as claimed in claim 1 or claim 8.

19. A METTL3 inhibitor, or a pharmaceutically acceptable salt thereof, for use in the treatment of cancer according to claim 18, wherein the METTL3 inhibitor is selected from STM3480, STM3675 or STM3006, or a pharmaceutically acceptable salt thereof, and is administered in combination with venetoclax, or a pharmaceutically acceptable salt thereof.

20. STM3480, or a pharmaceutically acceptable salt thereof, for use in the treatment of acute myeloid leukaemia (AML), chronic lymphocytic leukaemia (CLL), small lymphocytic lymphoma (SLL) or myelodysplastic syndromes (MDS), wherein STM3480 is administered in combination with venetoclax, or a pharmaceutically acceptable salt thereof.

21. STM3480, or a pharmaceutically acceptable salt thereof, for use in the treatment of acute myeloid leukaemia (AML), wherein STM3480 is administered in combination with venetoclax, or a pharmaceutically acceptable salt thereof.

22. A METTL3 inhibitor, or a pharmaceutically acceptable salt thereof, for use in the treatment of cancer according to claim 14, wherein the METTL3 inhibitor, or a pharmaceutically acceptable salt thereof, is administered in combination with
(i) an anthracycline topoisomerase 2 inhibitor, or a pharmaceutically acceptable salt thereof as claimed in claim 1 or claim 9;
(ii) cytarabine, or a pharmaceutically acceptable salt thereof, as claimed in claim 9;
(iii) a hypomethylating agent, or a pharmaceutically acceptable salt thereof, as claimed in claim 1 or claim 9; or
(iv) a FLT3 inhibitor, or a pharmaceutically acceptable salt thereof, as claimed in claim 1 or claim 9.

23. A METTL3 inhibitor, or a pharmaceutically acceptable salt thereof, for use in the treatment of cancer according to claim 22, wherein the METTL3 inhibitor is STM3480.

24. A METTL3 inhibitor, or a pharmaceutically acceptable salt thereof, for use as an immune-sensitiser.
